(19) United States
(12) Patent Application Publication
(10) Pub. No.: US 2025/0250257 A1
(43) Pub. Date: Aug. 7, 2025

(54) NOVEL CRYSTALLINE FORMS

(71) Applicant: Assembly Biosciences, Inc., South San Francisco, CA (US)

(72) Inventors: Zhixin ZONG, South San Francisco, CA (US); Yi WU, South San Francisco, CA (US); Noorullah Naqvi MOHAMMED, South San Francisco, CA (US); Nicole WHITE, South San Francisco, CA (US); Mark JOHNSON, South San Francisco, CA (US); Michael WALLACE, South San Francisco, CA (US)

(73) Assignee: Assembly Biosciences, Inc., South San Francisco, CA (US)

(21) Appl. No.: 18/702,908

(22) PCT Filed: Oct. 19, 2022
(86) PCT No.: PCT/IB2022/060055

§ 371 Date: Apr. 19, 2024

Related U.S. Application Data
(60) Provisional application No. 63/257,723, filed on Oct. 20, 2021.

(51) Int. Cl. C07D 403/08

(52) U.S. Cl. CPC C07D 403/08

(57) ABSTRACT

The present disclosure generally relates to novel crystalline forms of a certain compound which are useful as modulators of hepatitis B virus core protein assembly. The present disclosure also relates to identifying suitable solid forms with promising solid state properties for clinical development. The solid state forms disclosed may be used in the manufacture of drug products which may have allosteric effector properties against hepatitis B virus (HBV) core protein (Cp), a protein found as a dimer, a multimer, and as the protein shell of the HBV core. As one example, provided herein is a stable crystalline form which may be useful for treating viral infections, such as hepatitis B.

### FIELD OF THE INVENTION

The present disclosure generally relates to novel crystalline forms of a certain compound, which are useful as modulators of hepatitis B virus core protein assembly. The present disclosure also relates to identifying suitable solid forms with promising solid state properties for clinical development. The solid state forms disclosed may be used in the manufacture of drug products which may have allosteric effector properties against hepatitis B virus (HBV) core protein (Cp), a protein found as a dimer, a multimer, and as the protein shell of the HBV core. As one example, provided herein is a stable crystalline form which may be useful for treating viral infections, such as hepatitis B.

### BACKGROUND

Hepatitis B (HBV) causes viral hepatitis that can further lead to chronic liver disease and increase the risk of liver cirrhosis and liver cancer (hepatocellular carcinoma). Worldwide, about 2 billion people have been infected with HBV, around 360 million people are chronically infected, and every year HBV infection causes more than one half million deaths. HBV can be spread by body fluids: from mother to child, by sex, and via blood products. Children born to HBV-positive mothers may also be infected, unless vaccinated at birth.

The hepatitis virus particle is composed of a lipid envelope studded with surface protein (HBsAg) that surrounds the viral core. The core is composed of a protein shell, or capsid, built of 120 core protein (Cp) dimers, which in turn contains the relaxed circular DNA (rcDNA) viral genome as well as viral and host proteins. In an infected cell, the genome is found as a covalently closed circular DNA (cccDNA) in the host cell nucleus. The cccDNA is the template for viral RNAs and thus viral proteins. In the cytoplasm, Cp assembles around a complex of full-length viral RNA (the so-called pregenomic RNA or pgRNA and viral polymerase (P). After assembly, P reverse transcribes the pgRNA to rcDNA within the confines of the capsid to generate the DNA-filled viral core.

At present, chronic HBV is primarily treated with nucleos(t)ide analogs (e.g., entecavir) that suppress the virus while the patient remains on treatment, but do not eliminate the infection, even after many years of treatment. Once a patient starts taking nucleos(t)ide analogs, most must continue taking them or risk the possibility of a life-threatening immune response due to viral rebound. Further, nucleotide therapy may lead to the emergence of antiviral drug resistance.

The only FDA approved alternative to nucleos(t)ide analogs is treatment with interferon α or pegylated interferon α. Unfortunately, the adverse event incidence and profile of interferon α can result in poor tolerability, and many patients are unable to complete therapy. Moreover, only a small percentage of patients are considered appropriate for interferon therapy, as only a small subset of patients is likely to have a sustained clinical response to a course of interferon therapy. As a result, interferon-based therapies are used in only a small percentage of all diagnosed patients who elect treatment.

Thus, current HBV treatments can range from palliative to watchful waiting. Nucleotide analogs suppress virus production, treating the symptom, but leave the infection intact. Interferon α has severe side effects and less tolerability among patients and is successful as a finite treatment strategy in only a small minority of patients. There is a clear on-going need for more effective treatments for HBV infections.

The present disclosure relates to novel crystalline forms of a certain compound. The compound was first disclosed in PCT/US2021/028323, which is hereby incorporated by reference in its entirety.

### SUMMARY OF THE INVENTION

One embodiment of the present disclosure is a crystalline form of Compound I

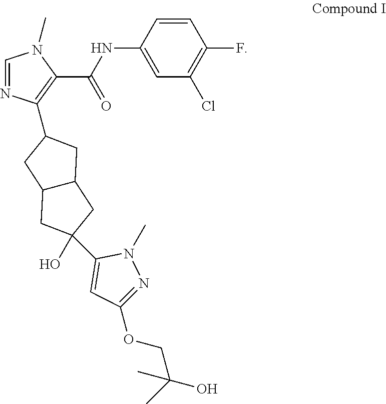 [SMILES: Cn1cnc(C2CC3CC(O)(c4cc(OCC(C)(C)O)nn4C)CC3C2)c1C(=O)Nc1ccc(F)c(Cl)c1]

One embodiment of the present disclosure is a crystalline form of Compound I, wherein the crystalline form is a crystalline form of Compound I(a):

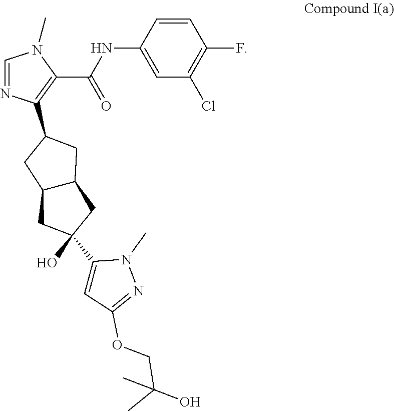 [SMILES: Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

The present disclosure relates to certain novel crystalline solid forms of Compound I, suitably Compound I(a), which possess promising and advantageous solid-state and/or biopharmaceutical properties.

The present disclosure is also directed to pharmaceutical compositions comprising a crystalline form or a mixture of crystalline forms, and to methods for preparing such forms. The present disclosure is further directed to the use of the crystalline forms in the treatment of HBV infections.

It is understood that there are a number of analytical methods one of ordinary skill in the art in solid-state chemistry can use to analyze solid forms. The term “analyze” as used herein means to obtain information about the solid-state structure of solid forms. For example, powder X-ray diffraction (PXRD) is a suitable technique for differentiating amorphous solid forms from crystalline solid forms and for characterizing and identifying particular crystalline solid forms of a compound.

Due to differences in instruments, samples, and sample preparation, PXRD peak values are often reported with the modifier “±0.2° 2θ”. This is common practice in the solid-state chemical arts because of the variation inherent in peak values. Variability in peak intensity is a result of how individual crystals are oriented in the sample container with respect to the external X-ray source (known as “preferred orientation”). This orientation effect does not provide structural information about the crystal.

It should be noted that, unless otherwise stated, X-Ray Powder Diffractograms disclosed herein were acquired using Cu Kα (1.5406 Å) radiation.

It is also understood that PXRD is just one of several analytical techniques one may use to characterize and/or identify crystalline solid forms. For example, Differential Scanning calorimetry (DSC) may also be used to characterize and/or identity crystalline solid forms. A typical variability for a value associated with a differential scanning calorimetry onset temperature is in the order of plus or minus 2° C.

It should be noted that unless noted otherwise, thermal data (DSC and TGA) presented herein were acquired using a heating rate of 10° C./min. Furthermore, DSC data was acquired using aluminum crimped pans, while TGA data was acquired using platinum, open pans.

In one aspect of the present invention, the crystalline form is Type 1. Type 1 is an anhydrous crystalline form. In one embodiment, Type 1 is an anhydrous crystalline form of Compound I, wherein Compound I is Compound I(a).

In one aspect, the crystalline form Type 1 is characterized by the XRPD pattern of FIG. 24. In one aspect, the crystalline form Type 1 is characterized by an XRPD having at least one of the 2θ peaks from Table 1:

TABLE 1
XRPD peak positions for Type 1.

| peak | 2Theta (degree) |
|---|---|
| 1 | 7.34 |
| 2 | 12.17 |
| 3 | 16.43 |
| 4 | 17.4 |
| 5 | 17.65 |
| 6 | 18.11 |
| 7 | 19.37 |
| 8 | 19.87 |

In one aspect, the crystalline form Type 1 is characterized by at least two of the 2θ peaks from Table 1. In one aspect, the crystalline form Type 1 is characterized by at least three of the 2θ peaks from Table 1. In one aspect, the crystalline form Type 1 is characterized by at least four of the 2θ peaks from Table 1. In one aspect, the crystalline form Type 1 is characterized by at least five of the 2θ peaks from Table 1. In one aspect, the crystalline form Type 1 is characterized by at least six of the 2θ peaks from Table 1. In one aspect, the crystalline form Type 1 is characterized by at least seven of the 2θ peaks from Table 1. In one aspect, the crystalline form Type 1 is characterized by the eight 2θ peaks from Table 1.

In one aspect, the crystalline form Type 1 is characterized by an XRPD pattern measured using Cu Kα (1.5406 Å) radiation comprising peaks at 2-theta values of 12.2, 19.4 and 19.9° 2θ±0.2° 2θ (suitably °2θ±0.1°2θ). In a further aspect, the crystalline form Type 1 is characterized by an XRPD pattern measured using Cu Kα (1.5406 Å) radiation comprising peaks at 2-theta values of 12.2, 19.4 and 19.9° 2θ±0.2° 2θ (suitably °2θ±0.1°2θ) and further comprising at least one or two specific peaks selected from the peaks at 2-theta values of 16.4 and 17.6° 2θ±0.2° 2θ (suitably ° 2θ±0.1° 2θ).

In one aspect, the crystalline form Type 1 is characterized by an XRPD pattern measured using Cu Kα (1.5406 Å) radiation comprising peaks at 2-theta values of 12.2, 19.4 and 19.9° 2θ±0.2° 2θ (suitably ° 2θ±0.1°2θ) and further comprises at least two, five, ten, fifteen or twenty additional peaks selected from the group consisting of the peaks in Table 2 in ° 2θ±0.2° 2θ (suitably ° 2θ±0.1° 2θ).

TABLE 2
XRPD peak positions for Type 1

| Peak | Peak |
|---|---|
| Position | Position |
| in °2θ | in °2θ |
| 4.2 | 24.1 |
| 5.0 | 24.3 |
| 5.8 | 25.0 |
| 6.1 | 26.2 |
| 7.3 | 26.5 |
| 10.7 | 26.8 |
| 11.2 | 27.5 |
| 12.9 | 28.1 |
| 13.5 | 28.5 |
| 14.0 | 29.5 |
| 14.9 | 30.2 |
| 15.4 | 31.0 |
| 16.4 | 32.2 |
| 17.0 | 33.1 |
| 17.4 | 33.5 |
| 17.7 | 34.2 |
| 18.1 | 34.4 |
| 20.7 | 34.9 |
| 20.9 | 35.6 |
| 21.4 | 36.2 |
| 21.7 | 36.9 |
| 21.9 | 37.3 |
| 22.9 | 37.8 |
| 23.4 | |

In one aspect, the crystalline form Type 1 is anhydrous and is characterized by an XRPD pattern measured using Cu Kα (1.5406 Å) radiation comprising peaks at 2-theta values of 12.2, 19.4 and 19.9° 2θ±0.2° 2θ (suitably ° 2θ±0.1°2θ). In one aspect, the crystalline form Type 1 is characterized by an XRPD pattern measured using Cu Kα (1.5406 Å) radiation comprising peaks at 2-theta values of 12.2, 19.4 and 19.9° 2θ±0.2° 2θ (suitably ° 2θ±0.1° 2θ), and wherein Type 1 comprises less than 2% by weight of water. In one aspect, the crystalline form Type 1 is characterized by an XRPD pattern measured using Cu Kα (1.5406 Å) radiation comprising peaks at 2-theta values of 12.2, 19.4 and 19.9° 2θ±0.2° 2θ (suitably ° 2θ±0.1° 2θ), and wherein Type 1 comprises less than 1.5% by weight of water. In a further aspect, the crystalline form Type 1 is characterized by an XRPD pattern measured using Cu Kα (1.5406 Å) radiation comprising peaks at 2-theta values of 12.2, 19.4 and 19.9° 2θ±0.2° 2θ (suitably ° 2θ±0.1°2θ) and further comprising at least one or two specific peaks selected from the peaks at 2-theta values of 16.4 and 17.6° 2θ±0.2° 2θ (suitably ° 2θ±0.1°2θ), and wherein Type 1 comprises less than 2% by weight of water. In a further aspect, the crystalline form Type 1 is characterized by an XRPD pattern measured using Cu Kα (1.5406 Å) radiation comprising peaks at 2-theta values of 12.2, 19.4 and 19.9° 2θ±0.2° 2θ (suitably ° 2θ±0.1°2θ) and further comprising at least one or two specific peaks selected from the peaks at 2-theta values of 16.4 and 17.6° 2θ±0.2° 2θ (suitably ° 2θ±0.1° 2θ), and wherein Type 1 comprises less than 1.5% by weight of water.

In one aspect, the crystalline form Type 1 is characterized by an XRPD pattern measured using Cu Kα (1.5406 Å) radiation substantially the same as shown in FIG. 24.

In one aspect, the crystalline form Type 1 comprises less than 2% by weight of water, such as less than 1.5% or less than 1.0%. The skilled person would know of suitable analytical techniques which can quantify the amount of water associated with a solid. For example, water content can be determined by Karl Fischer Titration. Thermogravimetric Analysis (TGA) can also quantify the amount of volatile material (i.e., water) associated with a solid (either surface bound or incorporated into the crystal structure).

In one aspect, the crystalline form Type 1 is characterized by a DSC thermogram comprising an endothermic event with an onset temperature of 210° C.±2° C. (suitably ±1° C.). In one aspect, the crystalline form Type 1 is characterized by a DSC thermogram comprising no thermal events between 5° and 100° C. and an endothermic event with an onset temperature of 210° C.±2° C. (suitably ±1° C.).

In one aspect, the crystalline form Type 1 is characterized by a DSC thermogram substantially the same as the DSC thermogram depicted in FIG. 2B.

In one aspect, the crystalline form Type 1 is at least 75%, 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% free of other forms. In one aspect, the crystalline form Type 1 is free from other forms.

In one aspect of the present invention, the crystalline form is Type 2. Type 2 is a hydrated crystalline form. In one embodiment, Type 2 is a crystalline tri-hydrate form of Compound I, wherein Compound I is Compound I(a). In one aspect, the crystalline form Type 2 is characterized by the XRPD pattern of FIG. 25. In one aspect, the crystalline form Type 2 is characterized by an XRPD having at least one of the 2θ peaks from Table 3:

TABLE 3
XRPD peak positions for Type 2

| peak | 2Theta (degree) |
|---|---|
| 1 | 5.59 |
| 2 | 6.16 |
| 3 | 8.53 |
| 4 | 14.15 |
| 5 | 16.14 |

In one aspect, the crystalline form Type 2 is characterized by at least two of the 2θ peaks from Table 3. In one aspect, the crystalline form Type 2 is characterized by at least three of the 20 peaks from Table 3. In one aspect, the crystalline form Type 2 is characterized by at least four of the 2θ peaks from Table 3. In one aspect, the crystalline form Type 2 is characterized by the five 2θ peaks from Table 3.

In one aspect, the crystalline form Type 2 is characterized by an XRPD pattern measured using Cu Kα (1.5406 Å) radiation comprising peaks at 2-theta values of 5.6 and 8.5° 2θ±0.2° 2θ(suitably ° 2θ±0.1° 2θ). In one aspect, the crystalline form Type 2 is characterized by an XRPD pattern measured using Cu Kα (1.5406 Å) radiation comprising peaks at 2-theta values of 5.6 and 8.5° 2θ±0.2° 2θ (suitably °2θ±0.1°2θ) and further comprises at least two, five, ten, fifteen or twenty additional peaks selected from the group consisting of the peaks in Table 4 in ° 2θ±0.2° 2θ(suitably ° 2θ±0.1° 2θ).

TABLE 4
XRPD peak positions for Type 2
Peak Position in °2θ

4.3
6.2
8.1
9.9
11.1
12.3
13.2
14.2
14.9
16.1
16.9
17.7
18.2
18.6
19.6
20.3
21.0
21.4
21.6
22.1
23.2
23.5
24.3
24.7
25.5
26.2
26.8
27.2
28.1
29.1
30.0
31.2
33.5
34.7
38.3

In one aspect, the crystalline form Type 2 is characterized by an XRPD pattern measured using Cu Kα (1.5406 Å) radiation substantially the same as shown in FIG. 25.

In one aspect, the crystalline form Type 2 comprises between 5 and 12% by weight of water, such as between 7 and 11% by weight. In one aspect, the crystalline form Type 2 comprises about 9% by weight of water.

In one aspect, the crystalline form Type 2 is characterized by a DSC thermogram comprising an endothermic event with an onset temperature of 212° C.±2° C. (suitably ±1° C.).

In one aspect, the crystalline form Type 2 is characterized by a DSC thermogram comprising an endothermic event between 5° and 120° C. and an endothermic event with an onset temperature of 212° C.±2° C. (suitably ±1° C.).

In one aspect, the crystalline form Type 2 is characterized by a DSC thermogram substantially the same as the DSC thermogram depicted in FIG. 4B.

In one aspect, the crystalline form Type 2 is at least 75%, 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% free of other forms. In one aspect, the crystalline form Type 2 is free from other forms.

In one aspect of the present invention, the crystalline form is Type 3. Type 3 is a hydrated crystalline form. In one embodiment, Type 3 is a crystalline mono-hydrate of Compound I. In one embodiment, Type 3 is a crystalline mono-hydrate of Compound I, wherein Compound I is Compound I(a).

In one aspect, the crystalline form Type 3 is characterized by the XRPD pattern of FIG. 26. In one aspect, the crystalline form Type 3 is characterized by an XRPD having at least one of the 2θ peaks from Table 5:

TABLE 5
XRPD peak positions for Type 3

| peak | 2Theta (degree) |
|---|---|
| 1 | 6.38 |
| 2 | 7.12 |
| 3 | 14.83 |

In one aspect, the crystalline form Type 3 is characterized by at least two of the 2θ peaks from Table 5. In one aspect, the crystalline form Type 3 is characterized by the three 2θ peaks from Table 5.

In one aspect, the crystalline form Type 3 is characterized by an XRPD pattern measured using Cu Kα (1.5406 Å) radiation comprising peaks at 2-theta values of 6.4 and 7.1 °2θ±0.2° 2θ(suitably °2θ±0.1° 2θ In one aspect, the crystalline form Type 3 is characterized by an XRPD pattern measured using Cu Kα (1.5406 Å) radiation comprising peaks at 2-theta values of 6.4 and 7.1° 2θ±0.2° 2θ (suitably °2θ±0.1°2θ) and further comprises at least one or two specific peaks selected from the peaks at 2-theta values of 9.7, 14.2 and 14.9° 2θ±0.2° 2θ (suitably °2θ±0.1°2θ).

In one aspect, the crystalline form Type 3 is characterized by an XRPD pattern measured using Cu Kα (1.5406 Å) radiation comprising peaks at 2-theta values of 6.4 and 7.1 °2θ±0.2° 2θ(suitably ° 2θ±0.1° 2θ) and at least two, five, ten or fifteen additional peaks selected from the group consisting of the peaks in Table 6 in ° 2θ±0.2° 2θ (suitably ° 2θ±0.1°2θ).

TABLE 6
XRPD peak positions for Type 3
Peak Position in °2θ

8.5
9.3
9.7
10.9
13.4
14.2
14.9
15.8
16.3
18.3
20.4
20.9
21.4
22.0
22.9
25.8
28.7
29.9
31.6
33.7

In one aspect, the crystalline form Type 3 is characterized by an XRPD pattern measured using Cu Kα (1.5406 Å) radiation substantially the same as shown in FIG. 26.

In one aspect, the crystalline form Type 3 comprises between 2 and 4% by weight of water. In one aspect, the crystalline form Type 3 comprises about 3% by weight of water.

In one aspect, the crystalline form Type 3 is characterized by a DSC thermogram comprising endothermic events with onset temperatures of 192 and 212° C.±2° C. (suitably ±1° C.).

In one aspect, the crystalline form Type 3 is characterized by a DSC thermogram substantially the same as the DSC thermogram depicted in FIG. 7B.

In one aspect, the crystalline form Type 3 is at least 75%, 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% free of other forms. In one aspect, the crystalline form Type 3 is free from other forms.

In one aspect of the present invention, the crystalline form is Type 4. Type 4 is a hydrated crystalline form. In one embodiment, Type 4 is a crystalline sesqui-hydrate of Compound I. In one embodiment, Type 4 is a crystalline sesqui-hydrate of Compound I, wherein Compound I is Compound I(a).

In one aspect, the crystalline form Type 4 is characterized by the XRPD pattern of FIG. 27. In one aspect, the crystalline form Type 4 is characterized by an XRPD having at least one of the 2θ peaks from Table 7:

TABLE 7
XRPD peak positions for Type 4

| peak | 2Theta (degree) |
|---|---|
| 1 | 5.03 |
| 2 | 8.24 |
| 3 | 10.16 |
| 4 | 13.88 |
| 5 | 14.46 |

In one aspect, the crystalline form Type 4 is characterized by at least two of the 2θ peaks from Table 7. In one aspect, the crystalline form Type 4 is characterized by at least three of the 20 peaks from Table 7. In one aspect, the crystalline form Type 4 is characterized by at least four of the 2θ peaks from Table 7. In one aspect, the crystalline form Type 4 is characterized by the five 2θ peaks from Table 7.

In one aspect, the crystalline form Type 4 is characterized by an XRPD pattern measured using Cu Kα (1.5406 Å) radiation comprising peaks at 2-theta values of 5.1 and 17.6° 2θ±0.2° 2θ(suitably ° 2θ±0.1°2θ). In one aspect, the crystalline form Type 4 is characterized by an XRPD pattern measured using Cu Kα (1.5406 Å) radiation comprising peaks at 2-theta values of 5.1 and 17.6° 2θ±0.2° 2θ (suitably ° 2θ±0.1°2θ) and further comprising a peak at 2-theta value of 8.3° 2θ±0.2° 2θ (suitably ° 2θ±0.1°2θ).

In one aspect, the crystalline form Type 4 is characterized by an XRPD pattern measured using Cu Kα (1.5406 Å) radiation comprising peaks at 2-theta values of 5.1 and 17.6° 2θ±0.2° 2θ(suitably ° 2θ±0.1°2θ) and at least two, five, ten, fifteen or twenty additional peaks selected from the group consisting of the peaks in Table 8 in ° 2θ±0.2° 2θ (suitably °2θ±0.1°2θ).

TABLE 8
XRPD peak positions for Type 4
Peak Position in °2θ

6.2
7.3
8.3
9.4
10.0
11.5
12.2
13.0
13.9
14.5
15.0
15.4
16.4
17.3
18.1
18.7
19.3
19.9
20.7
21.9
22.5
23.4
24.3
25.7
26.5
28.5
29.5
30.6
31.0
32.2
34.3
39.6

In one aspect, the crystalline form Type 4 is characterized by an XRPD pattern measured using Cu Kα (1.5406 Å) radiation substantially the same as shown in FIG. 27.

In one aspect, the crystalline form Type 4 comprises between 4 and 6% by weight of water. In one aspect, the crystalline form Type 4 comprises about 4.5% by weight of water.

In one aspect, the crystalline form Type 4 is characterized by a DSC thermogram comprising an endothermic event with onset temperature of 210° C.±2° C. (suitably ±1° C.). In one aspect, the crystalline form Type 4 is characterized by a DSC thermogram comprising an endothermic event between 75 and 110° C.±2° C. (suitably ±1° C.) and an endothermic event with onset temperature of 210° C.±2° C. (suitably ±1° C.).

In one aspect, the crystalline form Type 4 is characterized by a DSC thermogram substantially the same as the DSC thermogram depicted in FIG. 8B.

In one aspect, the crystalline form Type 4 is at least 75%, 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% free of other forms. In one aspect, the crystalline form Type 4 is free from other forms.

In one aspect of the present invention, the crystalline form is Type 5. Type 5 is a solvated crystalline form. In one embodiment, Type 5 is a crystalline mono-dichloromethane solvate of Compound I. In one embodiment, Type 5 is a crystalline mono-dichloromethane solvate of Compound I, wherein Compound I is Compound of I(a).

In one aspect, the crystalline form Type 5 is characterized by the XRPD pattern of FIG. 28. In one aspect, the crystalline form Type 5 is characterized by an XRPD having at least one of the 2θ peaks from Table 9:

TABLE 9
XRPD peak positions for Type 5

| peak | 2Theta (degree) |
|---|---|
| 1 | 6.6 |
| 2 | 18.9 |

In one aspect, the crystalline form Type 5 is characterized by the two 2θ peaks from Table 9.

In one aspect, the crystalline form Type 5 is characterized by an XRPD pattern measured using Cu Kα (1.5406 Å) radiation comprising a peak at 2-theta value of 6.7° 2θ±0.2° 2θ (suitably ° 2θ±0.1°2θ). In one aspect, the crystalline form Type 5 is characterized by an XRPD pattern measured using Cu Kα (1.5406 Å) radiation comprising peaks at 2-theta values of 6.7 and 18.9° 2θ±0.2° 2θ (suitably ° 2θ±0.1° 2θ).

In one aspect, the crystalline form Type 5 is characterized by an XRPD pattern measured using Cu Kα (1.5406 Å) radiation comprising a peak at 2-theta value of 6.7 9° 2θ±0.2° 2θ (suitably ° 2θ±0.1° 2θ) and at least two, five, ten, fifteen or twenty additional peaks selected from the group consisting of the peaks in Table 10 in ° 2θ±0.2° 2θ (suitably ° 2θ±0.1°2θ).

TABLE 10
XRPD peak positions for Type 5
Peak Position in °2θ

5.1
5.7
8.0
9.1
9.8
10.1
10.7
11.9
12.6
14.0
14.9
15.6
16.5
17.3
18.9
20.6
21.4
23.3
24.3
26.3
27.6
27.9
28.7
30.2

In one aspect, the crystalline form Type 5 is characterized by an XRPD pattern measured using Cu Kα (1.5406 Å) radiation substantially the same as shown in FIG. 28.

In one aspect, the crystalline form Type 5 comprises between 11 and 15% by weight of dichloromethane. In one aspect, the crystalline form Type 5 comprises about 13% by weight of dichloromethane.

In one aspect, the crystalline form Type 5 is characterized by a DSC thermogram comprising an endothermic event with an onset temperature of 210° C.±2° C. (suitably ±1° C.). In one aspect, the crystalline form Type 5 is characterized by a DSC thermogram comprising an endothermic event between 7° and 110° C.±2° C. (suitably ±1° C.), an exothermic event between 16° and 200° C.±2° C. (suitably ±1° C.), and an endothermic event with an onset temperature of 210° C.±2° C. (suitably ±1° C.).

In one aspect, the crystalline form Type 5 is characterized by a DSC thermogram substantially the same as the DSC thermogram depicted in FIG. 9B.

In one aspect, the crystalline form Type 5 is at least 75%, 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% free of other forms. In one aspect, the crystalline form Type 5 is free from other forms.

In one aspect of the present invention, the crystalline form is Type 8. Type 8 is an anhydrous crystalline form. In one embodiment, Type 8 is an anhydrous crystalline form of Compound I, wherein the Compound I is Compound I(a).

Type 8 was surprisingly produced during a drug substance manufacturing campaign. A manufacturing batch, with the aim to produce a different form, returned trace amounts of another crystalline form, which was later characterized as a new solid state form, i.e. Type 8. Type 8 had not previously been observed during the numerous crystallization experiments described in Example 2.

The applicants characterized Type 8 and found that it is the most thermodynamically stable form discovered to date.

In one aspect, the crystalline form Type 8 is characterized by the XRPD pattern measured using Cu Kα (2=1.5406 Å) radiation of FIG. 29. In one aspect, the crystalline form Type 8 is characterized by an XRPD pattern measured using Cu Kα (λ=1.5406 Å) radiation comprising peaks at 2-theta values of 9.4, 15.4 and 16.7° 2θ±0.2° 2θ (suitably ±0.1°20).

In one aspect, the crystalline form Type 8 is characterized by an XRPD pattern measured using Cu Kα (2=1.5406 Å) radiation comprising peaks at 2-theta values of 9.4, 15.4 and 16.7 °2θ±0.2° 2θ (suitably ±0.1°20) and at least two, five, ten, fifteen or twenty additional peaks selected from the group consisting of the peaks in Table 11 in ° 2θ±0.2° 2θ (suitably °2θ±0.1°2θ).

TABLE 11
XRPD peak positions for Type 8
Peak Position in °2θ

3.1
3.5
4.8
5.6
6.1
9.7
10.7
10.8
11.2
12.7
13.2
13.6
14.2
14.4
15.7
16.5
16.9
17.6
18.3
18.5
19.0
19.1
19.8
20.3
20.7
21.4
22.1
23.3
23.6
24.2
25.1
25.6
25.9
26.6
27.1
27.5
27.9
28.4
28.6
29.2
29.7
30.9
31.4
32.1
32.5
32.8
33.4

In one aspect, the crystalline form Type 8 is characterized by an XRPD pattern measured using Cu Kα (1.5406 Å) radiation substantially the same as shown in FIG. 29.

In one aspect, the crystalline form Type 8 comprises less than 2% by weight of water, such as less than 1.5% or less than 1.0%.

In one aspect, the crystalline form Type 8 is characterized by a DSC thermogram comprising an endothermic event with an onset temperature of 212° C.±2° C. (suitably ±1° C.). In one aspect, the crystalline form Type 8 is characterized by a DSC thermogram comprising no thermal events between 5° and 100° C. and an endothermic event with an onset temperature of 212° C.±2° C. (suitably ±1° C.).

In one aspect, the crystalline form Type 8 is characterized by a DSC thermogram substantially the same as the DSC thermogram depicted in FIG. 30.

In one aspect, the crystalline form Type 8 is at least 75%, 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% free of other forms. In one aspect, the crystalline form Type 8 is free from other forms.

In one aspect, the crystalline form of the present disclosure is stereochemically pure. In one aspect, the crystalline form of the present disclosure is a crystalline form of stereochemically pure Compound I(a):

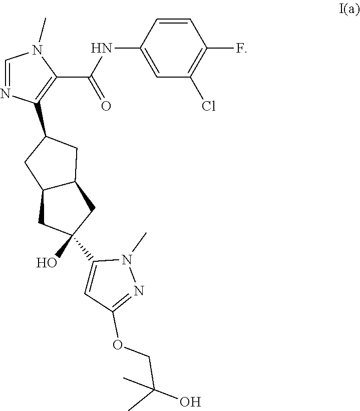 [SMILES: Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

The term “stereochemically pure” in relation to Compound I(a) means that the crystalline form of Compound I(a) is dominated by one diastereomer, for example there is less than about 20% by weight of other diastereomer present (e.g., Compounds I(b), I(c) and/or I(d)), such as less than about 15%, less than about 10%, less than about 5%, less than about 2%, less than about 1%, or less than about 0.5% by weight. Suitably, the crystalline form of Compound I(a) essentially consists of a single diastereomer. Suitably, the crystalline form of Compound I(a) consists of a single diastereomer. In one aspect, the crystalline form of the present disclosure is Compound I(a).

When it is stated herein that the specification relates to a crystalline form of Compound I(a), the degree of crystallinity is conveniently greater than about 60%, more conveniently greater than about 80%, yet more conveniently greater than about 90% and preferably greater than 95%, 98% or 99% by weight.

In one embodiment, Type 1 is pure or substantially pure. As used herein, the term “substantially pure” means that the solid state form of Compound I(a) contains about 20% by weight or less, or about 15% by weight or less, or about 10% by weight or less, or about 5% by weight or less, or about 2% by weight or less, or about 1% by weight or less, or about 0.5% by weight or less of any impurities or other solid forms of Compound I(a), including alternative crystalline forms, hydrates, solvates or amorphous forms, for example as measured, for example by XRPD. Thus, substantially pure Type 1 described herein would be understood to contain greater than about 80% by weight, greater than 85% by weight, greater than 90% by weight, greater than 95% by weight, greater than 98% by weight, greater than 99% by weight, greater than 99.5% by weight of the crystalline Type 1 of Compound I(a). Suitably, there is provided Type 1 wherein when Type 1 is analyzed by a solid-state technique, such as by X-Ray Powder diffraction, no other solid forms (amorphous and/or other crystalline forms) are detected. Suitably, there is provided a crystalline form of Compound I(a) essentially consisting of Type 1. Suitably, there is provided a crystalline form of Compound I(a) consisting of Type 1. Suitably, there is provided a crystalline form of Compound I(a) wherein the form is Type 1 and the form does not comprise Types 2, 3, 4, 5 or 8.

In one embodiment, Type 8 is pure or substantially pure. As used herein, the term “substantially pure” means that the solid state form of Compound I(a) contains about 20% by weight or less, or about 15% by weight or less, or about 10% by weight or less, or about 5% by weight or less, or about 2% by weight or less, or about 1% by weight or less, or about 0.5% by weight or less of any impurities or other solid forms of Compound I(a), including alternative crystalline forms, hydrates, solvates or amorphous forms, for example as measured, for example by XRPD. Thus, substantially pure Type 8 described herein would be understood to contain greater than about 80% by weight, greater than 85% by weight, greater than 90% by weight, greater than 95% by weight, greater than 98% by weight, greater than 99% by weight, greater than 99.5% by weight of the crystalline Type 8 of Compound I(a). Suitably, there is provided Type 8 wherein when Type 8 is analyzed by a solid-state technique, such as by X-Ray Powder diffraction, no other solid forms (amorphous and/or other crystalline forms) are detected. Suitably, there is provided a crystalline form of Compound I(a) essentially consisting of Type 8. Suitably, there is provided a crystalline form of Compound I(a) consisting of Type 8. Suitably, there is provided a crystalline form of Compound I(a) wherein the form is Type 8 and the form does not comprise Type 1, 2, 3, 4, or 5.

In one embodiment, Type 2, 3 or 4 is pure or substantially pure. As used herein, the term “substantially pure” means that the solid state form of Compound I(a) contains about 20% by weight or less, or about 15% by weight or less, or about 10% by weight or less, or about 5% by weight or less, or about 2% by weight or less, or about 1% by weight or less, or about 0.5% by weight or less of any impurities or other solid forms of Compound I(a), including alternative crystalline forms, hydrates, solvates or amorphous forms, for example as measured, for example by XRPD. Thus, substantially pure Type 2, 3 or 4 described herein would be understood to contain greater than about 80% by weight, greater than 85% by weight, greater than 90% by weight, greater than 95% by weight, greater than 98% by weight, greater than 99% by weight, greater than 99.5% by weight of the crystalline Type 2, 3 or 4 of Compound I(a). Suitably, there is provided Type 2, 3 or 4 wherein when Type 2, 3 or 4 is analyzed by a solid-state technique, such as by X-Ray Powder diffraction, no other solid forms (amorphous and/or other crystalline forms) are detected. Suitably, there is provided a crystalline form of Compound I(a) essentially consisting of Type 2, 3 or 4. Suitably, there is provided a crystalline form of Compound I(a) consisting of Type 2, 3 or 4.

One embodiment of the present disclosure includes a pharmaceutical composition comprising a crystalline form or a mixture of crystalline forms according to the present disclosure and a pharmaceutically acceptable carrier, diluent or excipient. Preferably, the pharmaceutical composition comprises a single crystalline form according to the present disclosure and a pharmaceutically acceptable carrier, diluent or excipient.

One embodiment of the present disclosure includes a method of treating Hepatitis B (HBV) infection in a subject in need thereof, the method comprising: administering to the subject a therapeutically effective amount of a crystalline form according to the present disclosure.

One embodiment of the present disclosure includes a method of treating Hepatitis B (HBV) infection in a subject in need thereof, the method comprising: administering to the subject a therapeutically effective amount of a pharmaceutical composition of the present disclosure.

One embodiment includes a crystalline form according to the present disclosure for use in the treatment of HBV infection.

One embodiment includes a pharmaceutical composition of the present disclosure for use in the treatment of HBV infection.

One embodiment includes the use of a crystalline form or a pharmaceutical composition according to the present disclosure for the treatment of HBV infection.

One embodiment includes the use of a crystalline form according to the present disclosure in the manufacture of a medicament for treating HBV infection.

One embodiment includes a process to prepare crystalline Compound I (suitably Compound I(a)), wherein the crystalline form is Type 1, 2, 3, 4, 5 or 8.

In one embodiment, there is provided a process to prepare a crystalline form of Compound I (suitably Compound I(a)), wherein the crystalline form is Type 8.

One embodiment includes a crystalline Compound I (suitably Compound I(a) obtainable by the processes described herein, wherein the crystalline form is Type 1, 2, 3, 4, 5 or 8.

### BRIEF DESCRIPTION OF THE DRAWINGS

FIG. 1 is an XRPD overlay of Types 1, 2, 3, 4, and 5 of the present disclosure.

FIG. 2A illustrates a characterization of Type 1. XRPD showed that Type 1 is crystalline.

FIG. 2B illustrates a characterization of Type 1. The TGA data indicated a two-step weight loss of 1.33% up to 235° C. The DSC exhibited a sharp endotherm at an onset of 210.7° C. PLM showed irregular-shaped birefringent particles.

FIG. 3 is a graphic of a GC of Type 1. GC indicated 0.02% EtOAc and EtOH suggesting residual solvent present in the solids. KF indicated 0.83% water content.

FIG. 4A illustrates a characterization of Type 2. XRPD showed that Type 2 is crystalline.

FIG. 4B illustrate characterization of Type 2. The TGA data indicated a weight loss of 11.6% up to 200° C. The DSC thermogram showed endotherms at 78.1, 93.6, and 212.9° C. (peak temperatures). PLM showed irregularly-shaped birefringent particles.

FIG. 5 illustrates a characterization of Type 2 from slurry at 5° C. using THF: H₂O. Upon drying the TGA indicated a weight loss of 6.8%. Similarly, the KF indicated a water content of 7.10%.

FIG. 6A illustrates a DVS of Type 2 from slurry at 5° C. using THF: H₂O. Type 2 showed water content of 9.2% at 80% RH by DVS, indicating that the sample is a tri-hydrate and is stable between 30-95% RH.

FIG. 6B shows the XPRD diffractograms for Type 2 pre-DVS and for the material acquired post-DVS. Form change was observed post-DVS to a lower order hydrate.

FIG. 7A illustrates a characterization of Type 3. Type 3 was made by keeping Type 2 at 0% RH for 3 days. XRPD showed that Type 3 is crystalline.

FIG. 7B illustrates a characterization of Type 3. The TGA shows a weight loss of 0.75% up to 150° C. The DSC thermogram shows endotherms at 111, 192.4, and 211.5° C. (onset temperatures). PLM showed irregularly-shaped birefringent particles. KF was 3.02% indicating that type 3 is a mono-hydrate. Discrepancy in the KF and TGA weight loss is likely due to the insufficient time given to the sample to equilibrate at RT prior to TGA analysis.

FIG. 8A illustrates a characterization of Type 4. XRPD showed that Type 4 is crystalline.

FIG. 8B illustrates a characterization of Type 4. The TGA showed a weight loss of 6.4% up to 150° C. The DSC thermogram showed endotherms at 84.9° C. corresponding to TGA weight loss and melting event at 209.8° C. (onset temperatures). PLM showed irregularly-shaped birefringent particles.

FIG. 9A is a characterization of Type 5. XRPD showed that Type 5 is crystalline.

FIG. 9B illustrates a characterization of Type 4. The TGA showed a two-step weight loss of 12.1% up to 150° C. The DSC thermogram showed endotherms at 80.6° C. corresponding to TGA weight loss, followed by a recrystallization event at 179.9° C. and concurrent melting at 207.5° C. (onset temperatures). PLM showed irregularly-shaped birefringent particles. KF was 2.26% indicating that Type 5 is a mono-DCM solvate.

FIGS. 10A, 10B and 10C are an illustration of characterization of amorphous material from rotary evaporation. Baseline characterization was conducted on the amorphous form. XRPD showed that the material is amorphous and the DSC thermogram indicated a glass transition at 46.9° C., followed by a recrystallization event at 124.3° C. and a melt at 200.7° C. onset. The TGA indicated a weight loss of 5.8% up to 175° C.

FIG. 11 is an illustration of characterization of slurry conversion experiments from the amorphous form. Shown are XRPD patterns of slurry conversion experiments at RT using amorphous material.

FIGS. 12A and 12B are an illustration of XRPD patterns of temperature cycling experiments using amorphous material.

FIG. 13 is an illustration of XRPD patterns of product resulting from filtered anti-solvent addition experiments using crystalline starting material.

FIGS. 14A and 14B are an illustration of XRPD patterns of product resulting from anti-solvent addition experiments using crystalline starting material.

FIGS. 15A and 15B are an illustration of XRPD patterns of product resulting from slurry at room temperature experiments using crystalline starting material.

FIGS. 16A and 16B are an illustration of XRPD patterns of product resulting from slurry conversion experiments at 60° C. using crystalline starting material.

FIG. 17 is an illustration of XRPD patterns of product from solid vapor diffusion experiments.

FIG. 18 is an illustration of XRPD patterns of product from liquid vapor diffusion experiments.

FIG. 19 is an illustration of XRPD patterns of product from filtered slow cooling experiments.

FIGS. 20A and 20B are an illustration of XRPD patterns of product from slow cooling experiments.

FIG. 21 is an illustration of XRPD patterns of product from slow evaporation experiments.

FIGS. 22A and 22B are an illustration of XRPD patterns of product from temperature cycling experiments.

FIG. 23A is a DVS of Compound I, Type 1. Type 1 showed a water content of 0.67% at 80% RH by DVS, indicating that the sample is slightly hygroscopic.

FIG. 23B show there was no form change was observed post-DVS. KF indicated a water content of 1.02%.

FIG. 24 is an XRPD of Compound I, Type 1, with a characterization of peaks in 20.

FIG. 25 is an XRPD of Compound I, Type 2, with a characterization of peaks in 20.

FIG. 26 is an XRPD of Compound I, Type 3, with a characterization of peaks in 20.

FIG. 27 is an XRPD of Compound I, Type 4, with a characterization of peaks in 20.

FIG. 28 is an XRPD of Compound I, Type 5, with a characterization of peaks in 20.

FIG. 29 is an XRPD of Compound I, Type 8, with a characterization of peaks in 20.

FIG. 30 shows DSC and TGA thermograms of Compound I, Type 8.

FIG. 31 is a DVS of Compound I, Type 8.

FIG. 32 is a PLM of Compound I, Type 8.

FIG. 33 depicts the absolute structure of Compound I.

FIG. 34 is XRPDs of Type 1, Type 8 and material obtained from manufacture.

### DETAILED DESCRIPTION OF THE DISCLOSURE

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

Unless otherwise stated, the following terms used in the specification and claims have the following meanings set out below.

As used herein, “Compound I” refers to N-(3-Chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide.

As used herein, “Compound I(a)” refers to N-(3-chloro-4-fluorophenyl)-4-((2s,3aR,5r,6aS)-5-hydroxy-5-(3-(2-hydroxy-2-methylpropoxy)-1-methyl-1H-pyrazol-5-yl)octahydropentalen-2-yl)-1-methyl-1H-imidazole-5-carboxamide.

As used herein, “API” refers to an active pharmaceutical ingredient, e.g., Compound I.

Unless the context requires otherwise, throughout this specification and claims, the words “comprise,” “comprising” and the like are to be construed in an open, inclusive sense; the words “a” “an” and the like are to be considered as meaning at least one and are not limited to just one; and the term “about” is to be construed as meaning plus or minus 10%. Terms not specifically defined herein should be given the meanings that would be given to them by one of skill in the art in light of the disclosure and the context.

In certain cases, depicted substituents may contribute to optical or stereoisomerism. Compounds having the same molecular formula but differing in the nature or sequence of bonding of their atoms or in the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers”. A single diastereoisomer compound may form one aspect of the present disclosure. More specifically, a solid state form of a single diastereomer compound may form one aspect of the present disclosure.

When a compound has an asymmetric center, for example when it is bonded to four different groups, a pair of enantiomers is possible. An enantiomer can be characterized by the absolute configuration of its asymmetric center and is designated (R) or(S) according to the rules of Cahn and Prelog (Cahn et al., 1966, Angew. Chem. 78:413-447, Angew. Chem., Int. Ed. Engl. 5:385-414 (errata: Angew. Chem., Int. Ed. Engl. 5:511); Prelog and Helmchen, 1982, Angew. Chem. 94:614-631, Angew. Chem. Internat. Ed. Eng. 21:567-583; Mata and Lobo, 1993, Tetrahedron: Asymmetry 4:657-668) or can be characterized by the manner in which the molecule rotates the plane of polarized light and is designated dextrorotatory or levorotatory (namely, as (+)- or (−)-isomers, respectively). A chiral compound can exist as either an individual enantiomer or as a mixture thereof. A mixture containing equal proportions of enantiomers is called a “racemic mixture”.

The compounds of the disclosure may exist as stereoisomers. The term “stereoisomers” when used herein consist of all enantiomers or diastereomers. As noted, these compounds may be designated by the symbols “(+),” “(−),” “R” or “S,” depending on the configuration of substituents around the stereogenic carbon atom. The present disclosure encompasses various stereoisomers of these compounds and mixtures thereof. Mixtures of enantiomers or diastereomers may be designated “(+)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond. Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic ring may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantiomeric and diastereoselective transformations and may involve the use of chiral auxiliaries. For examples, see Carreira and Kvaerno, Classics in Stereoselective Synthesis, Wiley-VCH: Weinheim, 2009.

The compounds of the disclosure may contain one or more double bonds and, therefore, exist as geometric isomers resulting from the arrangement of substituents around a carbon-carbon double bond. The symbol denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond. Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic ring may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The terms “individual,” “patient,” or “subject” are used interchangeably and include any animal, including mammals, preferably mice, rats, other rodents, rabbits, dogs, cats, swine, cattle, sheep, horses, or primates, and most preferably humans. The compounds or pharmaceutical compositions of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, dogs, primates, and the like). The mammal treated in the methods of the disclosure is desirably a mammal in which treatment of HBV infection is desired.

The term “modulation” includes antagonism (e.g., inhibition), agonism, partial antagonism and/or partial agonism.

The term “pharmaceutically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, fillers, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers, diluents or excipients.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The term “therapeutically effective amount” or “effective amount” as used herein refers to the amount of the subject compound that will elicit the biological or medical response of a tissue, system or animal, (e.g., mammal or human) that is being sought by the researcher, veterinarian, medical doctor or other clinician. The compounds or pharmaceutical compositions of the disclosure are administered in therapeutically effective amounts to treat a disease. Alternatively, a therapeutically effective amount of a compound is the quantity required to achieve a desired therapeutic and/or prophylactic effect. The “therapeutically effective amount” will vary depending on the compound, the disease and its severity and the age, weight, etc., of the mammal to be treated.

It is to be appreciated that references to “treat”, “treating” or “treatment” includes: (1) delaying or reducing the likelihood of the appearance of clinical symptoms of the disease or disorder developing in a subject that may be afflicted with the disease or disorder but does not yet experience or display clinical or subclinical symptoms of the disease or disorder, (2) inhibiting the disease or disorder, i.e., arresting, reducing or delaying the progression of the disease or disorder or a relapse thereof (in case of maintenance treatment) or at least one clinical or subclinical symptom thereof, or (3) relieving or attenuating the disease or disorder, i.e., causing regression of the disease, disorder or condition or at least one of its clinical or subclinical symptoms. The term “treating” includes any effect, e.g., lessening, reducing, modulating, or eliminating, via disruption of HBV core protein assembly, that results in the improvement of the disease. “Disruption” includes inhibition of HBV viral assembly and infection. The crystalline compounds disclosed herein can exist in solvated as well as un-solvated forms with pharmaceutically acceptable solvents such as water and the like, and it is intended that the disclosure embrace both solvated and unsolvated forms. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al., Nature Reviews Drug Discovery 2008, 7, 255).

In certain embodiments of the present disclosure, the compounds disclosed herein are “stereochemically pure.” A stereochemically pure compound has a level of stereochemical purity that would be recognized as “pure” by those of skill in the art. Of course, this level of purity may be less than 100%. In certain embodiments, “stereochemically pure” designates a compound that is substantially free, i.e. at least about 85% or more, of alternate isomers. In particular embodiments, the compound is at least about 85%, about 90%, about 91%, about 92%, about 93%, about 94%, about 95%, about 96%, about 97%, about 98%, about 99%, about 99.5% or about 99.9% free of other isomers.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(+)” in nomenclature, but the skilled artisan will recognize that a structure may denote a chiral center implicitly.

At various places in the present specification, values are disclosed in groups or in ranges. It is specifically intended that the description include all individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as,” “including,” or “for example,” is intended merely to illustrate better the present teachings and does not pose a limitation on the scope of the invention unless claimed.

A “crystalline form” is a solid material wherein the constituents of the solid material are arranged in a highly ordered microscopic structure, thereby forming a crystal lattice which extends in all directions. Crystalline forms can include anhydrous crystalline forms, solvated crystalline forms and/or hydrated crystalline forms.

“Polymorphism” is when a solid material can exist in more than one crystalline form.

As used herein, the term “amorphous” refers to a solid material having no long-range order in the position of its molecules. Amorphous solids are substances in which the molecules are arranged in a random manner so that there is no well-defined arrangement, e.g., molecular packing, and no long-range order. Amorphous solids are generally isotropic, i.e., exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid.

A “hydrate” is a compound that exists in a solid composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein. Hydrates may be crystalline, wherein both the compound and water form part of the crystal lattice.

A “solvate” is a similar composition to a hydrate except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein. Solvates may be crystalline, wherein both the compound and solvent form part of the crystal lattice.

“Anhydrous” means the solid form of the compound does not have water incorporated into its structure. For example, an anhydrous crystalline form does not have water forming part of the crystal structure. The skilled person would be aware of techniques which can be used to quantify the amount of water associated with a solid. For example, water content can be determined by either Karl Fischer Titration or Thermogravimetric Analysis (TGA). Suitably, an anhydrous solid form of the compound comprises less than about 2% by weight, such as less than about 1.5%, less than about 1%, such as less than about 0.5, about 0.4, about 0.3, about 0.2, about 0.1, about 0.05, or about 0.01% by weight of water.

“Un-solvated” or “non-solvated” means the solid form of the compound does not have solvent(s) incorporated into its structure. For example, an un-solvated crystalline form does not have solvent(s) forming part of the crystal structure. The skilled person would be aware of techniques which can quantify the amount of solvent associated with a solid. For example, solvent content can be determined by Gas Chromatography (GC). Suitably, an un-solvated or non-solvated solid form of the compound comprises less than about 2% by weight, such as less than about 1.5%, less than about 1%, such as less than about 0.5, about 0.4, about 0.3, about 0.2, about 0.1, about 0.05, or about 0.01% by weight of solvent.

Herein, where a composition is said to “consist essentially of” a particular component, said composition suitably comprises at least 70 wt % of said component, suitably at least 80 wt % thereof, suitably at least 90 wt % thereof, suitably at least 95 wt % thereof, most suitably at least 99 wt % thereof. Suitably, a composition said to “consist essentially of” a particular component consists of said component save for one or more trace components.

The phrase “substantially as shown in figure” refers to an X-ray powder diffraction pattern or DSC thermogram with at least 50%, or at least 60%, or at least 70%, or at least 80%, or at least 90%, or at least 95% or at least 99% of its features appearing in the figure.

The phrase “FIG.” is short for Figure.

### Compounds

Generally, the compounds of the invention can be prepared, isolated or obtained by any method apparent to those of skill in the art. Exemplary methods of preparation are illustrated by the following schemes and description.

Example 55 of PCT/US2021/028323, herein incorporated by reference, provides one embodiment to prepare Compound I: N-(3-Chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide:

### Example 55, PCT '323

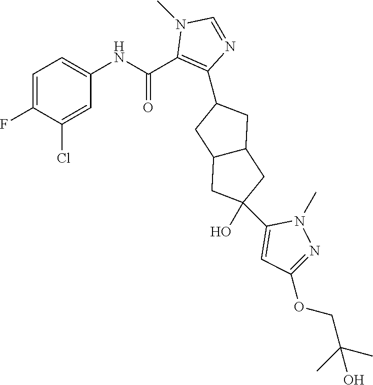 [SMILES: Cn1cnc(C2CC3CC(O)(c4cc(OCC(C)(C)O)nn4C)CC3C2)c1C(=O)Nc1ccc(F)c(Cl)c1]

N-(3-Chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide. MeMgBr (3M in DEE, 0.59 mL, 1.78 mmol) was added slowly to a stirred solution of ethyl 2-((5-(5-(5-((3-chloro-4-fluorophenyl) carbamoyl)-1-methyl-1H-imidazol-4-yl)-2-hydroxyoctahydropentalen-2-yl)-1-methyl-1H-pyrazol-3-yl)oxy)acetate (0.5 g, 0.89 mmol) in dry THF (5 mL) at 0° C. in an inert atmosphere. The reaction mixture was stirred at RT for 2 h. The progress of the reaction was monitored by TLC. After completion, the reaction mixture was quenched with ice cold water and extracted with ethyl acetate. The organic layer was collected; washed with brine; dried over anhydrous sodium sulphate and concentrated under reduced pressure. The crude compound was purified by CombiFlash® column chromatography followed by prep. HPLC to N-(3-chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide (0.501 g, 61%) as an off white solid. TLC: 5% MeOH in DCM (R_{f}: 0.4); ¹H NMR (400 MHz, DMSO-d₆): δ 10.22 (s, 1H), 7.96 (dd, J=6.8 Hz, 2.4 Hz, 1H), 7.65 (s, 1H), 7.59-7.52 (m, 1H), 7.40 (t, J=9.6 Hz, 1H), 5.52 (s, 1H), 5.23 (s, 1H), 4.53 (s, 1H), 3.75-3.70 (m, 5H), 3.67 (s, 3H), 3.26-3.20 (m, 1H), 2.50-2.44 (m, 2H), 2.20-2.06 (m, 4H), 1.90-1.80 (m, 4H), 1.13 (s, 6H) ppm. MS calcd. for C₂₇H₃₃ClFN₅O₄: 545.2; Found: 546.3 [M+1]⁺.

The product of Example 55 may also be referred to herein as Compound I:

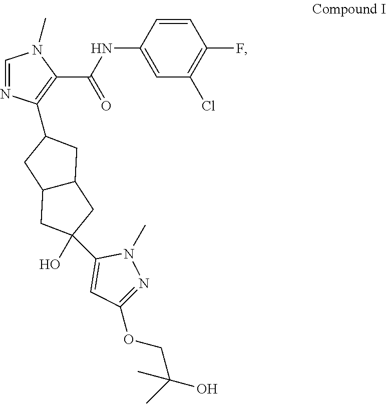 [SMILES: Cn1cnc(C2CC3CC(O)(c4cc(OCC(C)(C)O)nn4C)CC3C2)c1C(=O)Nc1ccc(F)c(Cl)c1]

N-(3-chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide (Compound I). Following alternative naming conventions may provide different chemical names.

As will be appreciated by those skilled in the art, Compound I is a mixture of diastereomers. Therefore, stereoisomerically dominant or stereoisomerically purified compounds, as encompassed within the phrase “stereochemically pure” as used hereinabove, may form one aspect of the present disclosure. The diastereomers of Compound I include:

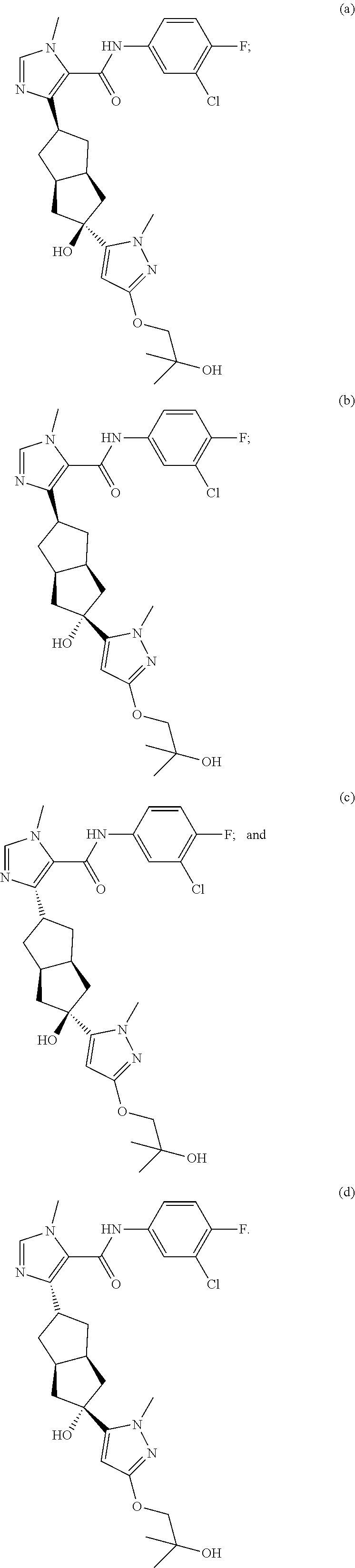

As noted herein, the present disclosure relates to novel solid state forms of Compound I, as well as each of Ia, Ib, Ic, and Id.

Suitably, the present disclosure relates to novel solid state forms of Compound I(a).

### Pharmaceutical Compositions and Kits

In another aspect, the present disclosure provides novel pharmaceutical compositions comprising a crystalline form of Compound I, or a mixture of crystalline forms of Compound I, and a pharmaceutically acceptable carrier, diluent or excipient. Preferably, the pharmaceutical composition comprises a crystalline form of Compound I (suitably a crystalline form of Compound I(a)) and a pharmaceutically acceptable carrier, diluent or excipient. In particular, the present disclosure provides pharmaceutical compositions comprising compounds as disclosed herein formulated together with one or more pharmaceutically acceptable carriers.

Suitably, the pharmaceutical composition comprises a crystalline form of Compound I(a), or a mixture of crystalline forms of Compound I(a), and a pharmaceutically acceptable carrier, diluent or excipient.

These formulations include those suitable for oral, rectal, topical, buccal, parenteral (e.g., subcutaneous, intramuscular, intradermal, or intravenous), rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions of this disclosure may be used in the form of a pharmaceutical preparation, for example, in solid, semisolid or liquid form, which contains one or more compounds of the disclosure, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active ingredient is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another aspect, the disclosure provides enteral pharmaceutical formulations including the Compound I, and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum.

The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric).

The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

Advantageously, the disclosure also provides kits for use by e.g., a consumer in need of HBV infection treatment. Such kits include a suitable dosage form such as those described above and instructions describing the method of using such dosage form to mediate, reduce or prevent HBV infection. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### Methods of Treatment

In a further aspect, a method for treating a hepatitis B infection in a patient in need thereof is provided, comprising administering to a subject or patient a therapeutically effective amount of a crystalline form of a Compound I (suitably a crystalline form of Compound I(a)). In another embodiment, a method for treating a hepatitis B infection in a patient in need thereof is provided, comprising administering to a subject or patient a therapeutically effective amount of a pharmaceutical composition comprising a crystalline form of a Compound I (suitably a crystalline form of Compound I(a)) and a pharmaceutically acceptable carrier, diluent or excipient. In one embodiment, the crystalline form is Type 1, 2, 3, 4, 5 or 8. In one embodiment, the crystalline form is Type 1 or Type 8. Preferably, the crystalline form is Type 8.

In another aspect, there is provided a crystalline form of a Compound I (suitably a crystalline form of Compound I(a)) for use in the treatment of HBV infection. In one embodiment, the crystalline form is Type 1, 2, 3, 4, 5 or 8. In one embodiment, the crystalline form is Type 8. Preferably, the crystalline form is Type 1 or Type 8.

In another aspect, there is provided a pharmaceutical composition comprising a crystalline form of a Compound I (suitably a crystalline form of Compound I(a)), and a pharmaceutically acceptable carrier, diluent or excipient for use in the treatment of HBV infection. In one embodiment, the crystalline form is Type 1, 2, 3, 4, 5 or 8. In one embodiment, the crystalline form is Type 8. Preferably, the crystalline form is Type 1 or Type 8.

In another aspect, there is provided the use of a crystalline form of a Compound I (suitably a crystalline form of Compound I(a)) or the use of a pharmaceutical composition comprising a crystalline form of a Compound I (suitably a crystalline form of Compound I(a)) for the treatment of HBV infection. In one embodiment, the crystalline form is Type 1, 2, 3, 4, 5 or 8. In one embodiment, the crystalline form is Type 8. Preferably, the crystalline form is Type 1 or Type 8.

In another aspect, there is provided the use of a crystalline form of Compound I (suitably a crystalline form of Compound I(a)) or the use of a pharmaceutical composition comprising a crystalline form of a Compound I (suitably a crystalline form of Compound I(a)) in the manufacture of a medicament for treating HBV infection. In one embodiment, the crystalline form is Type 1, 2, 3, 4, 5 or 8. In one embodiment, the crystalline form is Type 8. Preferably, the crystalline form is Type 1 or Type 8.

Pharmaceutical compositions according to the present invention can be dosed via oral, sublingual/buccal, rectal, parenteral, intravenous, intramuscular, subcutaneous, intraventricular, transdermal, topical, inhalation, and/or intranasal administration. In a convenient embodiment, the pharmaceutical compositions according to the present invention are administered orally.

For use in accordance with this aspect, the appropriate dosage is expected to vary depending on, for example, the particular crystalline form of Compound I employed, the mode of administration, and the nature and severity of the infection to be treated as well as the specific infection to be treated and is within the purview of the treating physician. Usually, an indicated administration dose may be in the range between about 0.01 to about 20 mg/kg body weight. In some cases, the administration dose of the compound may be less than 10 mg/kg body weight. In other cases, the administration dose may be less than 5 mg/kg body weight. In yet other cases, the administration dose may be in the range between about 0.01 to about 3 mg/kg body weight.

The dose may be conveniently administered once daily, or in divided doses up to, for example, twice daily or four times a day or in sustained release form. In a convenient embodiment, the pharmaceutical composition is administered daily, such as once daily. In an embodiment, the therapeutically effective amount of a crystalline form of Compound I (suitably a crystalline form of Compound I(a)) is about 5 to 1000 mg (such as about 25-300 mg), administered daily to the subject.

In an embodiment, there is provided a method of treating HBV, wherein the method comprises administering a pharmaceutical composition as described herein, wherein the composition is administered orally with a meal (fed), or at any time between meals (fasted). Conveniently, the composition is administered orally to a subject in a fasted state.

In a convenient embodiment, the unit dosage form of the pharmaceutical composition comprises about 1 mg to about 500 mg (such as about 2 mg to about 400 mg, about 5 mg to about 300 mg, about 5 mg, about 10 mg, or about 50 mg) of a crystalline form of Compound I (suitably a crystalline form of Compound I(a)). In a most convenient embodiment, the unit dosage form of the pharmaceutical composition is a tablet, and the tablet comprises about 1 mg to about 500 mg (such as about 2 mg to about 400 mg, about 5 mg to about 300 mg, about 5 mg, about 10 mg, or about 50 mg) of a crystalline form of Compound I (suitably a crystalline form of Compound I(a)).

In an embodiment, the subject in need of treatment with a crystalline form of Compound I according to the present invention or a pharmaceutical composition comprising a crystalline form of Compound I according to the present invention is treatment naïve and HBeAg (hepatitis B e-antigen) positive prior to treatment. In an embodiment, the subject in need of treatment with a crystalline form of Compound I according to the present invention or a pharmaceutical composition comprising a crystalline form of Compound I according to the present invention is virologically suppressed and HBeAg positive prior to treatment. In an embodiment, the subject in need of treatment with a crystalline form of Compound I according to the present invention or a pharmaceutical composition comprising a crystalline form of Compound I according to the present invention is virologically suppressed and HBeAg negative prior to treatment.

In an embodiment, the subject in need of treatment with a crystalline form of Compound of I according to the present invention or a pharmaceutical composition comprising a crystalline form of Compound I according to the present invention is virologically suppressed for at least 1, 2, 3, 4, 5, or 6 months prior to treatment. In an embodiment, the subject in need of treatment with a crystalline form of Compound I or a pharmaceutical composition comprising a crystalline form of Compound I according to the present invention is virologically suppressed for at least 1, 2, 3, 4, 5, or 6 months prior to treatment and the subject has previously been treated with a nucleos(t)ide inhibitor. In an embodiment, the subject in need of treatment with a crystalline form of Compound I or a pharmaceutical composition comprising a crystalline form of Compound I according to the present invention has previously been treated with a nucleos(t)ide inhibitor for at least 2 months, prior to treatment with a composition of the present invention.

In an embodiment, the crystalline form of Compound I of the present invention or the pharmaceutical composition comprising the crystalline form of Compound I of the present invention is administered to the subject for a treatment period of at least 12 weeks (such as at least 24 weeks, 28 weeks, 32 weeks, 40 weeks, 12 months, 18 months, 24 months or 36 months). In an alternative embodiment, the crystalline form of Compound I of the present invention or the pharmaceutical composition comprising the crystalline form of Compound I of the present invention is administered to the subject until the subject has a reduction in HBeAg and/or HBsAg (hepatitis B surface antigen). In an embodiment, after at least 12 weeks of daily administration of the crystalline form of Compound I of the present invention or the pharmaceutical composition comprising the crystalline form of Compound I of the present invention, the HBeAg positive subject has sustained loss of <0.11 PEI units/mL. In an embodiment, after at least 12 weeks of daily administration of the crystalline form of Compound I of the present invention or the pharmaceutical composition comprising the crystalline form of Compound I of the present invention, the subject has a reduction of HBsAg to ≤100 IU/mL. In an embodiment, after at least 12 weeks of daily administration of the crystalline form of Compound I of the present invention or the pharmaceutical composition comprising the crystalline form of Compound I of the present invention, the subject has a reduction in HBV DNA or HBV RNA.

A compound of the present disclosure may be administered by any conventional route, in particular: enterally, topically, orally, nasally, e.g., in the form of tablets or capsules, via suppositories, or parenterally, e.g., in the form of injectable solutions or suspensions, for intravenous, intra-muscular, sub-cutaneous, or intra-peritoneal injection. Suitable formulations and pharmaceutical compositions will include those formulated in a conventional manner using one or more physiologically acceptable carriers or excipients, and any of those known and commercially available and currently employed in the clinical setting. Thus, the compounds may be formulated for oral, buccal, topical, parenteral, rectal or transdermal administration or in a form suitable for administration by inhalation or insufflation (either orally or nasally).

For oral administration, pharmaceutical compositions may take the form of, for example, tablets or capsules prepared by conventional means with pharmaceutically acceptable excipients including but not limited to one or more of: binding agents (e.g. pregelatinized maize starch, polyvinylpyrrolidone, or hydroxypropyl methylcellulose); fillers (e.g. lactose, microcrystalline cellulose, or calcium hydrogen phosphate); lubricants (e.g. magnesium stearate, talc, or silica); disintegrants (e.g. potato starch or sodium starch glycollate); wetting agents (e.g. sodium lauryl sulphate); pH modifiers (e.g., adipic acid, tartaric acid, sodium hydrogen carbonate, or potassium citrate); complexing agents (e.g., cyclodextrin, phosphates, phosphonates, polycarboxylates, and zeolite); and precipitation inhibitors (Eudragit, PEG, or PEI). Tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). Preparations may also contain buffer salts, flavoring, coloring and sweetening agents as appropriate.

Preparations for oral administration may also be suitably formulated to give controlled-release or sustained release of the active compound(s) over an extended period. For buccal administration the compositions may take the form of tablets or lozenges formulated in a conventional manner known to the skilled artisan.

In a convenient embodiment, the pharmaceutical composition is administered orally.

In a convenient embodiment, the unit dosage form of the pharmaceutical composition is a tablet. Most conveniently, the pharmaceutical composition is a tablet administered orally.

A disclosed compound may also be formulated for parenteral administration by injection e.g., by bolus injection or continuous infusion. Formulations for injection may be presented in unit dosage form e.g., in ampoules or in multi-dose containers, with an added preservative. The compositions may take such forms as suspensions, solutions or emulsions in oily or aqueous vehicles, and may contain additives such as suspending, stabilizing and/or dispersing agents. Alternatively, the compound may be in powder form for constitution with a suitable vehicle, e.g., sterile pyrogen-free water, before use. Compounds may also be formulated for rectal administration as suppositories or retention enemas, e.g., containing conventional suppository bases such as cocoa butter or other glycerides.

Suitably, the crystalline form of Compound (I) is a crystalline form of Compound I(a). In one embodiment, the crystalline form of Compound I(a) is Type 1, 2, 3, 4, 5 or 8. In one embodiment, the crystalline form is Type 8. Preferably, the crystalline form is Type 1 or Type 8.

### Combinations

Also contemplated herein are methods and compositions that include a second active agent, or administering a second active agent. For example, in addition to being infected with HBV, a subject or patient can further have HBV infection-related co-morbidities, i.e., diseases and other adverse health conditions associated with, exacerbated by, or precipitated by being infected with HBV. Contemplated herein are disclosed crystalline forms of the Compound I of the present invention or pharmaceutical compositions comprising a crystalline form of Compound I of the present invention in combination with at least one other agent that has previously been shown to treat these HBV-infection-related conditions.

In some cases, a disclosed compound may be administered as part of a combination therapy in conjunction with one or more antivirals. Example antivirals include nucleoside analogs, interferon α, and other assembly effectors, for instance heteroaryldihydropyrimidines (HAPs) such as methyl 4-(2-chloro-4-fluorophenyl)-6-methyl-2-(pyridin-2-yl)-1,4-dihydropyrimidine-5-carboxylate (HAP-1). For example, provided herein is a method of treating a patient suffering from hepatitis B infection comprising administering to the patient a first amount of a crystalline form of Compound I according to the present invention and a second amount of an antiviral, or other anti HBV agent, for example a second amount of a second compound selected from the group consisting of:
i. HBV capsid assembly promoter (for example, GLS4, BAY 41-4109, AT-130, DVR-23 (e.g., as depicted below),

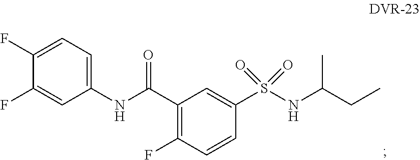 [SMILES: CCC(C)NS(=O)(=O)c1ccc(F)c(C(=O)Nc2ccc(F)c(F)c2)c1]

NVR 3-778, NVR1221 (by code); and N890 (as depicted below):

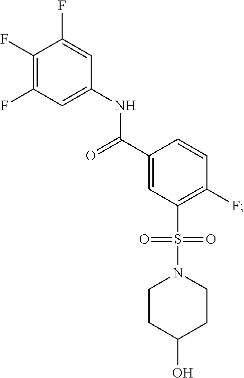 [SMILES: O=C(Nc1cc(F)c(F)c(F)c1)c1ccc(F)c(S(=O)(=O)N2CCC(O)CC2)c1]

ii. Other core protein allosteric modulators (CpAMs) such as those disclosed in the following patent applications hereby incorporated by reference: WO2014037480, WO2014184328, WO2013006394, WO2014089296, WO2014106019, WO2013102655, WO2014184350, WO2014184365, WO2014161888, WO2014131847, WO2014033176, WO2014033167, and WO2014033170;
iii. Nucleos (t) ide analogs interfering with viral polymerase, such as entecavir (Baraclude), Lamivudine, (Epivir-HBV), Telbivudine (Tyzeka, Sebivo), Adefovir dipivoxil (Hepsera), Tenofovir (Viread), Tenofovir alafenamide (Vemlidy), Tenofovir disoproxil fumarate (TDF), Tenofovir alafenamide fumarate (TAF), prodrugs of tenofavir (e.g. AGX-1009), L-FMAU (Clevudine), LB80380 (Besifovir):

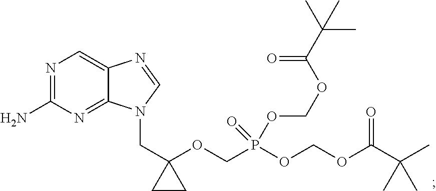 [SMILES: CC(C)(C)C(=O)OCOP(=O)(COC1(Cn2cnc3cnc(N)nc32)CC1)OCOC(=O)C(C)(C)C]

and Active Site Polymerase Inhibitor Nucleotides (ASPINs), such as those disclosed in WO2016099982;
iv. Viral entry inhibitors such as Myrcludex B and related lipopeptide derivatives;
v. HBsAg secretion inhibitors such as REP 9AC′ and related nucleic acid-based amphipathic polymers, HBF-0529 (PBHBV-001), PBHBV-2-15 as depicted below:

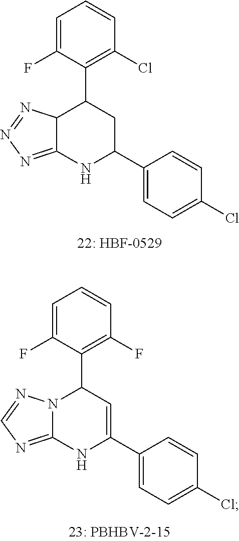 [SMILES: Fc1cccc(Cl)c1C1CC(c2ccc(Cl)cc2)NC2=NN=NC21 ; Fc1cccc(F)c1C1C=C(c2ccc(Cl)cc2)Nc2ncnn21]

and BM601 as depicted below:

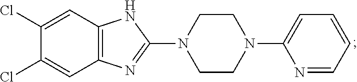 [SMILES: Clc1cc2nc(N3CCN(c4ccccn4)CC3)[nH]c2cc1Cl ; ";"]

vi. Disruptors of nucleocapsid formation or integrity such as NZ-4/W28F:

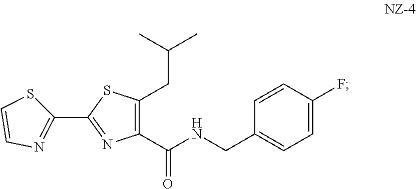 [SMILES: CC(C)Cc1sc(-c2nccs2)nc1C(=O)NCc1ccc(F)cc1]

vii. cccDNA formation inhibitors such as BSBI-25, CCC-0346, CCC-0975 (as depicted below):

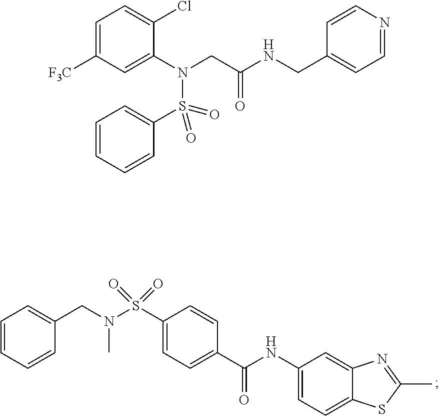 [SMILES: O=C(CN(c1cc(C(F)(F)F)ccc1Cl)S(=O)(=O)c1ccccc1)NCc1ccncc1 ; Cc1nc2cc(NC(=O)c3ccc(S(=O)(=O)N(C)Cc4ccccc4)cc3)ccc2s1 ; ";"]

viii. HBc directed transbodies such as those described in Wang Y, et al, Transbody against hepatitis B virus core protein inhibits hepatitis B virus replication in vitro, Int. Immunopharmacol (2014), located at //dx.doi.org/10.1016/j.intimp.2015.01.028; antiviral core protein mutant (such as Cp183-V124W and related mutations as described in WO/2013/010069, WO2014/074906, each incorporated by reference);
ix. Inhibitors of HBx-interactions such as RNAi, antisense and nucleic acid based polymers targeting HBV RNA, e.g., RNAi (for example ALN-HBV, ARC-520, TKM-HBV, ddRNAi), antisense (ISIS-HBV), or nucleic acid based polymer: (REP 2139-Ca);
x. Immunostimulants such as Interferon alpha 2a (Roferon), Intron A (interferon alpha 2b), Pegasys® (peginterferon alpha 2a), Pegylated IFN 2b, IFN lambda 1a and PEG IFN lambda 1a, Wellferon, Roferon, Infergen, lymphotoxin beta agonists such as CBE11 and BS1);
xi. Non-Interferon Immune enhancers such as Thymosin alpha-1 (Zadaxin) and Interleukin-7 (CYT107);
xii. TLR-7/9 agonists such as GS-9620, CYT003, or Resiquimod;
xiii Cyclophilin inhibitors such as NVP018, OCB-030, SCY-635, Alisporivir, NIM811 and related cyclosporine analogs;
xiv. Vaccines such as GS-4774, TG1050, Core antigen vaccine;
xv. Second mitochondria-derived activator of caspases (SMAC) mimetics such as birinapant and other IAP-antagonists;
xvi. Epigenetic modulators such as KMT inhibitors (EZH1/2, G9a, SETD7, Suv39 inhibitors), PRMT inhibitors, HDAC inhibitors, SIRT agonists, HAT inhibitors, WD antagonists (e.g., OICR-9429), PARP inhibitors, APE inhibitors, DNMT inhibitors, LSD1 inhibitors, JMJD HDM inhibitors, and Bromodomain antagonists;
xvii. Kinase inhibitors such as TKB1 antagonists, PLK1 inhibitors, SRPK inhibitors, CDK2 inhibitors, ATM & ATR kinase inhibitors;
xviii. STING Agonists;
xix. Agents selected from Ribavirin, N-acetyl cysteine, NOV-205 (BAM205), Nitazoxanide (Alinia), Tizoxanide, SB 9200 Small Molecule Nucleic Acid Hybrid (SMNH), DV-601, Arbidol, and FXR agonists (such as GW 4064 and Fexaramin);
xx. Antibodies, therapeutic proteins, gene therapy, and biologics directed against viral components or interacting host proteins.

In some embodiments, the disclosure provides a method of treating a hepatitis B infection in a patient in need thereof, comprising administering a first compound selected from any one of the disclosed crystalline form of Compound I, and one or more other HBV agents each selected from the group consisting of HBV capsid assembly promoters, HBF viral polymerase interfering nucleosides, viral entry inhibitors, HBsAg secretion inhibitors, disruptors of nucleocapsid formation, cccDNA formation inhibitors, antiviral core protein mutant, HBc directed transbodies, RNAi targeting HBV RNA, immunostimulants, TLR-7/9 agonists, cyclophilin inhibitors, HBV vaccines, SMAC mimetics, epigenetic modulators, kinase inhibitors, and STING agonists. In some embodiments, the disclosure provides a method of treating a hepatitis B infection in a patient in need thereof, comprising administering an amount of a disclosed crystalline form of Compound I, and administering another HBV capsid assembly promoter. Suitably, the crystalline form is Type 1, 2, 3, 4, 5 or 8, more suitably Type 8.

In some embodiments, the first and second amounts together comprise a pharmaceutically effective amount. The first amount, the second amount, or both may be the same, more, or less than effective amounts of each compound administered as monotherapies. Therapeutically effective amounts of a disclosed compound and antiviral may be co-administered to the subject, i.e., administered to the subject simultaneously or separately, in any given order and by the same or different routes of administration. In some instances, it may be advantageous to initiate administration of a disclosed compound first, for example one or more days or weeks prior to initiation of administration of the antiviral. Moreover, additional drugs may be given in conjunction with the above combination therapy.

In a convenient embodiment, the method further comprises co-administering to the subject a therapeutically effective amount of a nucleos(t)ide inhibitor. Conveniently, the nucleos(t)ide inhibitor is selected from entecavir, tenofovir, tenofovir alafenamide, and tenofovir disoproxil fumarate. In a convenient embodiment, the nucleos(t)ide inhibitor is administered orally. In a convenient embodiment, the nucleos(t)ide inhibitor is administered daily, such as once daily.

In a convenient embodiment, the method further comprises co-administering to the subject a therapeutically effective amount of pegylated interferon alpha, such as pegylated interferon alpha-2a. In a convenient embodiment, the pegylated interferon alpha is administered by subcutaneous injection. In a convenient embodiment, the pegylated interferon alpha is administered weekly, such as once weekly. In a convenient embodiment, the therapeutically effective amount of the pegylated interferon alpha is about 100 to 300 μg, such as about 180 μg.

In a convenient embodiment, the method further comprises co-administering to the subject a therapeutically effective amount of an siRNA inhibitor of HBV. In a convenient embodiment, the siRNA inhibitor is administered by subcutaneous injection. In a convenient embodiment, the siRNA inhibitor is administered once every 4-12 weeks, such as once every 8 weeks. In a convenient embodiment, the therapeutically effective amount of the siRNA inhibitor is about 20 to 100 mg, such as about 60 mg.

In a convenient embodiment, the method further comprises co-administering to the subject a therapeutically effective amount of an ASPIN. In a convenient embodiment, the ASPIN is administered orally. In a convenient embodiment, the ASPIN is administered daily, such as once daily. In a convenient embodiment, the therapeutically effective amount of the ASPIN is about 10 to 100 mg, such as about 25 mg or about 50 mg.

In an embodiment, there is provided a method of treating hepatitis B in a subject in need thereof, the method comprising administering a therapeutically effective amount of crystalline form of Compound I according to the present invention or a pharmaceutical composition comprising the crystalline form of Compound I according to the present invention, to the subject and co-administering to the subject a therapeutically effective amount of a nucleos(t)ide inhibitor (such as entecavir); and co-administering to the subject a therapeutically effective amount of pegylated interferon alpha (such as pegylated interferon alpha-2a).

In an embodiment, there is provided a method of treating hepatitis B in a subject in need thereof, the method comprising administering a therapeutically effective amount of crystalline form of the Compound I according to the present invention or a pharmaceutical composition comprising the crystalline form of the Compound I according to the present invention, to the subject, and co-administering to the subject a therapeutically effective amount of a nucleos(t)ide inhibitor (such as entecavir); and co-administering to the subject a therapeutically effective amount of an siRNA inhibitor of HBV.

In an embodiment, there is provided a method of treating hepatitis B in a subject in need thereof, the method comprising administering a therapeutically effective amount of crystalline form of the Compound I according to the present invention or a pharmaceutical composition comprising the crystalline form of the Compound I according to the present invention, to the subject, and co-administering to the subject a therapeutically effective amount of a nucleos(t)ide inhibitor (such as entecavir); and co-administering to the subject a therapeutically effective amount of an ASPIN.

Suitably, the crystalline form of Compound (I) is a crystalline form of Compound I(a). In one embodiment, the crystalline form of Compound I(a) is Type 1, 2, 3, 4, 5 or 8. In one embodiment, the crystalline form is Type 8. Preferably, the crystalline form is Type 1 or Type 8.

### Process to Prepare Type 8

In one embodiment, there is provided a process to prepare a crystalline form of Compound I(a) wherein the crystalline form is Type 8.

In one embodiment, the process to prepare Type 8 comprises the steps of:
a) providing a solution of Compound I(a) in a solvent system;
b) cooling the solution from step a) to a temperature of less than 20° C.;
c) stirring the mixture from step b);
d) optionally, isolating the solids formed from step c); and
e) optionally, drying the solids isolated from step d).

In one embodiment, the solvent system comprises a solvent wherein Compound I(a) has a solubility of at least 0.1 mg/mL at room temperature, such as at least 1 mg/mL or at least 2 mg/mL. Suitably, the solvent system comprises a solvent wherein Compound I(a) has a solubility of less than 200 mg/mL at room temperature, such as less than 100 mg/mL, less than 50 mg/mL, less than 20 mg/mL, or less than 10 mg/mL. Suitably, the solvent system comprises ethyl acetate, methanol, ethanol, tetrahydrofuran, dimethyl sulfoxide, acetonitrile, methyl tert-butyl ether and/or water. Suitably, the solvent system comprises ethyl acetate. Suitably, the solvent system consists essentially of ethyl acetate. Suitably, the solvent system consists of ethyl acetate.

In one embodiment, the cooling in step b) is to a temperature of less than 20° C., such as less than 15° C. or 10° C. Suitably, the temperature is between 0 and 15° C., such as between 5 and 10° C.

In one embodiment, the stirring in step c) is performed for at least 1 hour, such as at least 2, 5 or 10 hours. Suitably, the stirring in step c) is performed for between 8 and 30 hours, such as between 10 and 24 hours.

Suitably, step d) comprises isolating the solids by filtration.

Suitably, step e) comprises drying the solids at a temperature greater than room temperature, such as greater than 30° C., or greater than 40° C. Suitably, step e) comprises drying the solids at a temperature of about 50° C.

The invention is illustrated below by the following non-limiting examples.

### Examples

The following abbreviations are used within this specification:
ACN: Acetonitrile
API: Active Pharmaceutical Ingredient
aw: Water activity
DCM: Dichloromethane
DMF: Dimethylformamide
DMSO: Dimethyl sulfoxide
DSC: Differential Scanning calorimetry
eq: Equivalents
EtOAc: Ethyl acetate
h: Hours
KF: Karl Fischer titration
IPA: 2-propanol
MeOH: Methanol
MIBK: Methyl isobutyl ketone
MTBE: t-butyl ether
NMT: not more than
PLM: Polarized light microscopy
PXRD: Powder X-Ray Diffraction
RT: Room Temperature (˜22° C.)
TGA: Thermogravimetric analysis
THF: Tetrahydrofuran
Vol: Volume
XRPD: Powder X-Ray Diffraction

The procedures disclosed herein can be conducted in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. At least some of the compounds identified as intermediates e.g., as part of a synthetic scheme disclosed herein, are contemplated as compounds of the invention.

As previously noted, the present disclosure relates to alternative and novel synthetic methods for the compounds disclosed in PCT/US2021/028323 (PCT '323), which is hereby incorporated by reference in its entirety.

In the procedures described below, it may be necessary to protect reactive functional groups (such as hydroxyl, amino, thio or carboxyl groups) to avoid their unwanted participation in the reactions. The incorporation of such groups, and the methods required to introduce and remove them are known to those skilled in the art (for example, see Greene, Wuts, Protective Groups in Organic Synthesis. 4th Ed. (2007)). The deprotection step may be the final step in the synthesis such that the removal of protecting groups affords compounds of the disclosed process. Starting materials used in the following schemes can be purchased or prepared by methods described in the chemical literature, or by adaptations thereof, using methods known by those skilled in the art. The order in which the steps are performed can vary depending on the groups introduced and the reagents used, but would be apparent to those skilled in the art.

Certain reactions of the disclosed process may be conducted in the presence of a base. Examples of such bases may include, but are not limited to, carbonates such as, e.g., Li₂CO₃, Na₂CO₃, K₂CO₃, Rb₂CO₃, Cs₂CO₃, MgCO₃, CaCO₃, SrCO₃, BaCO₃ and hydrates thereof; and hydroxides such as, e.g., LiOH, NaOH, KOH, Ca(OH)₂, NH₄OH and hydrates thereof; and amines such as methylamine, trimethylamine, trimethylamine, diisopropylethylamine, morpholine and morpholine derivatives.

Certain reactions of the disclosed process involving coupling an amino moiety with a carboxylic acid moiety to form an amide may be conducted in the presence of activator(s). Examples of such activators may include, but are not limited to, carbodiimides such as, e.g., N,N′-dicyclohexylcarbodiimide (DCC), N,N′-diisopropylcarbodiimide (DIC), and carbonyl diimidazole (CDI); and triazoles, such as, e.g., 1-hydroxy-benzotriazole (HOBt) and 1-hydroxy-7-aza-benzotriazole (HOAt). Other activators may include, but are not limited to, e.g., HBTU, HATU, HCTU, TBTU, and PyBOP.

### Analytical Methodology

### XRPD

XRPD was performed with a Panalytical X'Pert³ Powder XRPD on a Si zero-background holder. The 2θ position was calibrated against a Panalytical Si reference standard disc. The parameters used for both crystalline and amorphous material are listed Table 12.

TABLE 12
Parameters for XRPD test

| Parameters | Reflection Mode |
|---|---|
| X-Ray wavelength | Cu, kα |
| | Kα1 (Å): 1.540598, |
| | Kα2 (Å): 1.544426, |
| | Kα2/Kα1 intensity ratio: 0.50 |
| X-Ray tube setting | 45 kV, 40 mA |
| Divergence slit | Fixed ⅛° |
| Scan mode | Continuous |
| Scan range (°2θ) | 3-40 |
| Scan step time [s] | 18.87 |
| Step size (°2θ) | 0.0131 |
| Test Time | 4. min 15 s |

### TGA and DSC:

TGA data was collected using a TA Discovery 550 TGA from TA Instrument. TGA was calibrated using nickel reference standard. DSC was performed using a TA D2500 DSC from TA Instrument. DSC was calibrated with Indium reference standard. Detailed parameters used are listed in Table 13.

TABLE 13
parameters for TGA and DSC test

| Parameters | TGA | DSC |
|---|---|---|
| Method | Ramp | Ramp |
| Sample pan | Platinum, open | Aluminum, crimped |
| Temperature | RT - desired temperature | 25° C. - desired temperature |
| Heating rate | 10° C./min | 10° C./min |
| Purge gas | N₂ | N₂ |

### PLM

Polarized light microscopic picture was captured on Nikon DS-Fi2 upright microscope at room temperature.

### KF

Karl Fischer titration (KF) was collected on C30S coulometric KF titrator with the DO308 Oven (Mettler Toledo) to determine the content of water in the samples. Oven water standard 5.55% was used as a standard to verify the coulometric KF titrator with the D0308 Oven.

### HPLC Method

The HPLC method conditions used for measuring solubility and stability samples are summarized in Table 14.

TABLE 14
HPLC method

| HPLC | Thermofisher Vanquish HPLC | | |
|---|---|---|---|
| Mobile Phase A | 5 mM of NH₄H₂PO₄ + 5 mM of (NH₄)₂HPO₄ in water | | |
| Mobile Phase B | ACN:MeOH = 6:4 (v/v) | | |
| Column | XBridge C18 (4.6 mm*150 mm*3.5 μm) PN | | |
| | 186003034 | | |
| Column | 40° C. | | |
| Temperature | | | |
| Injector Volume | 5 mL | | |
| Detector | 210 nm | | |
| Wavelength | | | |
| Flow Rate | 1.0 mL/min | | |
| Diluent | MeOH | | |
| | Time | % Mobile | % Mobile |
| | (min) | Phase A | Phase B |
| Gradient | 0 | 95 | 5 |
| Program | 4 | 50 | 50 |
| | 10 | 40 | 60 |
| | 14 | 15 | 85 |
| | 17 | 15 | 85 |
| | 18 | 95 | 5 |
| | 25 | 95 | 5 |

### Example 1: Synthesis of Compound I

Example 55 of PCT/US2021/028323, herein incorporated by reference, provides one embodiment to prepare a racemate, Compound I: N-(3-Chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide:

### Example 55, PCT '323

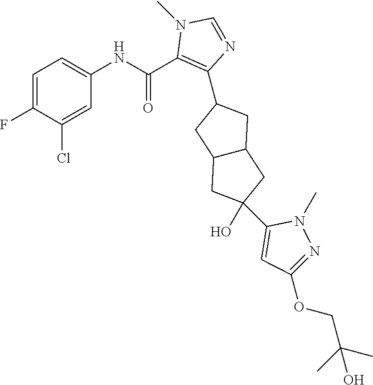 [SMILES: Cn1cnc(C2CC3CC(O)(c4cc(OCC(C)(C)O)nn4C)CC3C2)c1C(=O)Nc1ccc(F)c(Cl)c1]

### General Procedure for Alkylation, Method A

To a stirred solution of Ar—OH (1 eq.) and a halo compound (2 eq.) in acetonitrile/DMF (4 mL/mmol) was added K₂CO₃ (2 eq.) and KI (0.5 eq.). The reaction mixture was stirred at 60° C.-80° C. for 1216 h. The reaction progress was monitored by TLC. After completion, the reaction mixture was diluted with water and extracted with ethyl acetate. The combined organic layers were collected, dried over anhydrous sodium sulphate, and concentrated under reduced pressure to obtain crude compound which was purified by silica gel column chromatography or prep-HPLC to afford the desired compound.

### General Procedure for Alkylation, Method B

To a stirred solution of Ar—OH (1 eq.) and a halo compound (2 eq) in DMF/ACN (6 mL/mmol) was added Cs₂CO₃ (2.5 eq.). The reaction mixture was stirred at RT/60° C. for 2-4 h. The reaction progress was monitored by TLC. After completion, the reaction mixture was diluted with water and extracted with ethyl acetate. The combined organic layers were collected, dried over anhydrous sodium sulphate, and concentrated under reduced pressure to obtain crude compound which was purified by silica gel column chromatography or prep-HPLC to afford the desired compound.

### Intermediate 20 of PCT '323

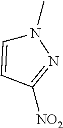

1-Methyl-3-nitro-1H-pyrazole. NaOtBu (19.11 g, 199.1 mmol) was added to a stirred solution of 3-nitro-1H-pyrazole (15 g, 132.7 mmol) in DMF (150 mL) at 0° C., and the reaction was stirred for 20 minutes. Mel (9.91 mL 159.24 mmol) was then added dropwise. The resulting mixture was stirred at RT for 16 h. The progress of the reaction was monitored by TLC. After completion, the reaction mixture was quenched with water and extracted with ethyl acetate. The organic layer was collected; washed with brine; dried over anhydrous sodium sulphate and concentrated under reduced pressure. The crude compound was purified by silica gel column chromatography to afford 1-methyl-3-nitro-1H-pyrazole (10 g, 59%) as an off white solid. TLC: 20% EtOAc/hexane (R_{f}: 0.2). ¹H NMR (400 MHz, DMSO-d₆): δ 7.98 (s, 1H), 7.03 (d, J=2.0 Hz, 1H), 3.97 (s, 3H) ppm.

### Example 50 of PCT '323

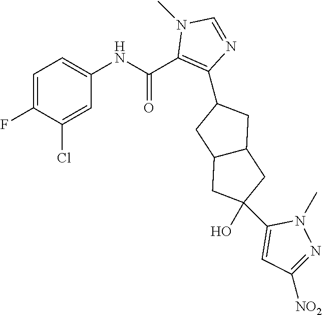 [SMILES: Cn1cnc(C2CC3CC(O)(c4cc([N+](=O)[O-])nn4C)CC3C2)c1C(=O)Nc1ccc(F)c(Cl)c1]

N-(3-Chloro-4-fluorophenyl)-4-(5-hydroxy-5-(1-methyl-3-nitro-1H-pyrazol-5-yl)octahydropentalen-2-yl)-1-methyl-1H-imidazole-5-carboxamide. LDA (2M in THF, 60 mL, 120 mmol) was added dropwise to a stirred solution of methyl-3-nitro-1H-pyrazole (10.16 g, 80 mmol) in dry THF (100 mL) at −78° C. under an inert atmosphere and the reaction mixture stirred for 2 h. To this was added a solution of N-(3-chloro-4-fluorophenyl)-1-methyl-4-(5-oxooctahydropentalen-2-yl)-1H-imidazole-5-carboxamide (3 g, 8 mmol) in THF at −78° C. The resulting reaction mixture was stirred at −78° C. for 1 h. The progress of the reaction was monitored by TLC and LCMS. After completion, the reaction was quenched with a saturated NH₄Cl solution and extracted with ethyl acetate. The organic layer was collected; washed with brine; dried over anhydrous sodium sulphate and concentrated under reduced pressure. The crude compound was purified by silica gel column chromatography to afford N-(3-chloro-4-fluorophenyl)-4-5-hydroxy-5-(1-methyl-3-nitro-1H-pyrazol-5-yl)octahydropentalen-2-yl)-1-methyl-1H-imidazole-5-carboxamide as a single diastereomer (2 g, 50%) as an off white solid. TLC: 5% MeOH/DCM (R_{f}: 0.3). ¹H-NMR (DMSO-d6, 400 MHz): δ 10.23 (s, 1H), 7.96 (dd, J=6.8 Hz, 2.4 Hz, 1H), 7.66 (s, 1H), 7.59-7.55 (m, 1H), 7.40 (t, J=9.2 Hz, 1H), 6.93 (s, 1H), 5.60 (s, 1H), 4.05 (s, 3H), 3.68 (s, 3H), 3.29-3.24 (m, 1H), 2.51-2.49 (m, 2H), 2.30-2.24 (m, 2H), 2.13-2.07 (m, 2H), 1.94-1.85 (m, 4H) ppm; MS calcd. for C₂₃H₂₄ClFN₆O₄: 502.2; Found: 503.3 [M+1]⁺.

### Example 51 of PCT '323

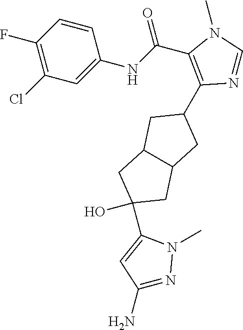 [SMILES: Cn1cnc(C2CC3CC(O)(c4cc(N)nn4C)CC3C2)c1C(=O)Nc1ccc(F)c(Cl)c1]

4-(5-(3-Amino-1-methyl-1H-pyrazol-5-yl)-5-hydroxyoctahydropentalen-2-yl)-N-(3-chloro-4-fluorophenyl)-1-methyl-1H-imidazole-5-carboxamide. 10% Pd/C (0.5 g) and NaBH₄ (1.06 g, 27.88 mmol) were added to a stirred solution of N-(3-chloro-4-fluorophenyl)-4-5-hydroxy-5-(1-methyl-3-nitro-1H-pyrazol-5-yl)octahydropentalen-2-yl)-1-methyl-1H-imidazole-5-carboxamide (2 g, 3.98 mmol) in MeOH (20 mL) under a nitrogen atmosphere. The reaction mixture was stirred at 0° C. for 30 minutes. The progress of the reaction was monitored by TLC and LCMS. After completion, the reaction mixture was filtered through a pad of Celite and washed with methanol. The filtrate was concentrated under reduced pressure. The residue was diluted with water and extracted with ethyl acetate. The organic layer was collected; washed with brine; dried over anhydrous sodium sulphate and concentrated under reduced pressure. The crude compound was purified by silica gel column chromatography to afford 4-5-(3-amino-1-methyl-1H-pyrazol-5-yl)-5-hydroxyoctahydropentalen-2-yl)-N-(3-chloro-4-fluorophenyl)-1-methyl-1H-imidazole-5-carboxamide (1.5 g, 80%) as an off white solid. TLC: 10% MeOH/DCM (R_{f}: 0.1). ¹H NMR (400 MHz, DMSO-d₆): δ 10.25 (s, 1H), 7.95 (d, J=4.4 Hz, 1H), 7.76 (s, 1H), 7.58-7.54 (m, 1H), 7.41 (t, J=9.2 Hz, 1H), 6.62-5.57 (br s, 2H), 5.39 (s, 1H), 5.17 (s, 1H), 3.69 (s, 6H), 3.32-3.31 (m, 1H, merged), 2.50-2.32 (m, 2H, merged), 2.29-2.11 (m, 4H), 1.85-1.83 (m, 4H) ppm; MS calcd. for C₂₃H₂₆ClFN₆O₂; 472.2; Found: 471.2 [M−1]⁻.

### Example 52 of PCT '323

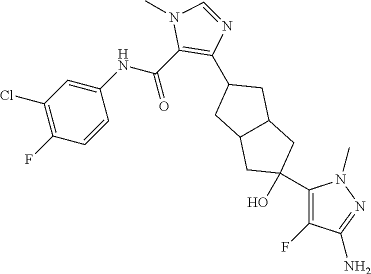 [SMILES: Cn1cnc(C2CC3CC(O)(c4c(F)c(N)nn4C)CC3C2)c1C(=O)Nc1ccc(F)c(Cl)c1]

4-(5-(3-Amino-4-fluoro-1-methyl-1H-pyrazol-5-yl)-5-hydroxyoctahydropentalen-2-yl)-N-(3-chloro-4-fluorophenyl)-1-methyl-1H-imidazole-5-carboxamide. Selectfluor (0.149 g, 0.42 mmol) and DIPEA (0.147 mL, 0.84 mmol) were added to a stirred solution of 4-(5-(3-amino-1-methyl-1H-pyrazol-5-yl)-5-hydroxyoctahydropentalen-2-yl)-N-(3-chloro-4-fluorophenyl)-1-methyl-1H-imidazole-5-carboxamide (0.2 g, 0.42 mmol) in ACN (5 mL). The reaction mixture was stirred at 100° C. for 16 h. The progress of the reaction was monitored by TLC and LCMS. After completion, the reaction mixture was concentrated under reduced pressure. The crude compound was purified by silica gel column chromatography to afford 4-(5-(3-amino-4-fluoro-1-methyl-1H-pyrazol-5-yl)-5-hydroxyoctahydropentalen-2-yl)-N-(3-chloro-4-fluorophenyl)-1-methyl-1H-imidazole-5-carboxamide (0.02 g, 10%) as an off white solid. TLC: 10% MeOH in DCM (R_{f}: 0.3). ¹H NMR (400 MHz, DMSO-d₆): δ 10.19 (s, 1H), 7.95 (dd, J=6.8, 2.4 Hz, 1H), 7.63 (s, 1H), 7.57-7.53 (m, 1H), 7.39 (t, J=9.6 Hz, 1H), 5.21 (s, 1H), 4.47 (s, 2H), 3.66 (s, 3H), 3.60 (s, 3H), 3.30-3.14 (m, 1H), 2.50-2.40 (m, 2H, merged), 2.23-2.16 (m, 2H), 2.07-2.04 (m, 2H), 1.96-1.83 (m, 4H). MS calcd. for C₂₃H₂₅ClF₂N₆O₂: 490.2; Found: 473.1 [M−H₂O+1]⁺.

### Intermediate 21 of PCT '323

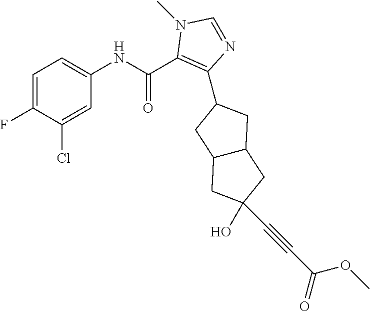 [SMILES: COC(=O)C#CC1(O)CC2CC(c3ncn(C)c3C(=O)Nc3ccc(F)c(Cl)c3)CC2C1]

Methyl 3-(5-(5-((3-chloro-4-fluorophenyl) carbamoyl)-1-methyl-1H-imidazol-4-yl)-2-hydroxyoctahydropentalen-2-yl) propiolate. n-BuLi (1.19 g, 18.6 mmol) was added to a stirred solution of methyl propiolate (1.56 g, 18.6 mmol) in dry THF (40 mL) at −78° C. in an inert atmosphere and the reaction mixture was stirred for 30 minutes. To this a solution of N-(3-chloro-4-fluorophenyl)-1-methyl-4-(5-oxooctahydropentalen-2-yl)-1H-imidazole-5-carboxamide (1 g, 2.66 mmol) in THF was added at −78° C. The resulting reaction mixture was stirred at −78° C. for 2 h. The progress of the reaction was monitored by TLC and LCMS. After completion, the reaction mixture was quenched with saturated NH₄Cl solution and extracted with ethyl acetate. The organic layer was collected; washed with brine; dried over anhydrous sodium sulphate and concentrated under reduced pressure. The crude compound was purified by silica gel column chromatography to afford methyl 3-(5-(5-((3-chloro-4-fluorophenyl) carbamoyl)-1-methyl-1H-imidazol-4-yl)-2-hydroxyoctahydropentalen-2-yl) propiolate, an off white solid, as a single diastereomer. TLC: 5% MeOH/DCM (R_{f}: 0.3); ¹H NMR (400 MHz, DMSO-d₆): δ 10.23 (s, 1H), 7.95 (d, J=6.4 Hz, 1H), 7.74-7.68 (m, 1H), 7.59-7.55 (m, 1H), 7.40 (t, J=8.8 Hz, 1H), 5.79 (s, 1H), 3.69 (s, 3H), 3.63 (s, 3H), 3.28-3.23 (m, 1H), 2.58-2.54 (m, 2H), 2.09-2.06 (m, 4H), 1.80-1.76 (m, 4H) ppm. MS calcd. for C₂₃H₂₃ClFN₃O₄: 459.1; Found: 460.2 [M+1]⁺.

### Example 53 of PCT '323

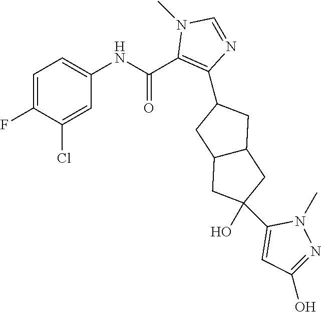 [SMILES: Cn1cnc(C2CC3CC(O)(c4cc(O)nn4C)CC3C2)c1C(=O)Nc1ccc(F)c(Cl)c1]

N-(3-Chloro-4-fluorophenyl)-4-(5-hydroxy-5-(3-hydroxy-1-methyl-1H-pyrazol-5-yl)octahydropentalen-2-yl)-1-methyl-1H-imidazole-5-carboxamide. TEA (2 g, 19.82 mmol) and 3-(5-(5-((3-Chloro-4-fluorophenyl) carbamoyl)-1-methyl-1H-imidazol-4-yl)-2-hydroxyoctahydropentalen-2-yl) propiolate (1.3 g, 2.83 mmol) were added to a stirred solution of methyl hydrazine sulphate (2.85 g, 19.82 mmol) in EtOH (20 mL). The reaction mixture was stirred at 50° C. for 24 h. The progress of the reaction was monitored by TLC and LCMS. After completion, the reaction mixture was concentrated under reduced pressure. The residue was diluted with water and extracted with ethyl acetate. The organic layer was collected; washed with brine; dried over anhydrous sodium sulphate and concentrated under reduced pressure to afford N-(3-chloro-4-fluorophenyl)-4-(5-hydroxy-5-(3-hydroxy-1-methyl-1H-pyrazol-5-yl)octahydropentalen-2-yl)-1-methyl-1H-imidazole-5-carboxamide (0.65 g, 49%) as a white solid. TLC: 8% MeOH/DCM (R_{f}: 0.2); ¹H NMR (400 MHz, DMSO-d₆): δ 10.18 (s, 1H), 9.27 (s, 1H), 7.95 (d, J=4.4 Hz, 1H), 7.64 (s, 1H), 7.61-7.55 (m, 1H), 7.39 (t, J=8.8 Hz, 1H), 5.28 (s, 1H), 5.13 (s, 1H), 3.66 (s, 6H), 3.38-3.18 (m, 1H, merged), 2.60-2.38 (m, 2H, merged), 2.20-2.01 (m, 4H), 1.91-1.75 (m, 4H) ppm. MS calcd. for C₂₃H₂₅ClFN₅O₃: 473.2; Found: 473.9 [M+1]⁺.

### Example 54 of PCT '323

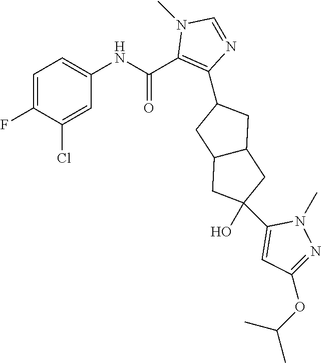 [SMILES: CC(C)Oc1cc(C2(O)CC3CC(c4ncn(C)c4C(=O)Nc4ccc(F)c(Cl)c4)CC3C2)n(C)n1]

N-(3-Chloro-4-fluorophenyl)-4-(5-hydroxy-5-(3-isopropoxy-1-methyl-1H-pyrazol-5-yl)octahydropentalen-2-yl)-1-methyl-1H-imidazole-5-carboxamide. The title compound was synthesized by alkylation of N-(3-chloro-4-fluorophenyl)-4-(5-hydroxy-5-(3-hydroxy-1-methyl-1H-pyrazol-5-yl)octahydropentalen-2-yl)-1-methyl-1H-imidazole-5-carboxamide using method A. ¹H NMR (400 MHz, DMSO-d₆): δ 10.21 (s, 1H), 7.99-7.94 (m, 1H), 7.64 (s, 1H), 7.60-7.54 (m, 1H), 7.40 (t, J=9.2 Hz, 1H), 5.47 (s, 1H), 5.20 (s, 1H), 4.61-4.54 (m, 1H), 3.71 (s, 3H), 3.67 (s, 3H), 3.29-3.18 (m, 1H), 2.48-2.39 (m, 2H), 2.20-2.04 (m, 4H), 1.90-1.78 (m, 4H), 1.21 (d, J=6.4 Hz, 6H) ppm; TLC: 10% MeOH/DCM (R_{f}: 0.3); MS calcd. for C₂₆H₃₁ClFN₅O₃: 515.2; Found: 516.1 [M+1]⁺.

### Example 55 of PCT '323

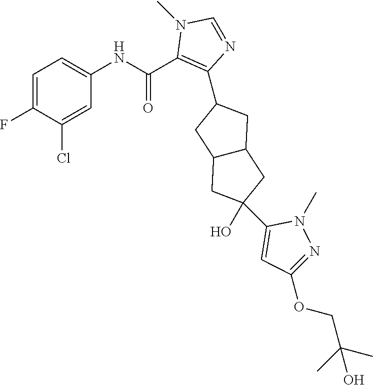 [SMILES: Cn1cnc(C2CC3CC(O)(c4cc(OCC(C)(C)O)nn4C)CC3C2)c1C(=O)Nc1ccc(F)c(Cl)c1]

N-(3-Chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide. MeMgBr (3M in DEE, 0.59 mL, 1.78 mmol) was added slowly to a stirred solution of ethyl 2-((5-(5-(5-((3-chloro-4-fluorophenyl) carbamoyl)-1-methyl-1H-imidazol-4-yl)-2-hydroxyoctahydropentalen-2-yl)-1-methyl-1H-pyrazol-3-yl)oxy)acetate (0.5 g, 0.89 mmol) in dry THF (5 mL) at 0° C. in an inert atmosphere. The reaction mixture was stirred at RT for 2 h. The progress of the reaction was monitored by TLC. After completion, the reaction mixture was quenched with ice cold water and extracted with ethyl acetate. The organic layer was collected; washed with brine; dried over anhydrous sodium sulphate and concentrated under reduced pressure. The crude compound was purified by CombiFlash® column chromatography followed by prep. HPLC to N-(3-chloro-4-fluorophenyl)-4-(5-hydroxy-5-(3-(2-hydroxy-2-methylpropoxy)-1-methyl-1H-pyrazol-5-yl)octahydropentalen-2-yl)-1-methyl-1H-imidazole-5-carboxamide (0.501 g, 61%) as an off white solid. TLC: 5% MeOH in DCM (R_{f}: 0.4); ¹H NMR (400 MHz, DMSO-d₆): δ 10.22 (s, 1H), 7.96 (dd, J=6.8 Hz, 2.4 Hz, 1H), 7.65 (s, 1H), 7.59-7.52 (m, 1H), 7.40 (t, J=9.6 Hz, 1H), 5.52 (s, 1H), 5.23 (s, 1H), 4.53 (s, 1H), 3.75-3.70 (m, 5H), 3.67 (s, 3H), 3.26-3.20 (m, 1H), 2.50-2.44 (m, 2H), 2.20-2.06 (m, 4H), 1.90-1.80 (m, 4H), 1.13 (s, 6H) ppm. MS calcd. for C₂₇H₃₃ClFN₅O₄: 545.2; Found: 546.3 [M+1]⁺.

### Scheme

As previously noted, the present disclosure relates to alternative and novel synthetic methods for the compounds disclosed in PCT/US2021/028323 (PCT '323), which is hereby incorporated by reference in its entirety.

One route to obtain Compound I uses the guidance of PCT '323 as a template:

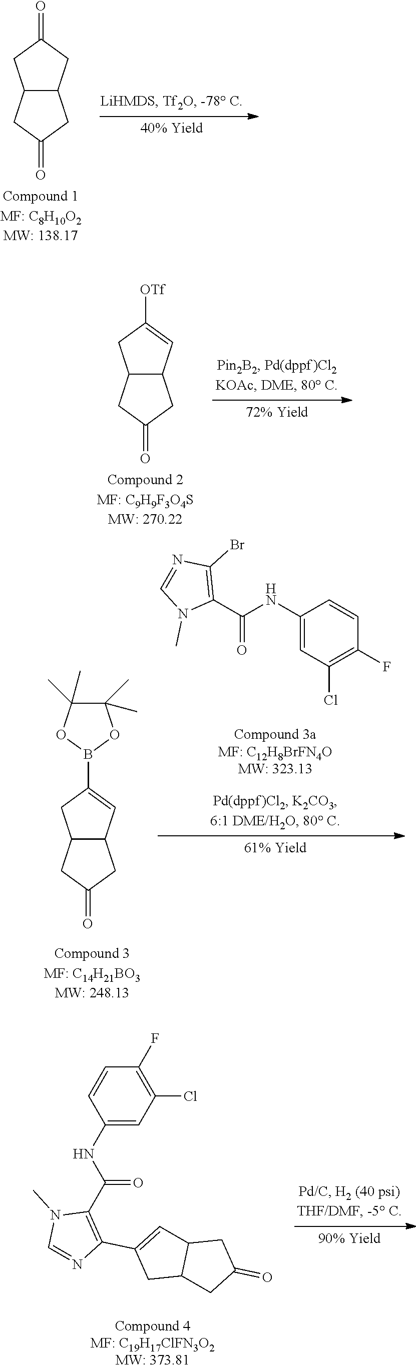

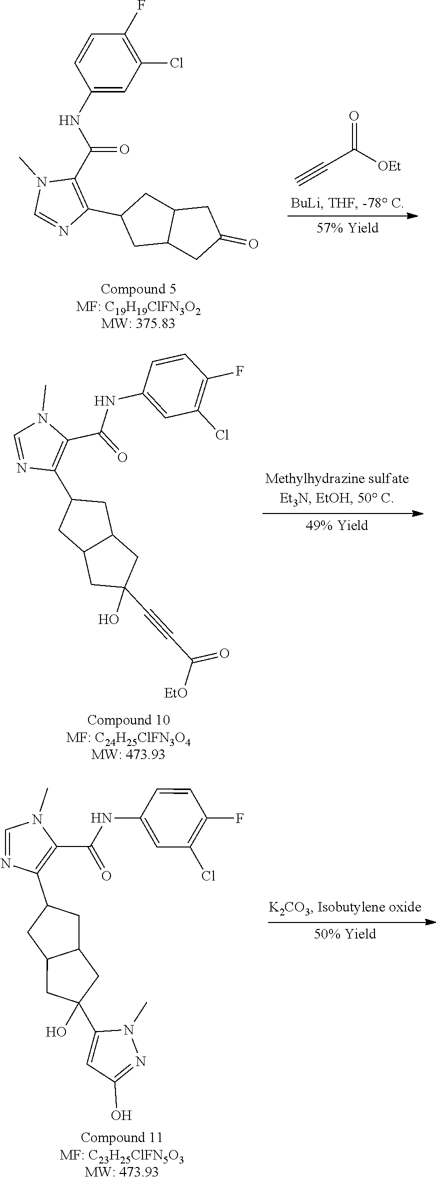

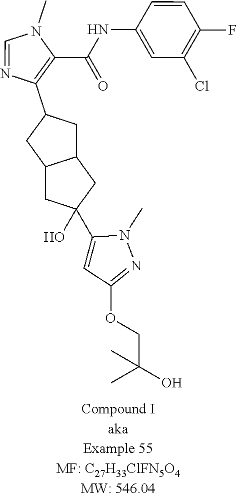 [SMILES: Cn1cnc(C2CC3CC(O)(c4cc(OCC(C)(C)O)nn4C)CC3C2)c1C(=O)Nc1ccc(F)c(Cl)c1]

Alternative routes exist, including that which is disclosed in co-pending U.S. Provisional Application No. 63/257,697, filed Oct. 20, 2021, and herein incorporated by reference in its entirety.

### Example 2

### Generation of Crystalline Forms

One embodiment of the present disclosure is novel solid state forms of Compound I. A number of crystallization experiments were conducted using different techniques (slurry at RT and 60° C., slow evaporation, slow cooling, anti-solvent addition, slurry thermal cycling, solid vapor diffusion and liquid vapor diffusion) in a variety of solvent systems with crystalline staring material or amorphous material. Only one anhydrous form (starting API) was identified. This form was assigned as Type 1. A tri-hydrate form (Type 2) was obtained in THF/water and acetone/water systems. After desorption and resorption, Type 2 converted to Type 3. Type 3 was determined as a monohydrate form. Another hydrated form (Type 4) was identified in a slurry study in MTBE. A DCM solvate form (Type 5) was generated in a solid vapor diffusion study in DCM.

In one embodiment, one or more form is created by slurry at RT or 60° C.: excess amount of API was suspended in selected solvents for 3 days. If API was completely dissolved at the end of the study, sample was transferred for slow evaporation.

In one embodiment, one or more form is created by slow cooling: excess amount of API was suspended in selected solvents at 50° C. Suspension was filtered. Filtrate was stored at refrigerated condition. If no crystal was formed at the end of the study, sample was transferred for slow evaporation.

In one embodiment, one or more form is created by slow evaporation: a nearly saturated solution was prepared in different solvents. Solution was filtered and filtrate was placed at ambient environment.

In one embodiment, one or more form is created by anti-solvent addition: API was dissolved in selected solvents. Solution was filtrated and antisolvent was introduced slowly.

In one embodiment, one or more form is created by liquid-vapor diffusion: study was set up in a two-vial system (a smaller vial sitting inside a bigger vial). Filtered API solution was put into the smaller vial while antisolvent was in the larger vial.

In one embodiment, one or more form is created by solid-vapor diffusion: study was set up in a two-vial system (a smaller vial sitting inside a bigger vial). API was put into the smaller vial while solvent was in the larger vial.

In one embodiment, one or more form is created by thermal cycling: excess amount of API was suspended in selected solvents. Suspension was then subjected to alternating thermal program between 5° C. and 50° C.

As will be indicated, for slurry experiments, thermal cycling and solid vapor experiments, both crystalline API (Type 1) and amorphous API were used.

These techniques are known by those skilled in the art.

### Characterization of Crystalline Forms

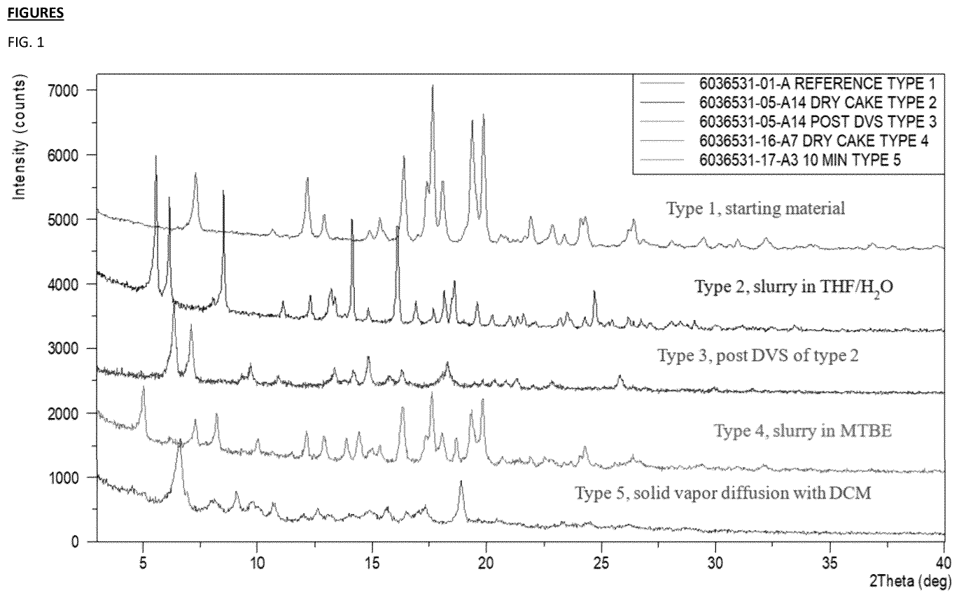

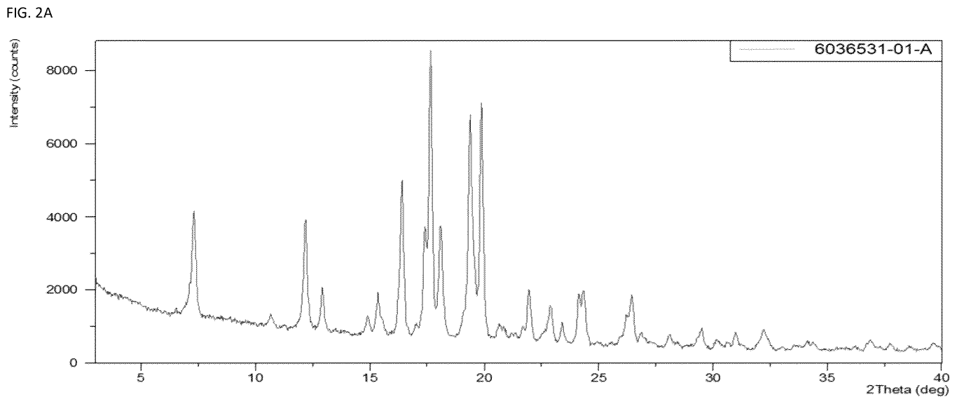

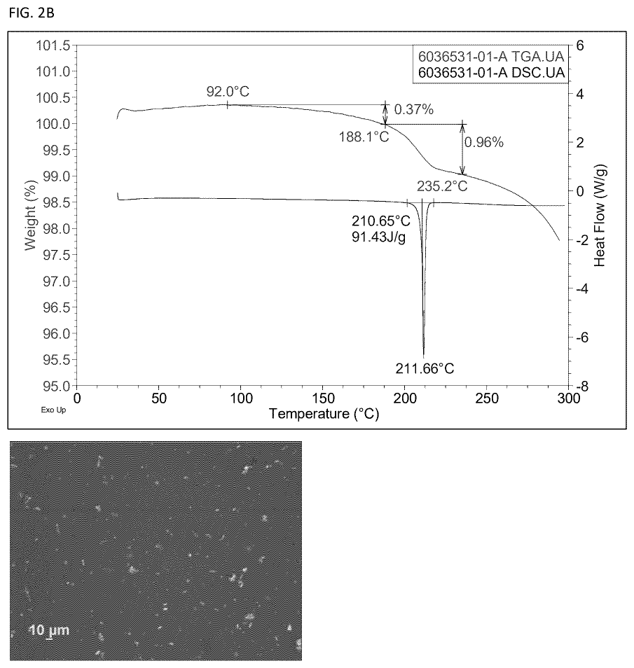

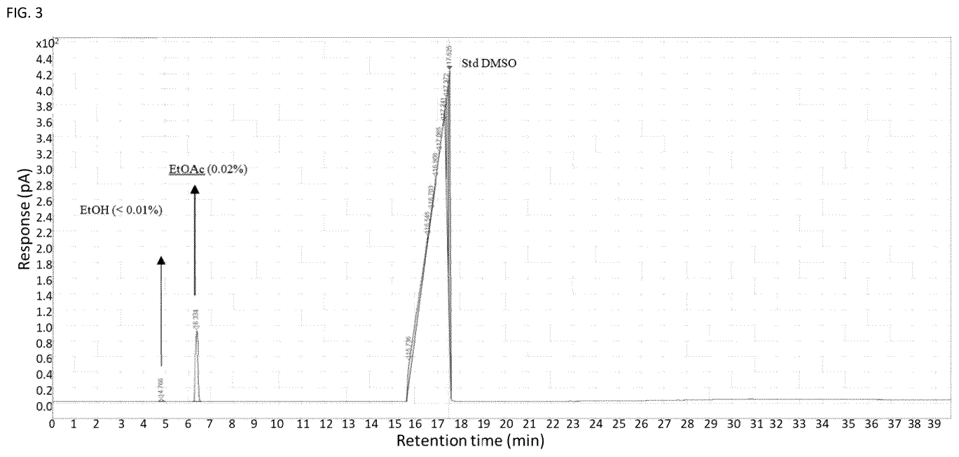

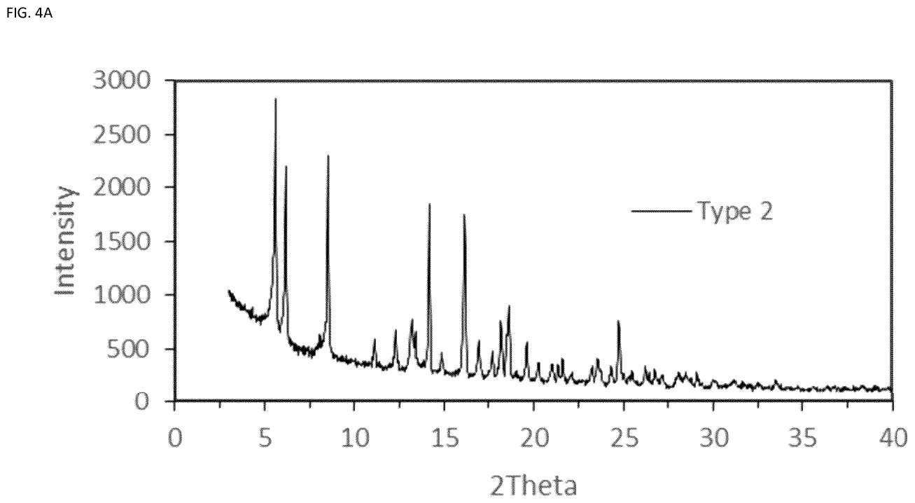

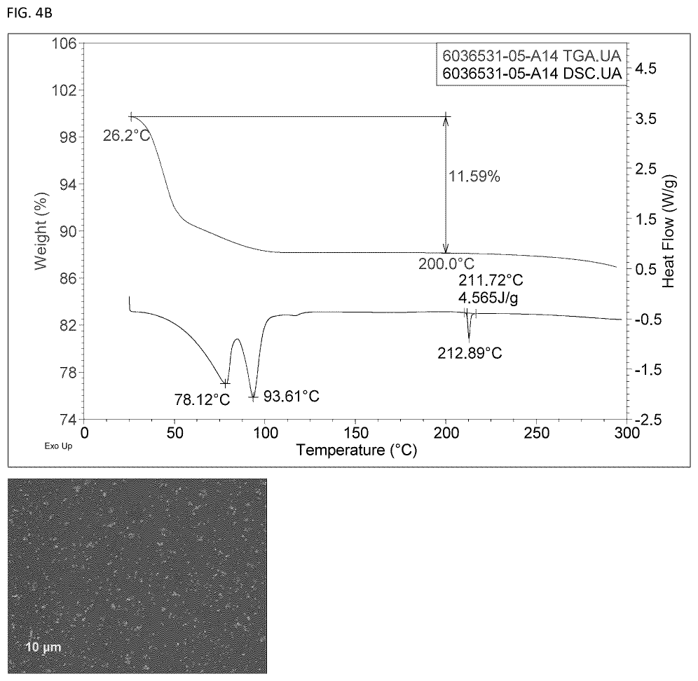

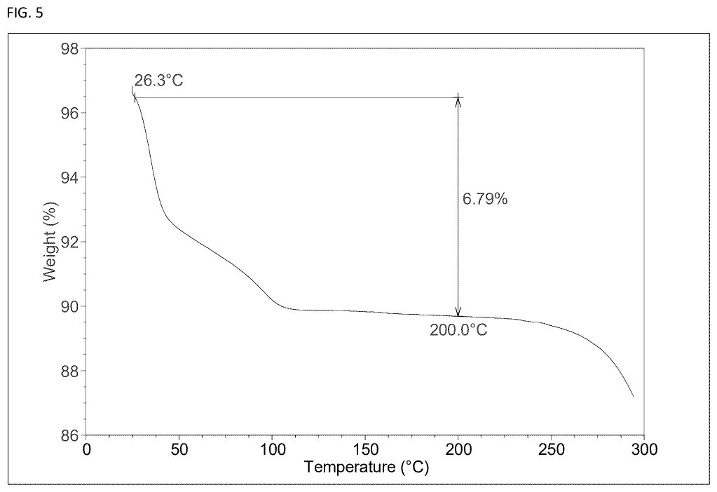

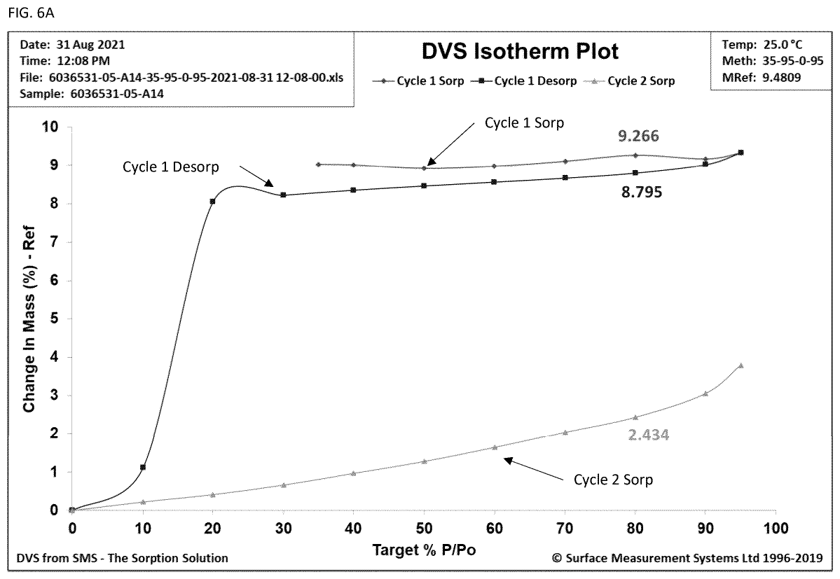

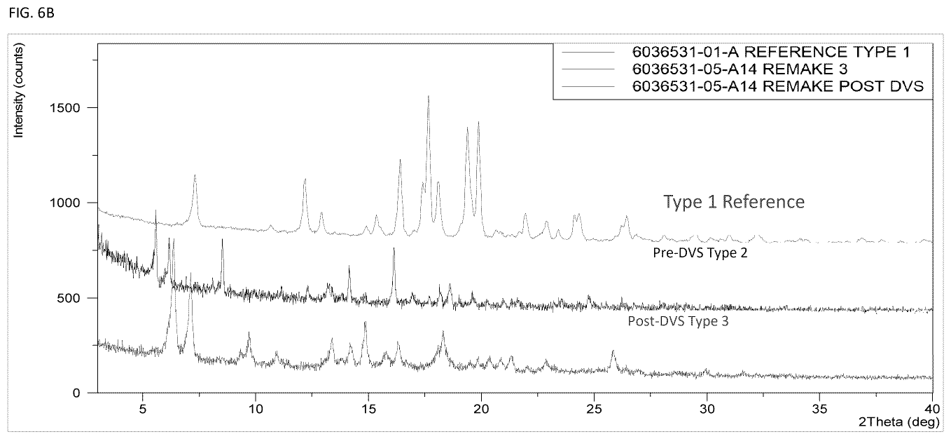

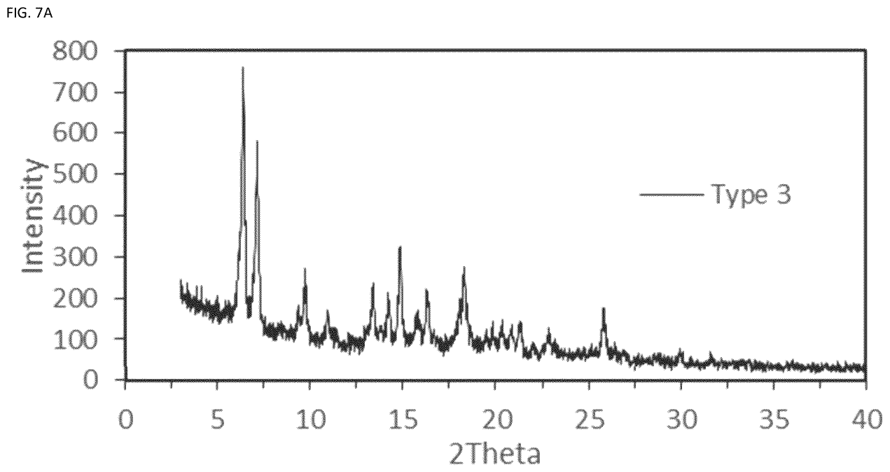

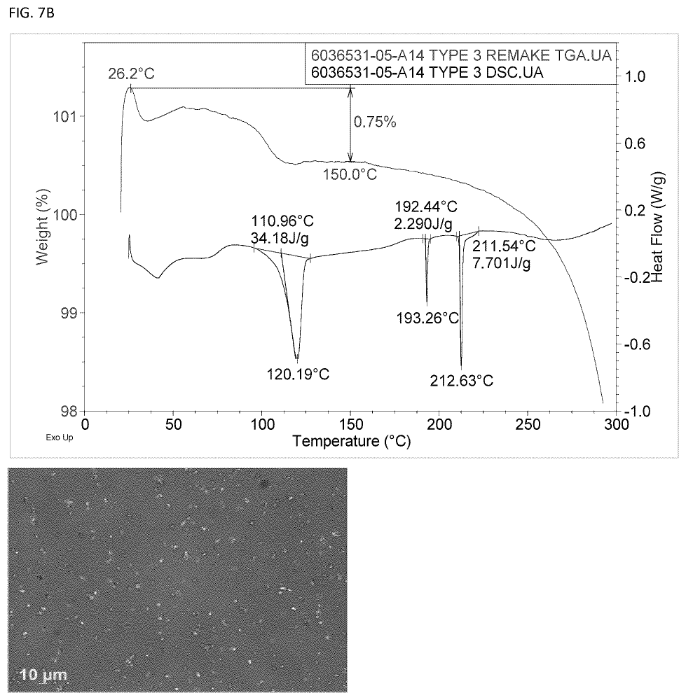

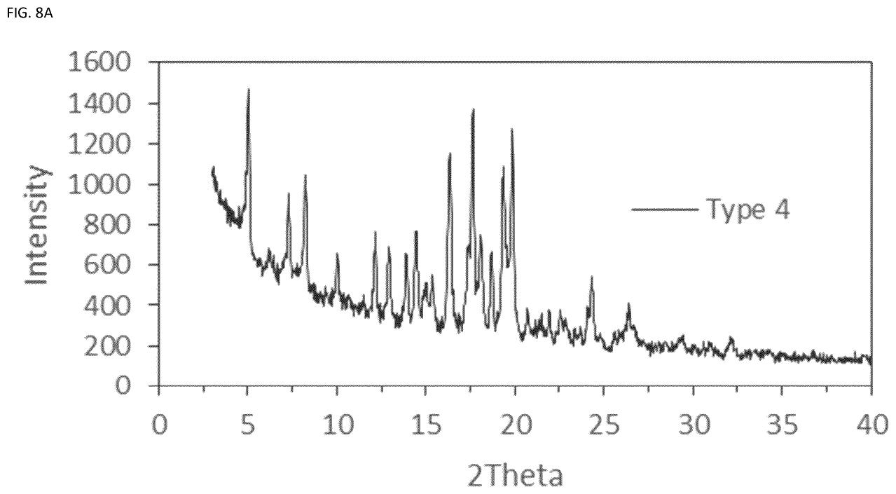

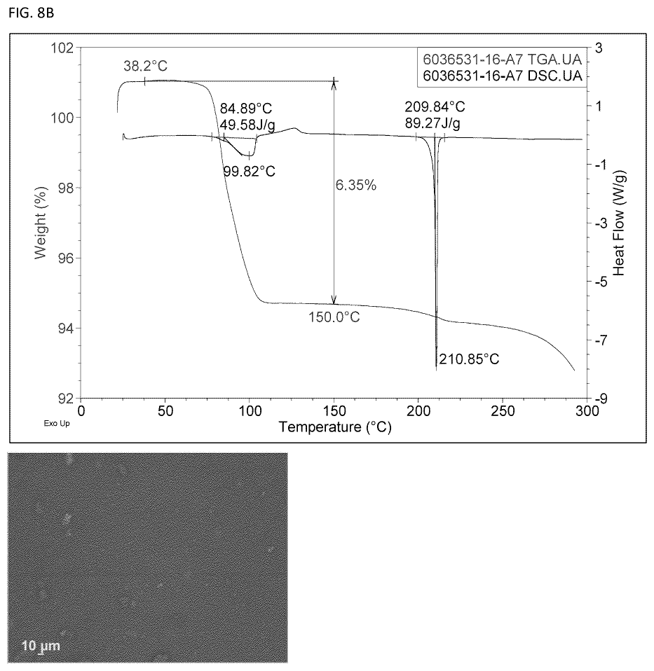

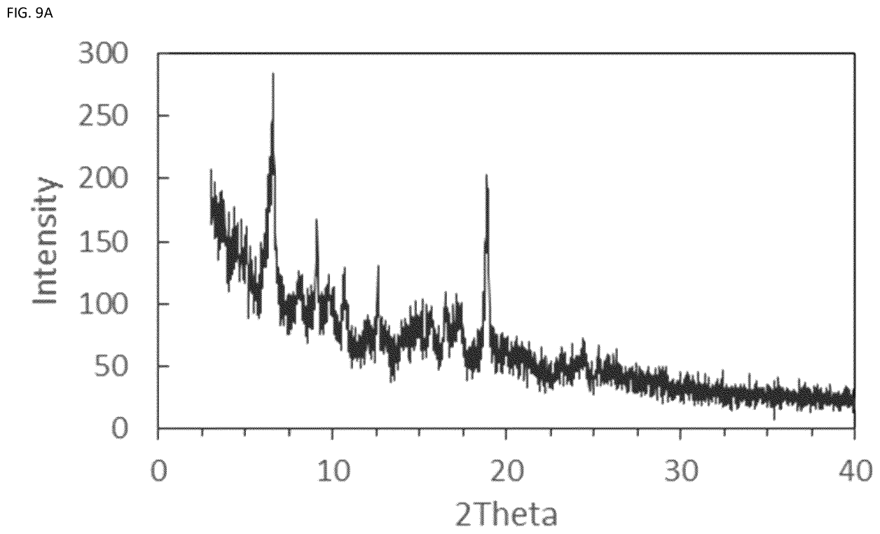

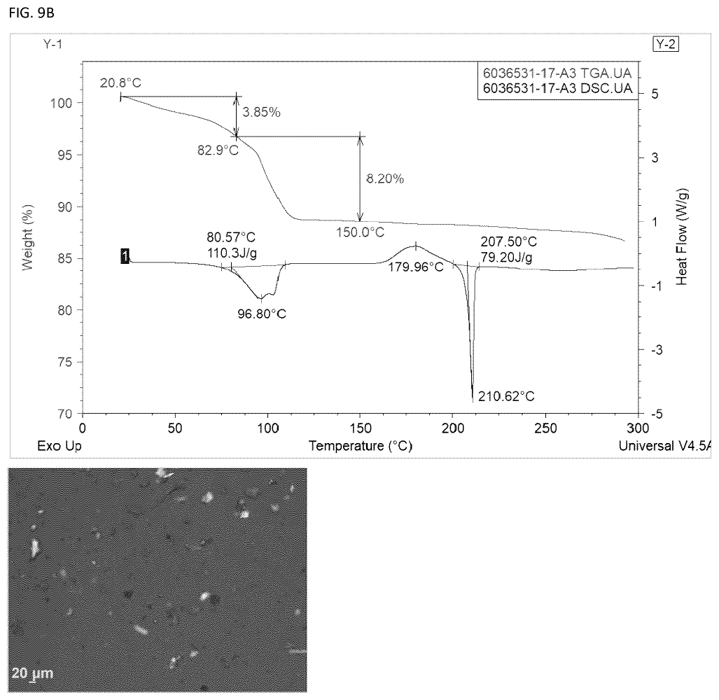

Table 15 provides a characterization summary of the crystalline forms of the present disclosure. FIGS. 1-9 illustrate the characterization of Type 1, Type 2, Type 3, Type 4, and Type 5, as indicated.

TABLE 15
Characterization Summary of Crystal Forms

| | | | DSC | |
|---|---|---|---|---|
| Crystal Form | Preparation | Weight Loss | (onset, | Form |
| (Sample ID) | Method | in TGA (%) | ° C.) | ID |
| Type 1 | Starting | 1.33% | 210.7 | N/A |
| 6036531-01-A | Material | KF 0.14% | | |
| Type 2 | Slurry in | 11.6 after air- | 78.1, | Tri- |
| 6036531-05- | THF:H₂O | drying for 1 | 93.6, | hydrate |
| A14 | (aw = 0.8) at | day 6.8% after | 211.7 | based on |
| | 5° C. | air-drying for | | DVS |
| | | 3 days; KF | | |
| | | 7.1% | | |
| Type 3 | Type 2 Post DVS | 0.75% | 111, | Mono- |
| 6036531-05- | | KF-3.0% | 192.4, | hydrate |
| A14 Post DVS | | | 211.5 | |
| Type 4 | Amorphous | 6.4% after air- | 84.9, | Sesqui- |
| 6036531-16-A7 | Slurry at RT | drying for 1 | 209.8 | hydrate |
| | using MTBE | day KF 4.5% | | |
| Type 5 | Amorphous Solid | 12.1% | 80.6, | Mono- |
| 6036531-17-A3 | Vapor Diffusion | KF 2.3% | 207.5 | DCM |
| | using DCM | | | solvate |

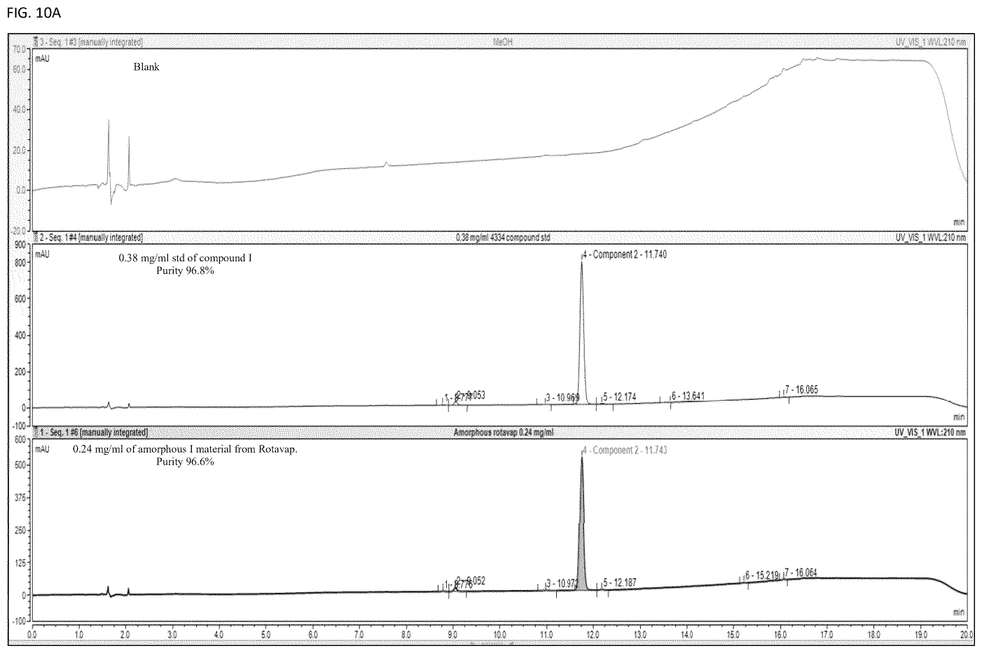

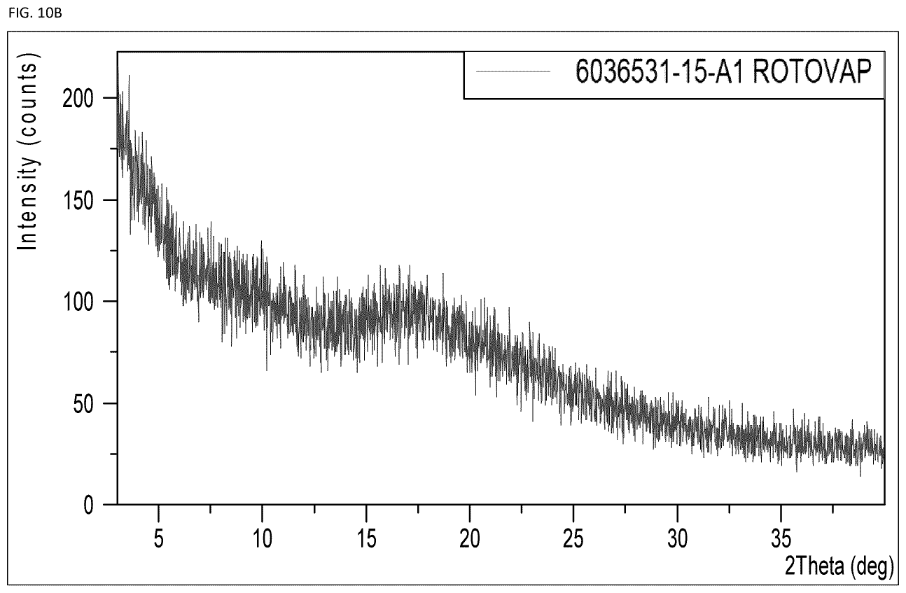

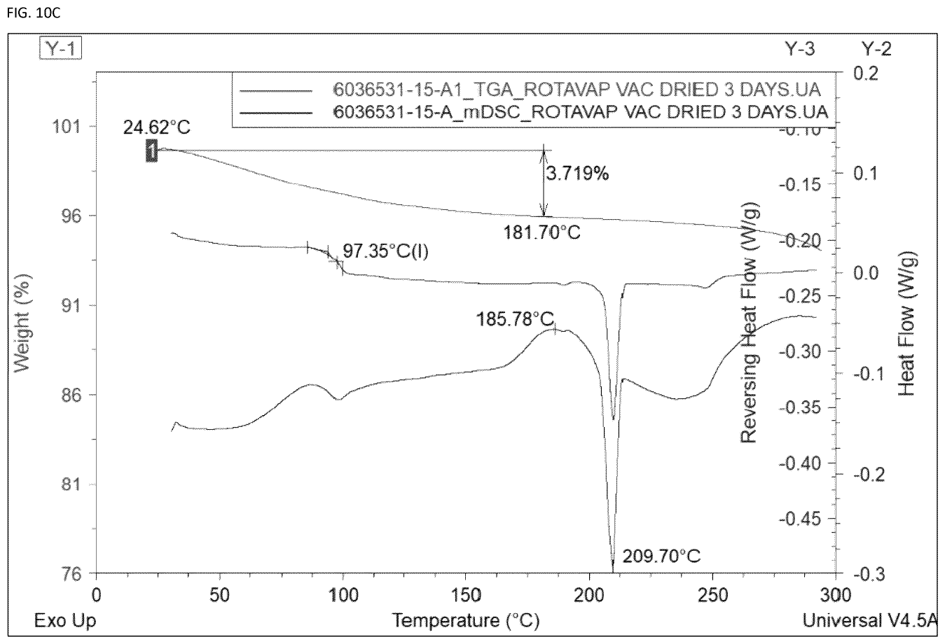

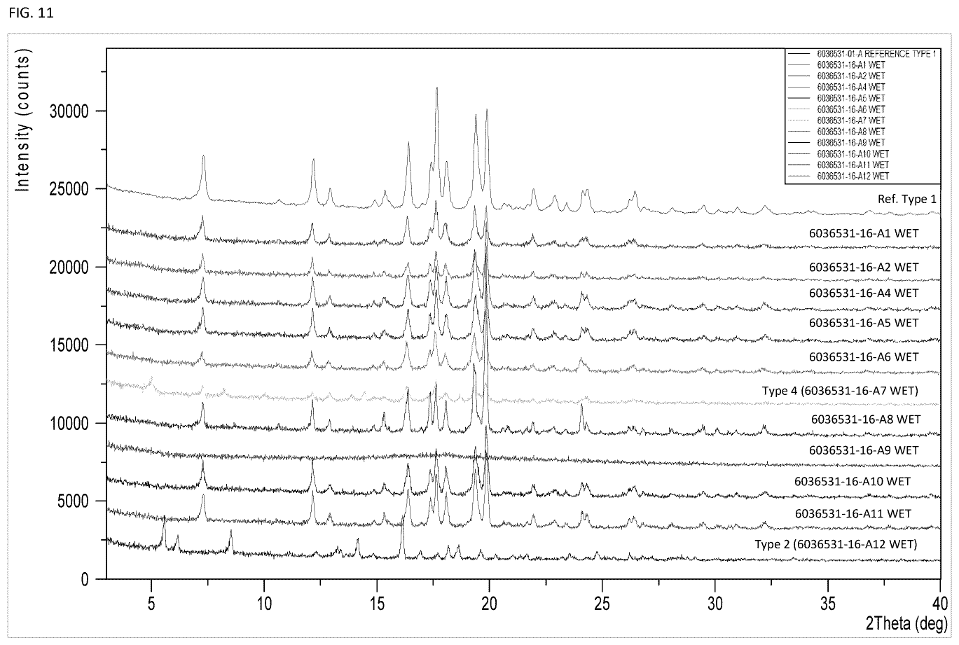

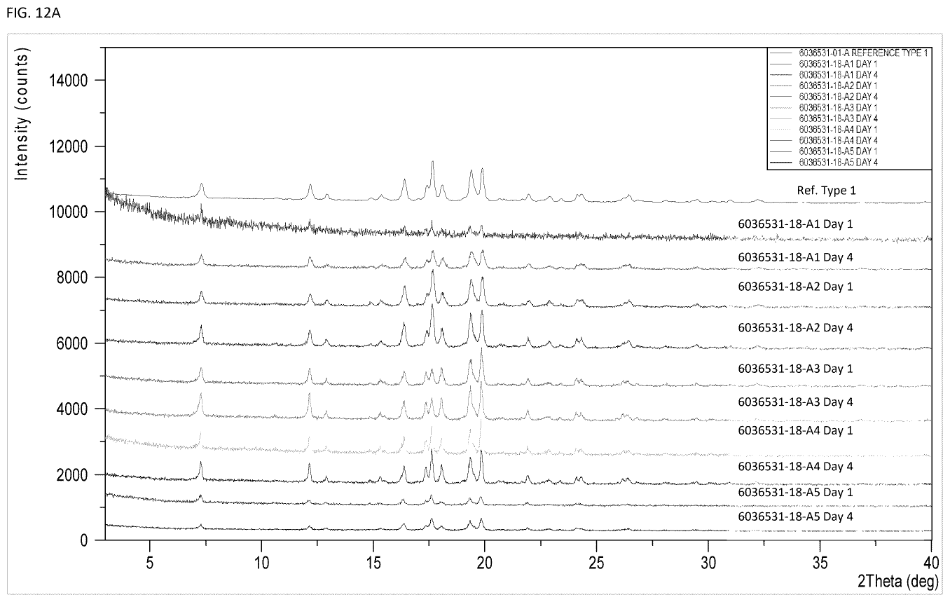

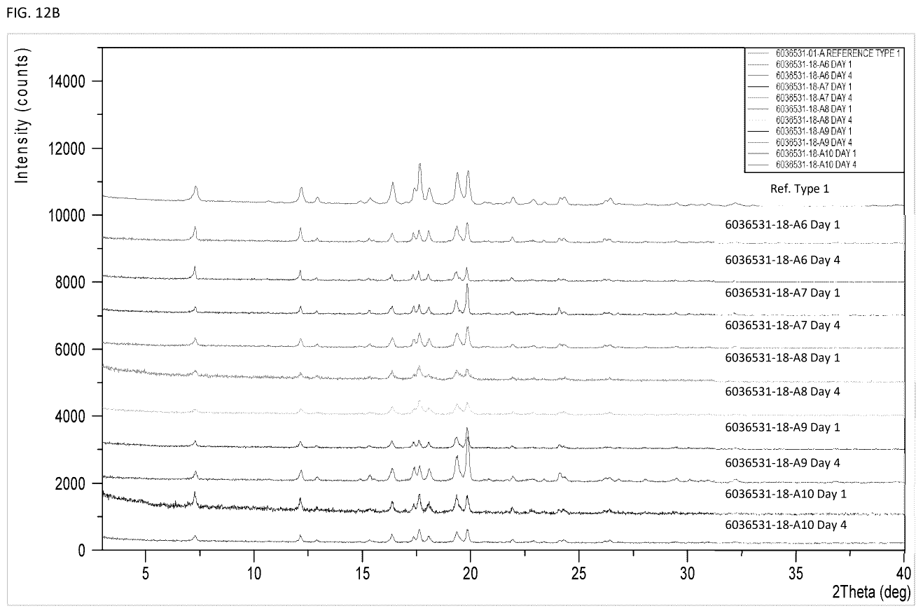

Table 16, Table 17, and Table 18 provide characterization summaries of the crystalline forms of the present disclosure following screening experiments using the amorphous form of Compound I as characterized in FIG. 10A, FIG. 10B, FIG. 10C. FIG. 11, FIG. 12A and FIG. 12B illustrate the characterization of Type 1, Type 2, Type 3, Type 4, and Type 5, as indicated.

TABLE 16
Slurry at RT starting with Amorphous form

| Experiment ID | Solvent | Solid Form |
|---|---|---|
| 6036531-16-A1 | H₂O | Type 1 |
| 6036531-16-A2 | THF | Type 1 |
| 6036531-16-A3 | DMF | Type 1 |
| 6036531-16-A4 | EtOAc | Type 1 |
| 6036531-16-A5 | EtOH | Type 1 |
| 6036531-16-A6 | Triethylamine | Type 1 |
| 6036531-16-A7 | MTBE | Type 4 |
| 6036531-16-A8 | Dioxane | Type 1 |
| 6036531-16-A9 | Heptane | Amorphous |
| 6036531-16-A10 | Dimethoxyethane | Type 1 |
| 6036531-16-A11 | Acetone | Type 1 |
| 6036531-16-A12 | THF/H₂O (aw = 0.8, 0.6/0.4) | Type 2 |

TABLE 17
Solid Vapor Diffusion starting with Amorphous form

| Experiment ID | Solvent | Solid Form |
|---|---|---|
| 6036531-17-A1 | THF | Type 1 |
| 6036531-17-A2 | MeOH | Type 1 |
| 6036531-17-A3 | DCM | Type 5 |
| 6036531-17-A4 | Acetone | Type 1 |
| 6036531-17-A5 | MTBE | Type 1 |
| 6036531-17-A6 | Dioxane | Type 1 |
| 6036531-17-A7 | Heptane | Amorphous |
| 6036531-17-A8 | Dimethoxyethane | Type 1 |
| 6036531-17-A9 | EtOAc | Type 1 |

TABLE 18
Temperature Cycling

| | | Solid Form | Solid Form |
|---|---|---|---|
| Experiment ID | Solvent | Day 1 | Day 4 |
| 6036531-18-A1 | THF | Type 1 | Type 1 |
| 6036531-18-A2 | H₂O | | |
| 6036531-18-A3 | MIBK | | |
| 6036531-18-A4 | EtOH | | |
| 6036531-18-A5 | Triethylamine | | |
| 6036531-18-A6 | MEK | | |
| 6036531-18-A7 | Dioxane | | |
| 6036531-18-A8 | Heptane | | |
| 6036531-18-A9 | Dimethoxyethane | | |
| 6036531-18-A10 | ACN | | |

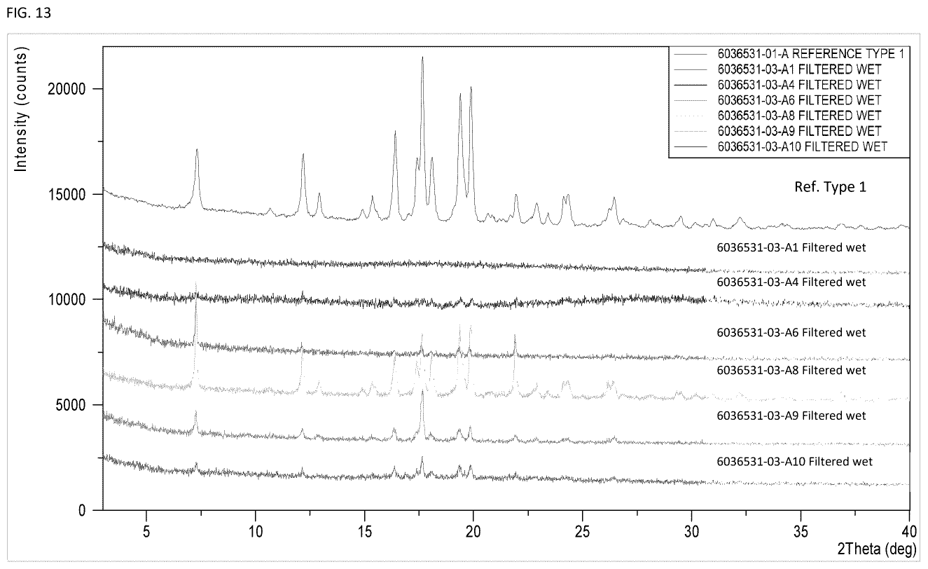

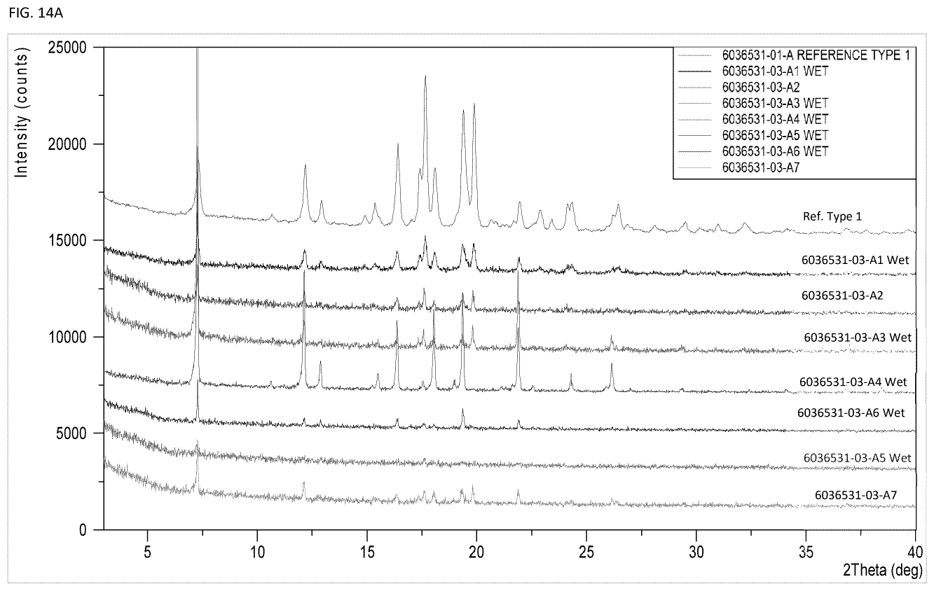

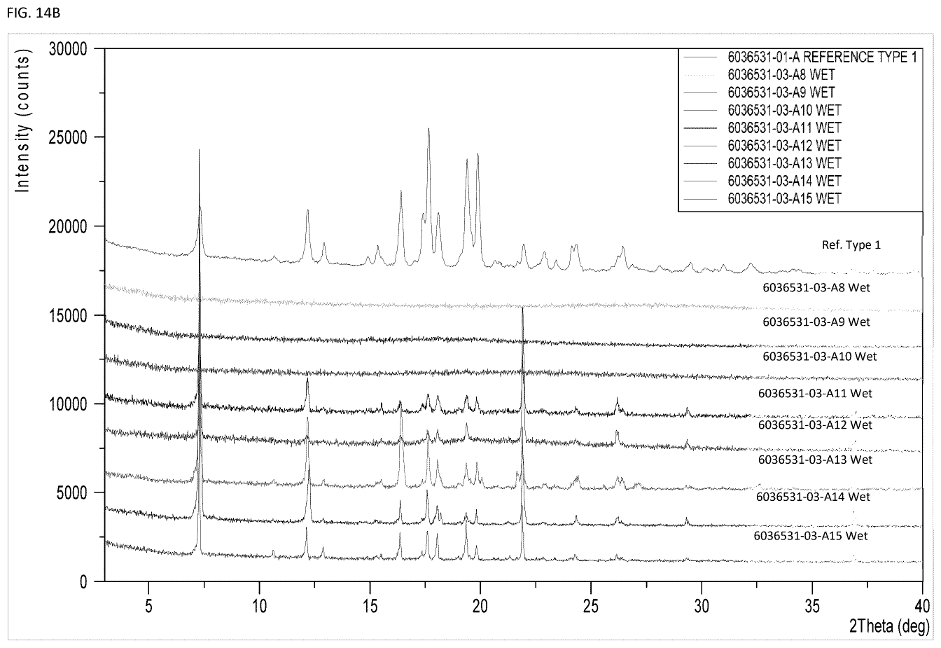

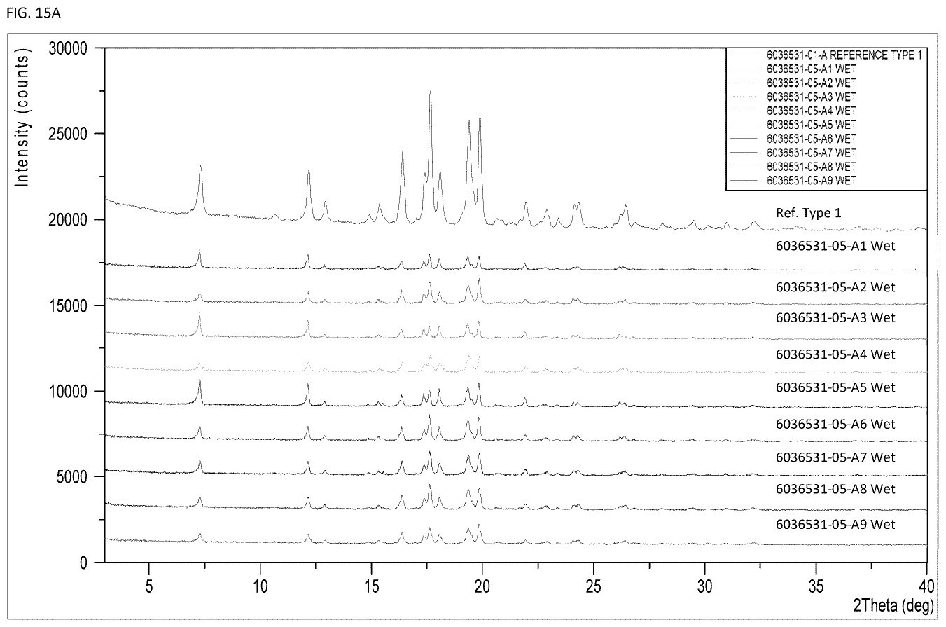

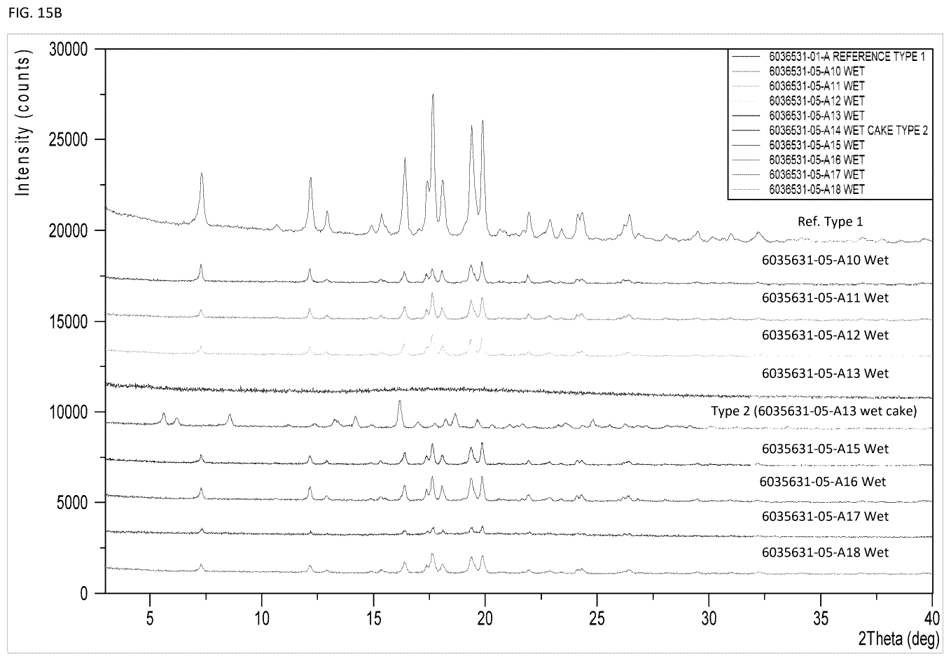

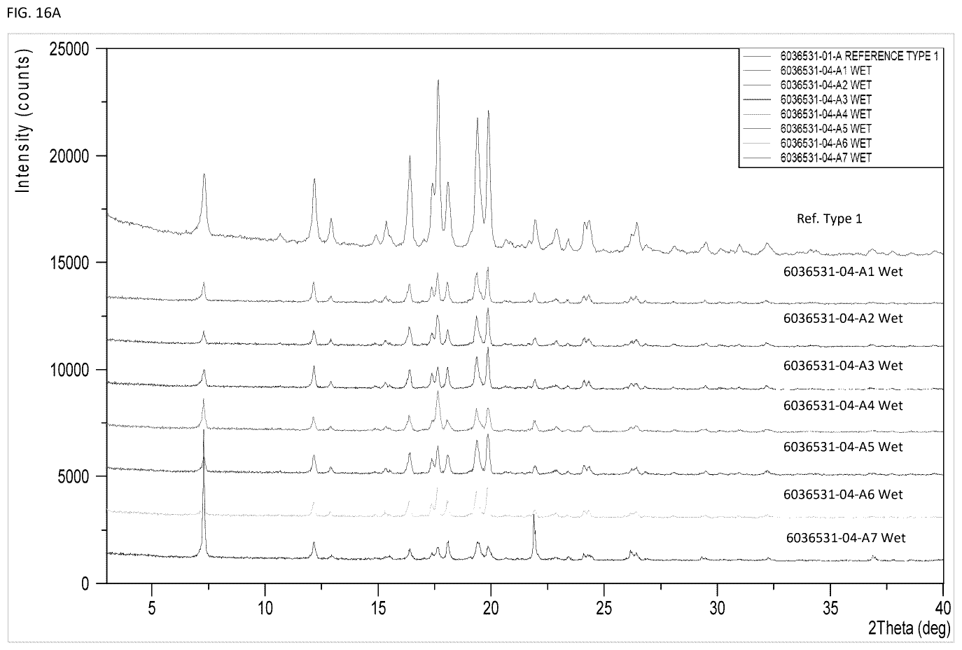

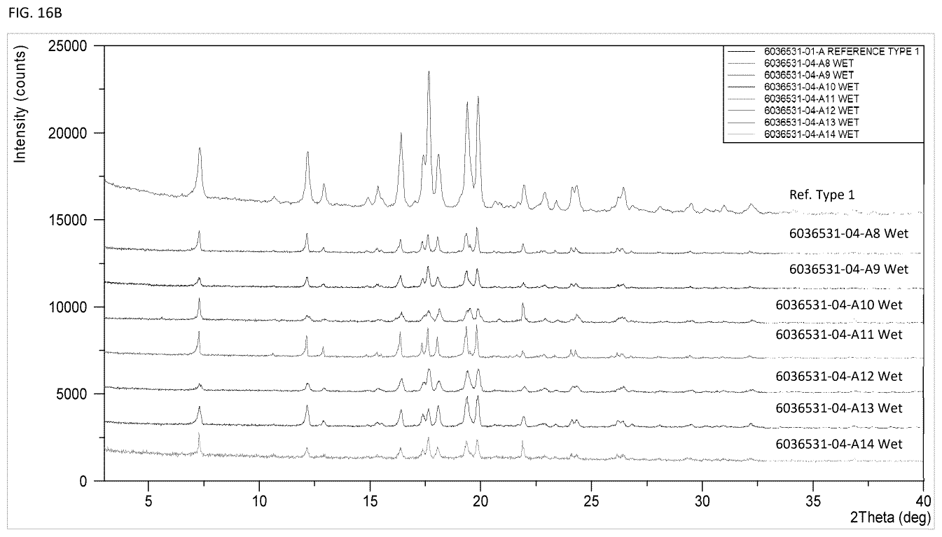

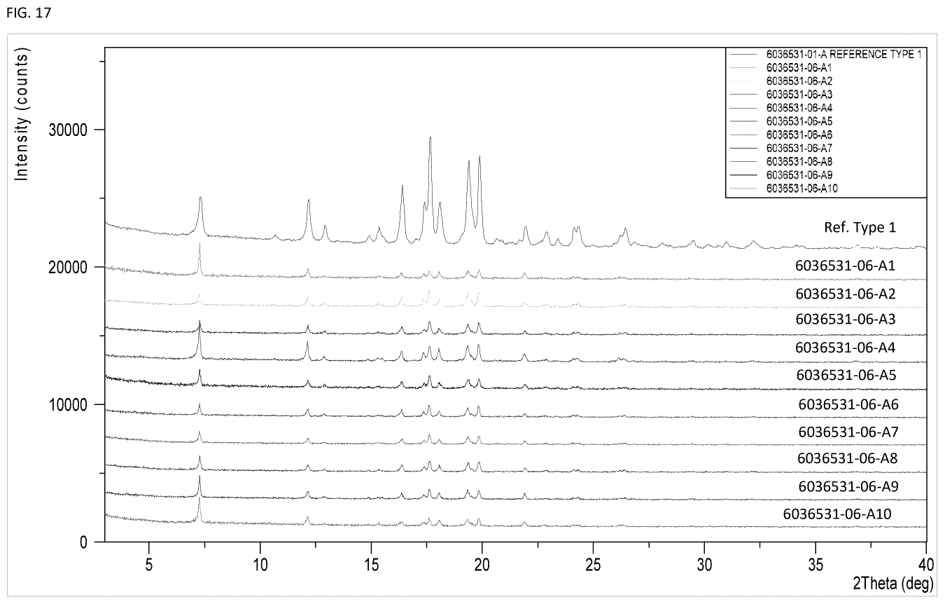

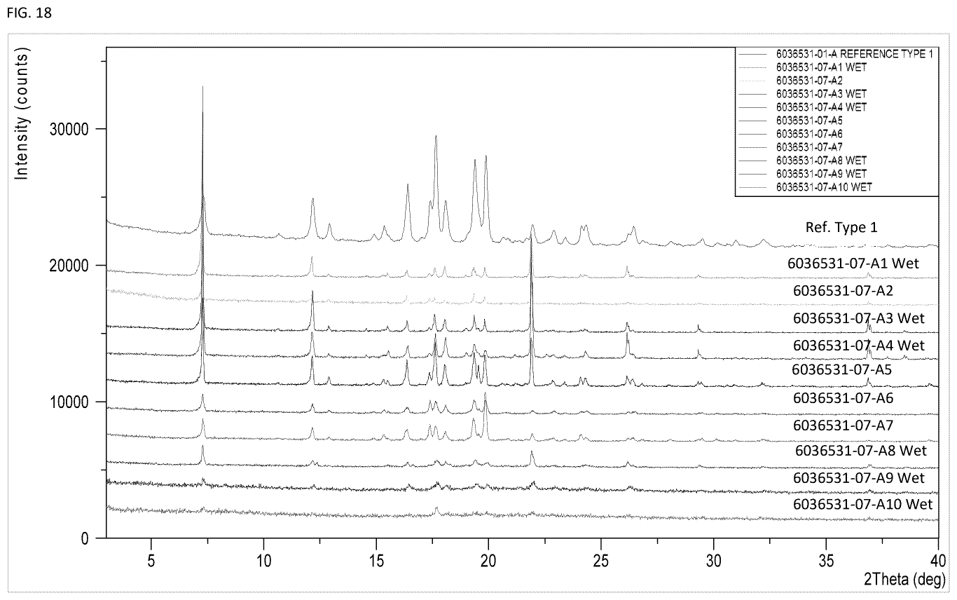

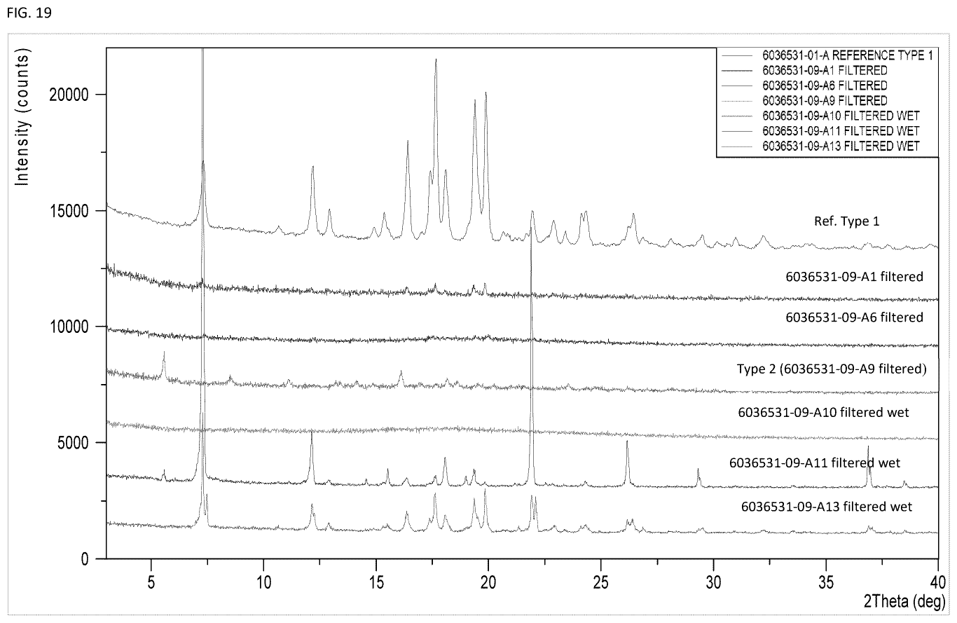

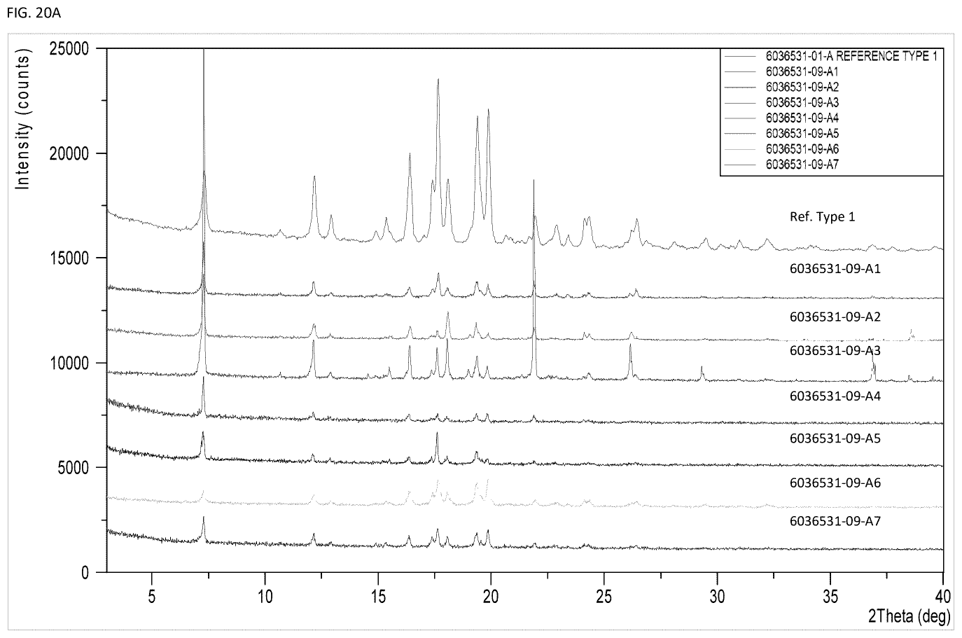

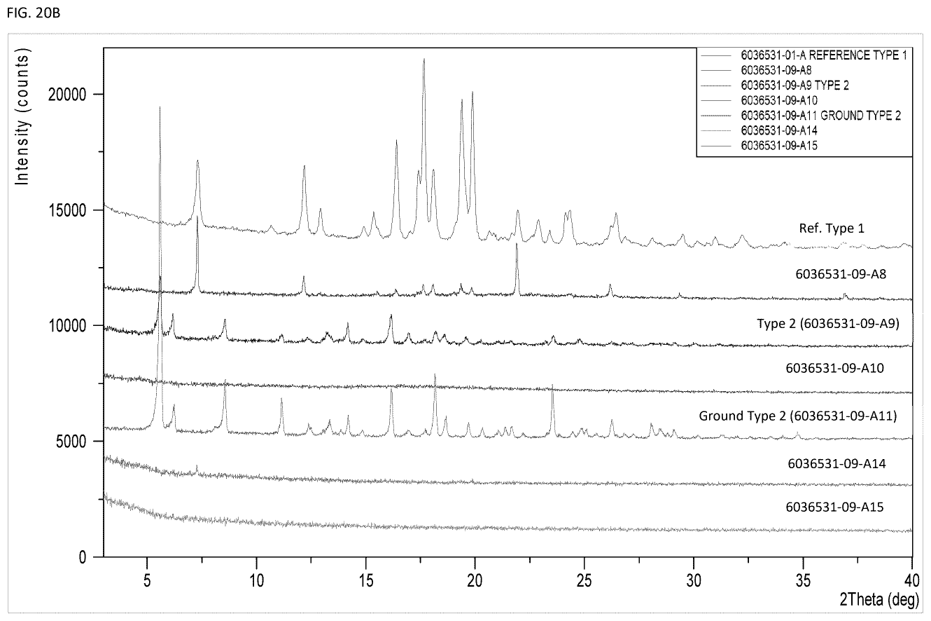

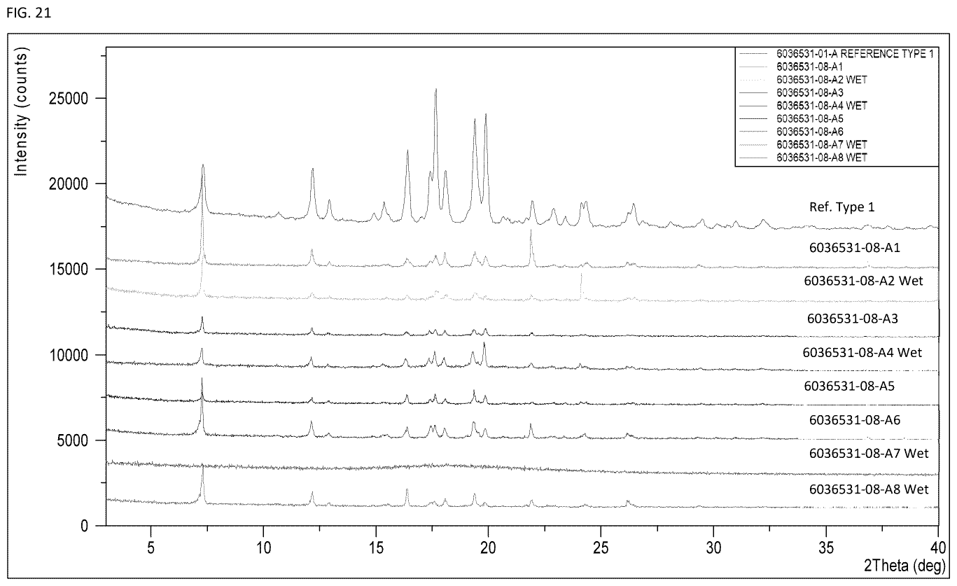

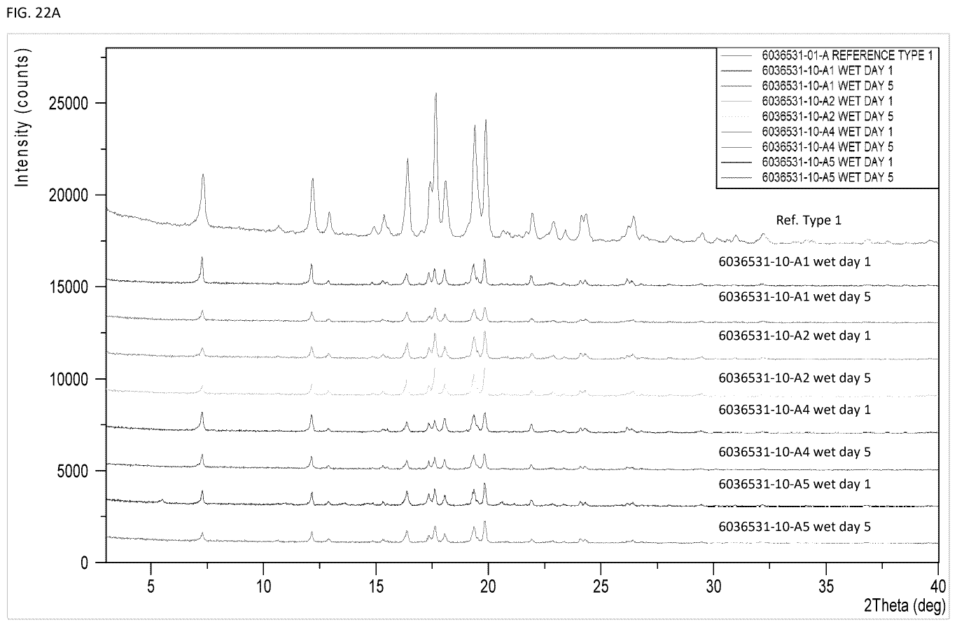

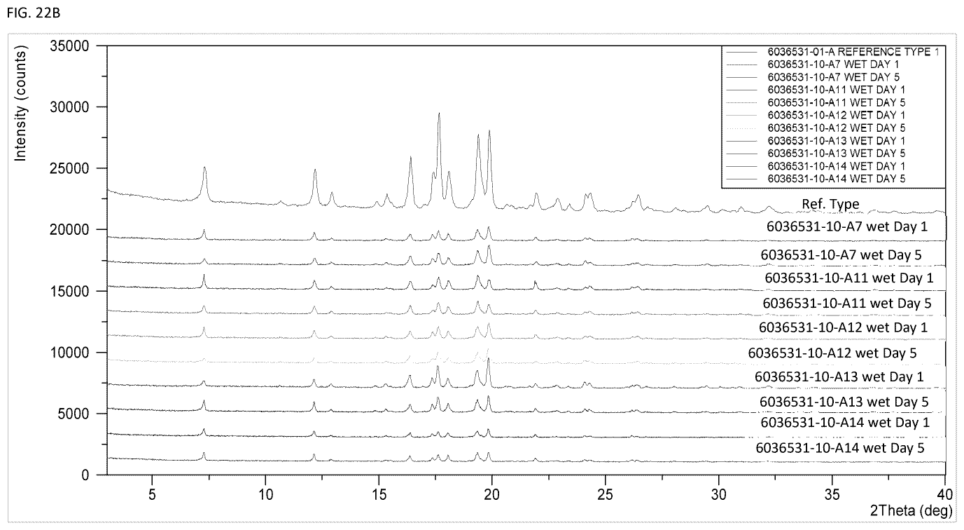

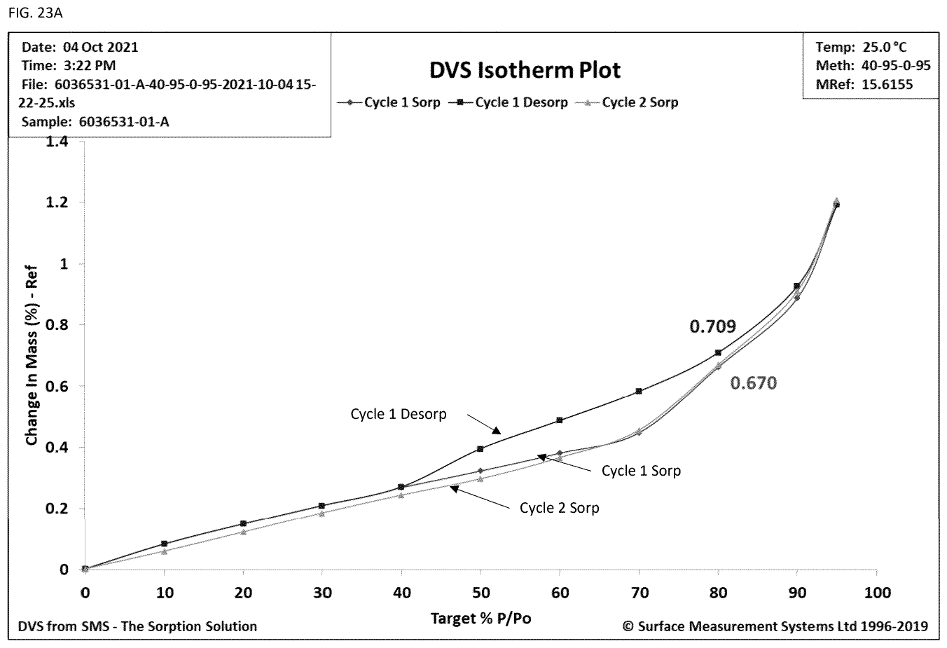

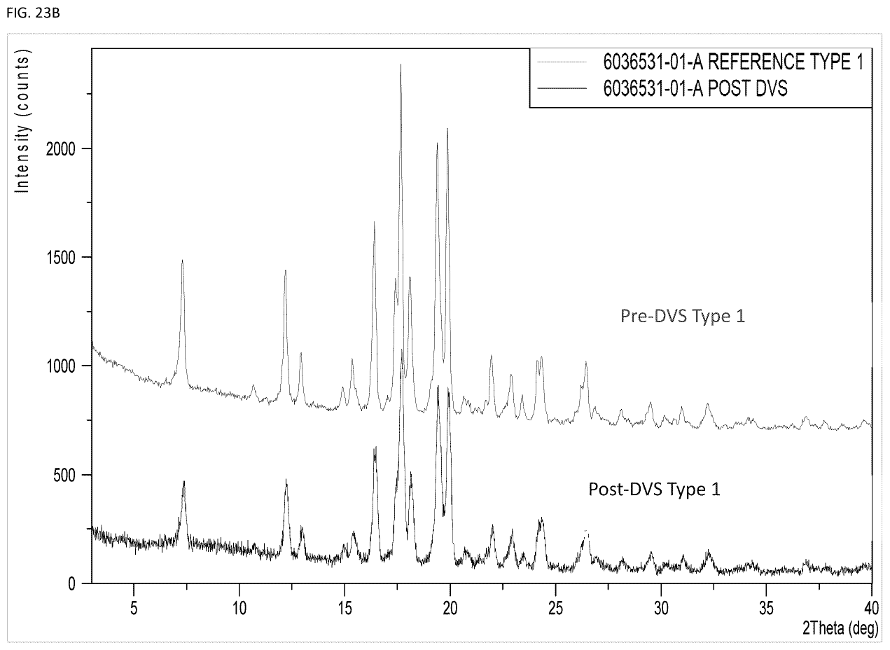

The following Table 19 to Table 28, each provide a characterization summary of the crystalline forms of the present disclosure following screening experiments using the crystalline Type 1 of Compound I. FIGS. 13-22 illustrate the characterization of Type 1, Type 2, Type 3, Type 4, and Type 5, as indicated.

TABLE 19
Filtered Anti Solvent Addition Experiments

| Experiment ID | Solvent | Anti Solvent | Solid Form |
|---|---|---|---|
| 6036531-03-A1 | MeOH | H₂O | Amorphous |
| 6036531-03-A2 | | ACN | Type 1 |
| 6036531-03-A3 | | MIBK | Type 4 |
| 6036531-03-A4 | Acetone | H₂O | Type 1 (low crystallinity) |
| 6036531-03-A5 | | MTBE | Amorphous |
| 6036531-03-A6 | | n-Heptane | Type 1 (low crystallinity) |
| 6036531-03-A7 | THF | DCM | Amorphous |
| 6036531-03-A8 | | H₂O | Type 1 |
| 6036531-03-A9 | | n-Heptane | Type 1 |
| 6036531-03-A10 | DMSO | H₂O | Type 1 |
| 6036531-03-A11 | | MTBE | Type 1 |
| 6036531-03-A12 | | ACN | No formation |
| 6036531-03-A13 | DMF | Toluene | No formation |
| 6036531-03-A14 | | DCM | No formation |
| 6036531-03-A15 | | ACN | Type 1 |

TABLE 20
Anti Solvent Addition

| Experiment ID | Solvent | Anti Solvent | Solid Form |
|---|---|---|---|
| 6036531-03-A1 | MeOH | H₂O | Type 1 |
| 6036531-03-A2 | | ACN | Type 1 |
| 6036531-03-A3 | | MIBK | Type 1 |
| 6036531-03-A4 | Acetone | H₂O | Type 1 |
| 6036531-03-A5 | | MTBE | Type 1 |
| 6036531-03-A6 | | n-Heptane | Amorphous |
| 6036531-03-A7 | THF | DCM | Type 1 |
| 6036531-03-A8 | | H₂O | Amorphous |
| 6036531-03-A9 | | h-Heptane | Amorphous |
| 6036531-03-A10 | DMSO | H₂O | Amorphous |
| 6036531-03-A11 | | MTBE | Type 1 |
| 6036531-03-A12 | | ACN | Type 1 |
| 6036531-03-A13 | DMF | Toluene | Type 1 |
| 6036531-03-A14 | | DCM | Type 1 |
| 6036531-03-A15 | | ACN | Type 1 |

TABLE 21
Slurry at RT

| Experiment ID | Solvent | Solid Form |
|---|---|---|
| 6036531-05-A1 | water | Type 1 |
| 6036531-05-A2 | ACN | Type 1 |
| 6036531-05-A3 | MIBK | Type 1 |
| 6036531-05-A4 | IPAc | Type 1 |
| 6036531-05-A5 | IPA | Type 1 |
| 6036531-05-A6 | CPME | Type 1 |
| 6036531-05-A7 | DCM | Type 1 |
| 6036531-05-A8 | Toluene | Type 1 |
| 6036531-05-A9 | Acetone | Type 1 |
| 6036531-05-A10 | MEK | Type 1 |
| 6036531-05-A11 | MeOH | Type 1 |
| 6036531-05-A12 | n-Heptane | Type 1 |
| 6036531-05-A13 | THF/H₂O (aw = 0.6, 0.73/0.27) | Amorphous |
| 6036531-05-A14 | THF/H₂O (aw = 0.8, 0.6/0.4) | Type 2 |
| 6036531-05-A15 | Acetone/H₂O (aw = 0.2, 0.941/0.059) | Type 1 |
| 6036531-05-A16 | Acetone/H₂O (aw = 0.8, 0.492/0.508) | Type 1 |
| 6036531-05-A17 | DMSO/MTBE (1:2) | Type 1 |
| 6036531-05-A18 | Acetone/n-Heptane (1:2) | Type 1 |

TABLE 22
Slurry at 60° C.

| Experiment ID | Solvent | Solid Form |
|---|---|---|
| 6036531-04-A1 | water | Type 1 |
| 6036531-04-A2 | ACN | Type 1 |
| 6036531-04-A3 | MIBK | Type 1 |
| 6036531-04-A4 | IPAc | Type 1 |
| 6036531-04-A5 | MEK | Type 1 |
| 6036531-04-A6 | Toluene | Type 1 |
| 6036531-04-A7 | EtOH | Type 1 |
| 6036531-04-A8 | n-Propanol | Type 1 |
| 6036531-04-A9 | n-Heptane | Type 1 |
| 6036531-04-A10 | THF/H₂O (1:2) | Type 1 |
| 6036531-04-A11 | DMF/Toluene (1:2) | Type 1 |
| 6036531-04-A12 | MEK/n-Heptane (1:1) | Type 1 |
| 6036531-04-A13 | 1-Butanol/MIBK (1:1) | Type 1 |
| 6036531-04-A14 | DMSO/H₂O (1:2) | Type 1 |

TABLE 23
Solid Vapor Diffusion

| Experiment ID | Solvent | Solid Form |
|---|---|---|
| 6036531-06-A1 | THF | Type 1 |
| 6036531-06-A2 | Acetone | Type 1 |
| 6036531-06-A3 | n-Heptane | Type 1 |
| 6036531-06-A4 | MeOH | Type 1 |
| 6036531-06-A5 | ACN | Type 1 |
| 6036531-06-A6 | DCM | Type 1 |
| 6036531-06-A7 | MTBE | Type 1 |
| 6036531-06-A8 | IPAc | Type 1 |
| 6036531-06-A9 | MEK | Type 1 |
| 6036531-06-A10 | IPA | Type 1 |

TABLE 24
Liquid Vapor Diffusion

| Experiment ID | Solvent | Anti Solvent | Solid Form |
|---|---|---|---|
| 6036531-07-A1 | EtOH | DCM | Type 1 |
| 6036531-07-A2 | | IPAc | Type 1 |
| 6036531-07-A3 | DMF | DCM | Type 1 |
| 6036531-07-A4 | | MTBE | Type 1 |
| 6036531-07-A5 | THF | ACN | Type 1 |
| 6036531-07-A6 | | n-Heptane | Type 1 |
| 6036531-07-A7 | | MTBE | Type 1 |
| 6036531-07-A8 | DMSO | IPAc | Type 1 |
| 6036531-07-A9 | | ACN | Type 1 |
| 6036531-07-A10 | | n-Heptane | Type 1 (low crystallinity) |

TABLE 25
Filtered Slow Cooling Experiments

| Experiment ID | Solvent | Solid Form |
|---|---|---|
| 6036531-09-A1 | MeOH | Type 1 (low crystallinity) |
| 6036531-09-A3 | DMF | No formation |
| 6036531-09-A6 | THF | Amorphous |
| 6036531-09-A8 | DMSO | Type 1 |
| 6036531-09-A9 | THF/H₂O | Type 2 |
| | (aw = 0.2, 0.92/0.08) | |
| 6036531-09-A10 | THF/H₂O | Amorphous |
| | (aw = 0.8, 0.6/0.4) | |
| 6036531-09-A11 | Acetone/H₂O (1:1) | Type 1 |
| 6036531-09-A13 | 1-Butanol/Toluene (1:1) | Type 1 |

TABLE 26
Slow Evaporation

| Experiment ID | Solvent | Solid Form |
|---|---|---|
| 6036531-08-A1 | MeOH | Type 1 |
| 6036531-08-A2 | Acetone | Type 1 |
| 6036531-08-A3 | 2-MeTHF | Type 1 |
| 6036531-08-A4 | THF | Type 1 |
| 6036531-08-A5 | MEK | Type 1 |
| 6036531-08-A6 | Ethanol/ACN (1:1) | Type 1 |
| 6036531-08-A7 | THF/DCM (1:2) | Amorphous |
| 6036531-08-A8 | Acetone/DCM (1:1) | Type 1 |

TABLE 27
Slow Cooling

| Experiment ID | Solvent | Solid Form |
|---|---|---|
| 6036531-09-A1 | MeOH | Type 1 |
| 6036531-09-A2 | Acetone | Type 1 |
| 6036531-09-A3 | DMF | Type 1 |
| 6036531-09-A4 | IPA | Type 1 |
| 6036531-09-A5 | MEK | Type 1 |
| 6036531-09-A6 | THF | Type 1 |
| 6036531-09-A7 | 2-MeTHF | Type 1 |
| 6036531-09-A8 | DMSO | Type 1 |
| 6036531-09-A9 | THF/H₂O (aw = 0.2, 0.92/0.08) | Type 2 |
| 6036531-09-A10 | THF/H₂O (aw = 0.8, 0.6/0.4) | Amorphous |
| 6036531-09-A11 | Acetone/H₂O (1:1) | Type 2 |
| 6036531-09-A12 | DMSO/H₂O (1:2) | No formation |
| 6036531-09-A13 | 1-Butanol/Toluene (1:1) | Amorphous - |
| 6036531-09-A14 | MEK/n-Heptane (1:1) | Amorphous |
| 6036531-09-A15 | THF/n-Heptane (1:2) | Amorphous |

TABLE 28
Temperature Cycling

| | | Solid Form | Solid Form |
|---|---|---|---|
| Experiment ID | Solvent | (Day 1) | (Day 5) |
| 6036531-10-A1 | IPA | Type 1 | Type 1 |
| 6036531-10-A2 | ACN | Type 1 | Type 1 |
| 6036531-10-A3 | DMF | No formation | No formation |
| 6036531-10-A4 | MIBK | Type 1 | Type 1 |
| 6036531-10-A5 | MEK | Type 1 | Type 1 |
| 6036531-10-A6 | THF | No formation | No formation |
| 6036531-10-A7 | 2-MeTHF | Type 1 | Type 1 |
| 6036531-10-A8 | DMSO | No formation | No formation |
| 6036531-10-A9 | THF/H₂O (aw = | No formation | No formation |
| | 0.2, 0.92/0.08) | | |
| 6036531-10-A10 | THF/H₂O (aw = | No formation | No formation |
| | 0.8, 0.6/0.4) | | |
| 6036531-10-A11 | EtOH/H₂O (1:1) | Type 1 | Type 1 |
| 6036531-10-A12 | n-propanol/ | Type 1 | Type 1 |
| | n-Heptane (1:1) | | |
| 6036531-10-A13 | DMF/ACN (1:2) | Type 1 | Type 1 |
| 6036531-10-A14 | IPA/MIBK (1:1) | Type 1 | Type 1 |

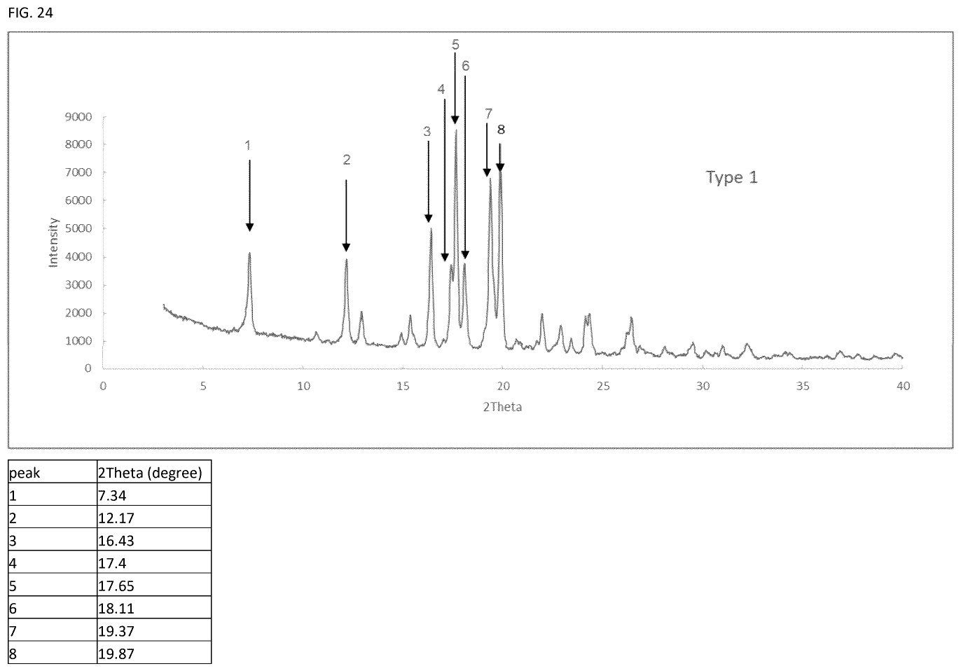

FIG. 24 is an XRPD of Compound I, Type 1. Particular characterization peaks present in the XRPD diffractogram for Type 1 are presented in Table 29 in 20. Peak positions present in the XRPD diffractogram acquired for Type 1 are presented in Table 30.

TABLE 29
Characteristic Peaks - Type 1

| peak | 2Theta (degree) |
|---|---|
| 1 | 7.34 |
| 2 | 12.17 |
| 3 | 16.43 |
| 4 | 17.4 |
| 5 | 17.65 |
| 6 | 18.11 |
| 7 | 19.37 |
| 8 | 19.87 |

TABLE 30
XRPD peak positions for Type 1
Peak position in °2θ

4.2
5.0
5.8
6.1
7.3
10.7
11.2
12.2
12.9
13.5
14.0
14.9
15.4
16.4
17.0
17.4
17.7
18.1
19.4
19.9
20.7
20.9
21.4
21.7
21.9
22.9
23.4
24.1
24.3
25.0
26.2
26.5
26.8
27.5
28.1
28.5
29.5
30.2
31.0
32.2
33.1
33.5
34.2
34.4
34.9
35.6
36.2
36.9
37.3
37.8

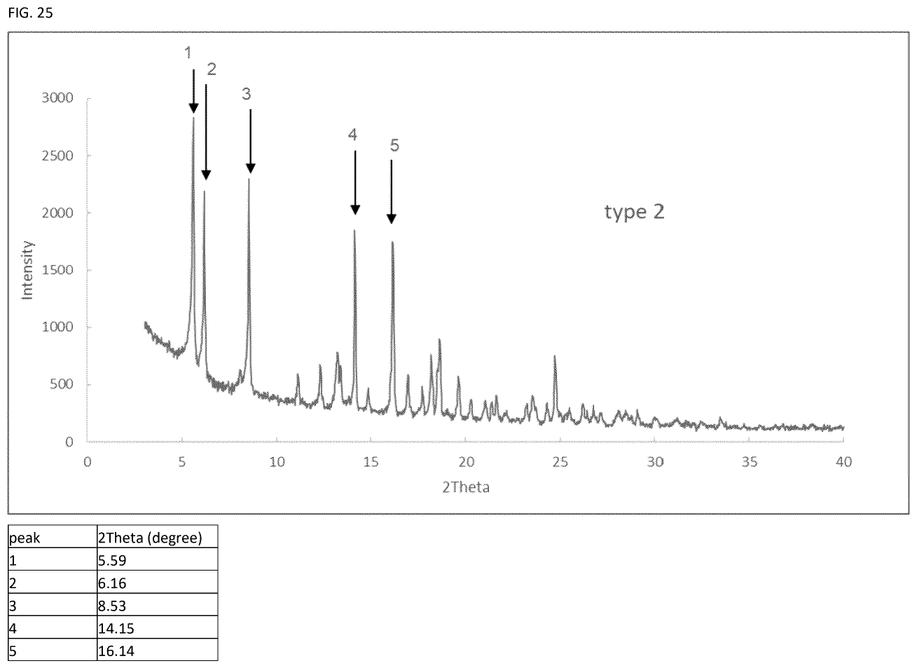

FIG. 25 is an XRPD of Compound I, Type 2. Particular characterization peaks present in the XRPD diffractogram for Type 2 are presented in Table 31 in 20. Peak positions present in the XRPD diffractogram acquired for Type 2 are presented in Table 32.

TABLE 31
Characteristic Peaks - Type 2

| peak | 2Theta (degree) |
|---|---|
| 1 | 5.59 |
| 2 | 6.16 |
| 3 | 8.53 |
| 4 | 14.15 |
| 5 | 16.14 |

TABLE 32
XRPD peaks positions for Type 2
Peak position in °2θ

4.3
5.6
6.2
8.1
8.5
9.9
11.1
12.3
13.2
14.2
14.9
16.1
16.9
17.7
18.2
18.6
19.6
20.3
21.0
21.4
21.6
22.1
23.2
23.5
24.3
24.7
25.5
26.2
26.8
27.2
28.1
29.1
30.0
31.2
33.5
34.7
38.3

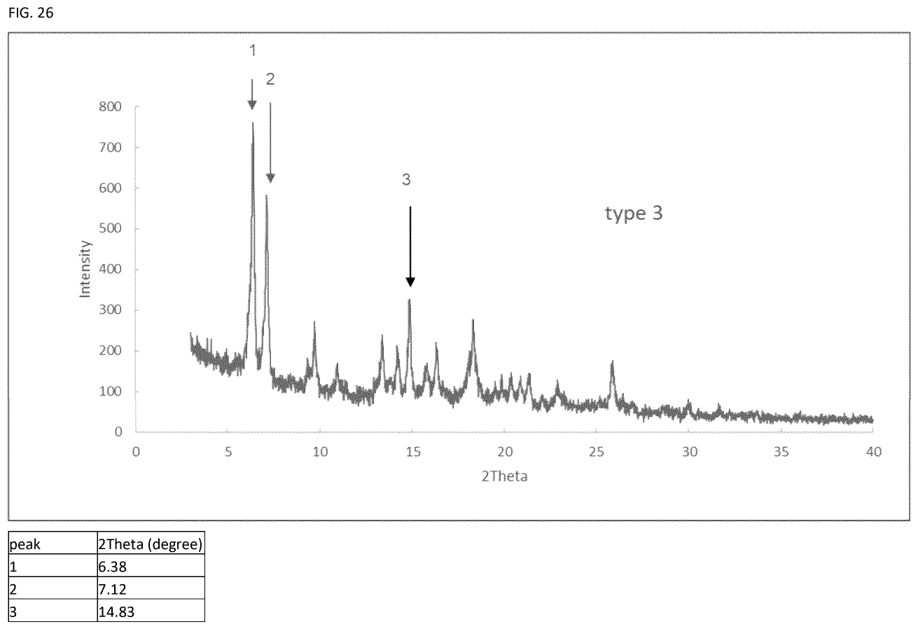

FIG. 26 is an XRPD of Compound I, Type 3. Particular characterization peaks in the XRPD diffractogram for Type 3 are presented in Table 33 in 20. Peak positions present in the XRPD diffractogram acquired for Type 3 are presented in Table 34.

TABLE 33
Characteristic Peaks - Type 3

| peak | 2Theta (degree) |
|---|---|
| 1 | 6.38 |
| 2 | 7.12 |
| 3 | 14.83 |

TABLE 34
XRPD peaks positions for Type 3
Peak position in °2θ

6.4
7.1
8.5
9.3
9.7
10.9
13.4
14.2
14.9
15.8
16.3
18.3
20.4
20.9
21.4
22.0
22.9
25.8
28.7
29.9
31.6
33.7

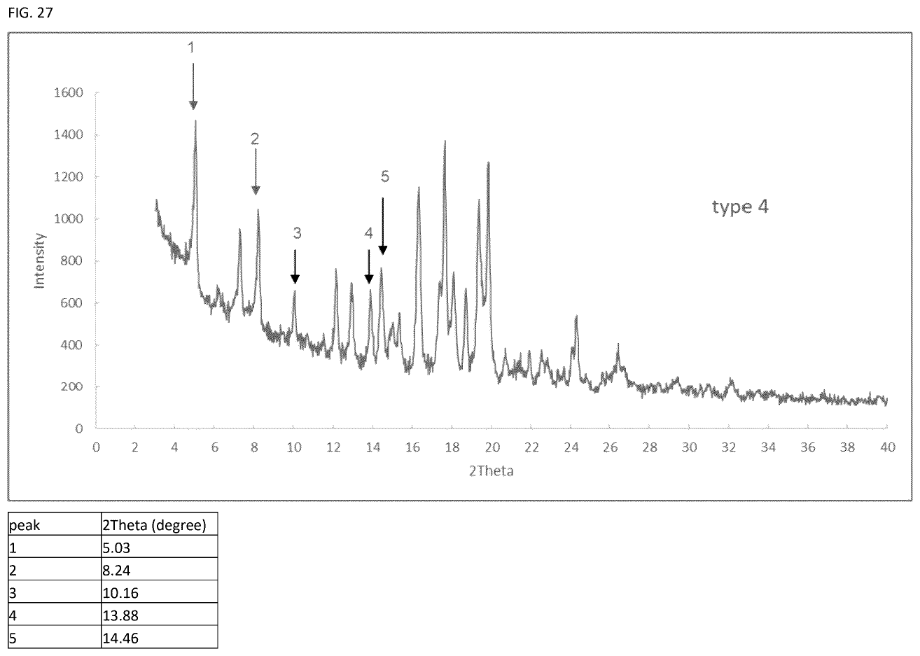

FIG. 27 is an XRPD of Compound I, Type 4. Particular characterization peaks in the XRPD diffractogram for Type 4 are presented in Table 35 in 20. Peak positions present in the XRPD diffractogram acquired for Type 4 are presented in Table 36.

TABLE 35
Characteristic Peaks - Type 4

| peak | 2Theta (degree) |
|---|---|
| 1 | 5.03 |
| 2 | 8.24 |
| 3 | 10.16 |
| 4 | 13.88 |
| 5 | 14.46 |

TABLE 36
XRPD peak positions for Type 4
Peak position in °2θ

5.1
6.2
7.3
8.3
9.4
10.0
11.5
12.2
13.0
13.9
14.5
15.0
15.4
16.4
17.3
17.6
18.1
18.7
19.3
19.9
20.7
21.9
22.5
23.4
24.3
25.7
26.5
28.5
29.5
30.6
31.0
32.2
34.3
39.6

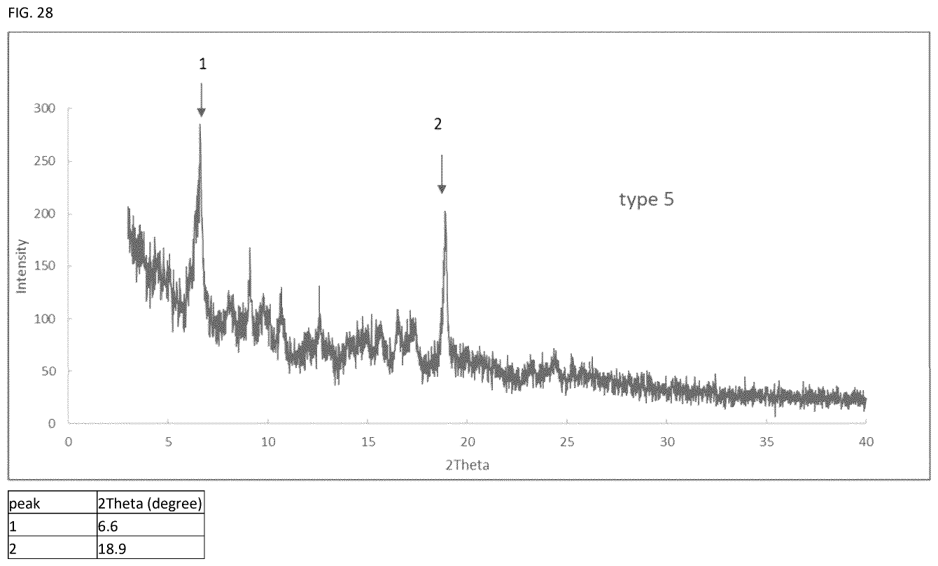

FIG. 28 is an XRPD of Compound I, Type 5. Particular characterization peaks in the XRPD diffractogram for Type 5 are presented in Table 37 in 20. Peak positions present in the XRPD diffractogram acquired for Type 5 are presented in Table 38.

TABLE 37
Characteristic Peaks - Type 5

| peak | 2Theta (degree) |
|---|---|
| 1 | 6.6 |
| 2 | 18.9 |

TABLE 38
XRPD peak positions for Type 5
Peak position in °2θ

5.1
5.7
6.7
8.0
9.1
9.8
10.1
10.7
11.9
12.6
14.0
14.9
15.6
16.5
17.3
18.9
20.6
21.4
23.3
24.3
26.3
27.6
27.9
28.7
30.2

### Solubility Experiments

The solubility of Compound I, Type 1 (6036531-01-A) was estimated at RT in twenty different solvents. Approximately 2 mg solids were added into a 3-mL glass vial. Solvents in Table 39 were then added stepwise (50 μL per step) into the vials until the solids were dissolved or a total volume of 2 mL was reached. The results are summarized in Table 39.

TABLE 39
Approximate Solubility Compound I (6036531-01-A)

| | Approximate Solubility | |
|---|---|---|
| Solvent | (mg/mL) at RT | Characterization |
| H₂O | S < 2.5 | Poor |
| MeOH | S > 42.0 | High |
| EtOH | 20.0 < S < 40.0 | High |
| IPA | 2.2 < S < 7.3 | Moderate |
| Acetonitrile | S < 2.0 | Poor |
| Acetone | 2.3 < S < 7.7 | Moderate |
| MIBK | S < 2.2 | Poor |
| IPAc | S < 2.1 | Poor |
| THF | 21.0 < S < 42.0 | High |
| MTBE | S < 2.0 | Poor |
| CPME | S < 2.2 | Poor |
| DCM | S < 2.4 | Poor |
| DMSO | S > 48.0 | High |
| DMF | S > 48.0 | High |
| Toluene | S < 2.5 | Poor |
| n-Heptane | S < 2.3 | Poor |
| 1-Butanol | 2.4 < S < 8.0 | Moderate |
| n-Propanol | 2.2 < S < 7.3 | Moderate |
| MEK | 2.2 < S < 7.3 | Moderate |
| 2-MeTHF | 2.2 < S < 7.3 | Moderate |

### Example 3: Type 8 Preparation and Characterization

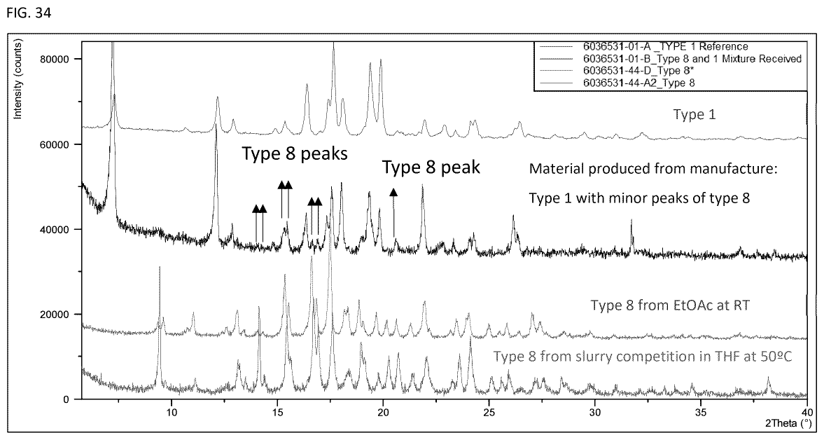

Type 8 is an anhydrous crystalline form and trace amounts of this form was first surprisingly discovered during a drug substance manufacturing campaign-see FIG. 34 for the XRPD acquired from the material produced from the manufacture. The XRPD shows the isolated material is mostly Type 1 with some minor peaks which were attributed to Type 8.

### Competitive Slurry Studies.

Competitive slurry studies with Type 1 and the material produced from manufacture (Type 1 and 8 mixture) were performed at 25 and 50° C. in THF, EtOH and EtOAc. The solid form was checked by XRPD on day 3 and day 7. The results are presented in Table 40.

TABLE 40
Competitive slurry study results

| | | Solid Form | Solid Form |
|---|---|---|---|
| Solvent | Temperature (° C.) | on Day 3 | on Day 7 |
| THF | 25 | Type 8 | Type 8 |
| | 50 | Type 8 | Type 8 |
| EtOH | 25 | Type 8 | Type 8 |
| | 50 | Type 8 | Type 8 |
| EtOAc | 25 | Type 8 | Type 8 |
| | 50 | Type 8 | Type 8 |

The competitive slurry studies indicate that Type 8 is the more thermodynamically stable form at 25 and 50° C.

### Characterization of Type 8

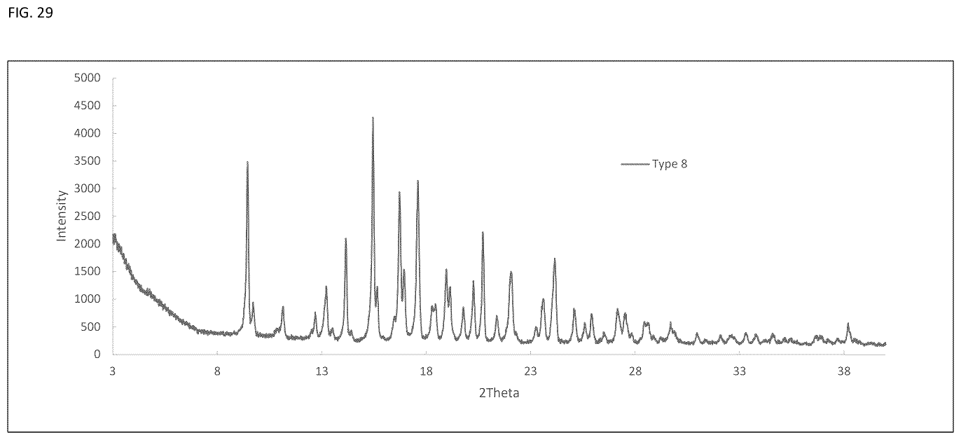

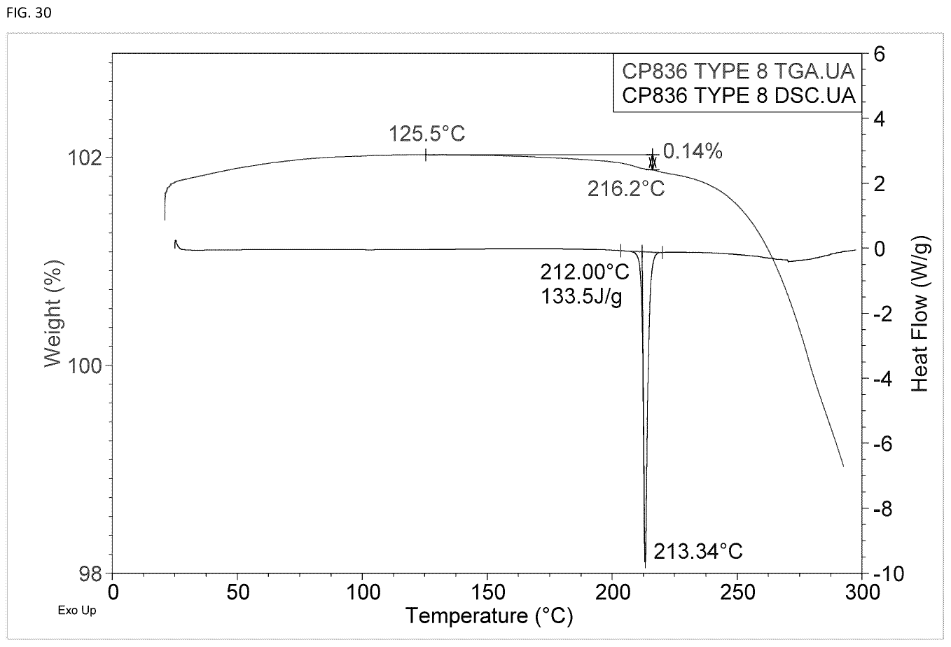

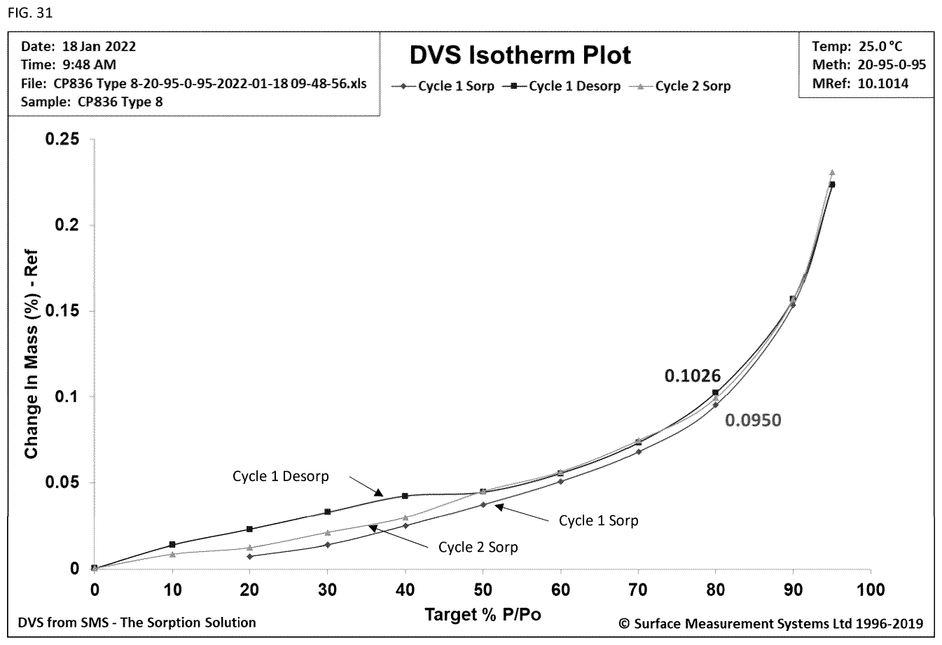

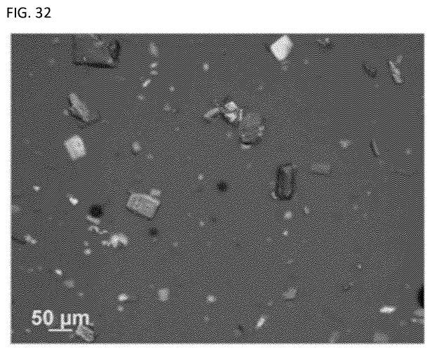

Solid state characterization of Type 8 was performed using XRPD, DVS, DSC, TGA and PLM (see FIG. 29-32). Type 8 was determined to be an anhydrous crystalline form of Compound I(a) (see FIG. 29 for XRPD diffractogram). XRPD peak positions are presented in Table 41. DSC shows a sharp melting endothermic with an onset temperature of 212° C. (see FIG. 30), while TGA shows a weight loss of 0.14% up to 216° C. (see FIG. 30). DVS analysis show water update of 0.1% at 80% RH (see FIG. 31) indicating Type 8 is non-hygroscopic. No change in crystalline form was observed post DVS experiment. PLM shows platelike birefringent particles (FIG. 32).

TABLE 41
XRPD peak positions for Type 8
Peak position in °2θ

3.1
3.5
4.8
5.6
6.1
9.4
9.7
10.7
10.8
11.2
12.7
13.2
13.6
14.2
14.4
15.4
15.7
16.5
16.7
16.9
17.6
18.3
18.5
19.0
19.1
19.8
20.3
20.7
21.4
22.1
23.3
23.6
24.2
25.1
25.6
25.9
26.6
27.1
27.5
27.9
28.4
28.6
29.2
29.7
30.9
31.4
32.1
32.5
32.8
33.4

### Process to Prepare Type 8

Type 8 may be prepared as follows:
1. Charge N-(3-chloro-4-fluorophenyl)-4-((3aS,5S,6aR)-5-hydroxy-5-(3-(2-hydroxy-2-methylpropoxy)-1-methyl-1H-pyrazol-5-yl)-1,3a,4,5,6,6a-hexahydropentalen-2-yl)-1-methyl-1H-imidazole-5-carboxamide (3.9 kg) to reactor.
2. Charge THF (146 kg) to reactor.
3. Charge 10 wt % Pd/C (0.39 kg) in THF (20 kg) to reactor.
4. Expose contents to 1 atm H₂.
5. Stir contents for approximately 25 to 30 hours until no more than 2.0% N-(3-chloro-4-fluorophenyl)-4-((3aS,5S,6aR)-5-hydroxy-5-(3-(2-hydroxy-2-methylpropoxy)-1-methyl-1H-pyrazol-5-yl)-1,3a,4,5,6,6a-hexahydropentalen-2-yl)-1-methyl-1H-imidazole-5-carboxamide remains.
6. Filter contents through celite (wash with THF).
7. Charge activated carbon to filtrate and stir mixture at 50° C. for 16 hours.
8. Filter through celite and filter cartridge (wash with THF).
9. Concentrate to 2-5 volumes over approximately 40 hours at 40 to 50° C. and <1.0 MPa.
10. Charge ethyl acetate (8 volumes).
11. Concentrate to 2-3 volumes over approximately 6-8 hours at 40 to 50° C. and <1.0 MPa.
12. Repeat steps 10 and 11 twice.
13. Dilute mixture with EtOAc (3 volumes).
14. Adjust process temperature to 5-10° C. over approximately 3 hours.
15. Stir contents for 14 to 15 hours.
16. Collect precipitate by filtration (wash with EtOAc).
17. Dry material at 50° C. until Loss on Drying is NMT 4.0%.

### Example 4: Single Crystal Cultivation and Structure Analysis of Compound I

A single crystal of Compound (I) was grown and analyzed by single crystal X-Ray diffraction in order to determine its absolute structure.

The single crystal refers to the regular and periodic arrangement of the particles inside the crystal in three-dimensional space, which is simply a single crystal polyhedron. Single crystal analysis is the most direct and convincing method to identify the absolute structure of compounds. Therefore, single crystal analysis is often used to confirm or even directly determine the absolute structure of drug molecules in drug research.

### Design Experiment of Single Crystal Culture

### Approximate Solubility Test at Room/50° C. Temperature

At room temperature (˜25° C.), the approximate solubility of the starting materials was determined in different solvents. The specific steps are as follows: Charge about 5 mg of Compound I into a clean glass vial, then an appropriate amount of solvent was added at room temperature until the solution has no visible particles (add up to 1.5 mL of solvent), record the amount of solvent at last for the calculation of solubility.

### Slow Cooling Crystallization

At 55° C., charged about 100.0 mg Compound I in a clean glass vial, added 1.2 mL MeOH and a clear solution was obtained. The system was cooled down to 45° C., a small amount of seed was added, and a suspension was obtained. The suspension was filtered through a 0.22 μm filter, and the filtrate was put in a clean glass vial and sealed. The sample was let stand and cooled down to room temperature slowly.

### Methodology and Instruments Used

SCXRD Device Type: Bruker APEX-II CCD
   Diffraction radiation type: Mo K\a
   Diffraction ambient temperature: 298 K

### Experimental Results

### Approximate Solubility

The experimental results of approximately solubility at room temperature are shown in Table 42:

TABLE 42
Approximate solubility

| | Solubility (mg/mL) | |
|---|---|---|
| Solvent | 20-25° C. | 50° C. |
| MeOH | 32.2 < S < 33.3 | 74.1 < S < 76.9 |
| EtOH | 8 < S < 8.3 | 20 < S < 20.4 |
| IPA | S < 3.3 | 5 < S < 5.1 |
| EA | S < 3.3 | S < 3.3 |
| IPAc | S < 3.3 | S < 3.3 |
| Acetone | S < 3.3 | 3.8 < S < 4 |
| THF | 12.5 < S < 12.6 | 18.1 < S < 18.5 |
| MTBE | S < 3.3 | S < 3.3 |
| ACN | S < 3.3 | S < 3.3 |
| H₂O | S < 3.3 | S < 3.3 |
| Heptane | S < 3.3 | S < 3.3 |
| DCM | S < 3.3 | S < 3.3 |
| Toluene | S < 3.3 | S < 3.3 |
| DMSO | 167 < S < 200 | 250 < S < 333 |
| EtOH/H₂O = 95/5(V/V) | 22.2 < S < 23.2 | 45.4 < S < 47.6 |
| IPA/H₂O = 95/5(V/V) | S < 3.3 | 8 < S < 8.1 |
| Acetone/H₂O = 95/5(V/V) | 5 < S < 5.1 | 6.5 < S < 6.7 |

### Slow Cooling Crystallization

The experimental results of slow cooling crystallization are shown in Table 43.

TABLE 43
Results of slow cooling crystallization

| Solvent | Phenomenon |
|---|---|
| MeOH | Colourless plate crystal |

### Single Crystal Data Analysis

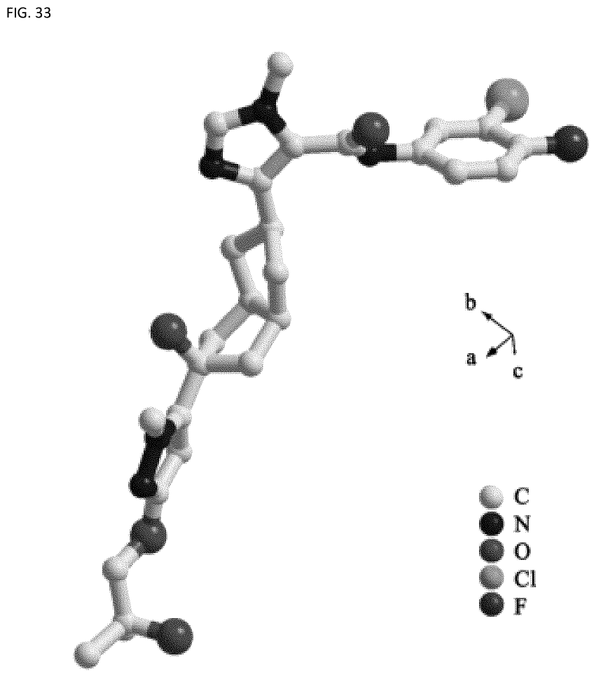

The suitable plate-like single crystal can be grown from the hot saturated MeOH solution through slow cooling crystallization. The single crystal structure of the sample was in monoclinic crystal system, and its space group was P2₁/c. The cell parameters a: 19.5045 Å, b: 10.5123 Å, c: 13.6234 Å; a: 90°, β: 104.549°, y: 90° and V: 2703.7 Å3. Its absolute structure was shown in FIG. 33 and its detailed parameters are shown in Table 44.

TABLE 44
Single crystal parameters

| Chemical formula | C₂₇H₃₃N₅O₄FCl | |
|---|---|---|
| M, g · mol⁻¹ | 546.03 | |
| Crystal system | Monoclinic | |
| Space group | P2_{1/c} | |
| a, Å | 11.629 | (4) |
| b, Å | 12.023 | (7) |
| c, Å | 19.077 | (10) |
| α, deg | 90 | |
| β, deg | 104.549 | (8) |
| γ, deg | 90 | |
| V, Å³ | 2703.7 | (4) |
| Z | 4 | |
| D_{calcd}, g · cm⁻³ | 1.341 | |
| T, K | 298 | (2) |

| | |
|---|---|
| μ, mm⁻¹ | 0.191 |
| GOOF | 0.899 |
| R₁^{a} (I > 2σ(I)) | 0.0706 |
| wR₂^{b} (I > 2σ(I)) | 0.1826 |

The structure of Compound I was determined by single crystal X-Ray diffraction as Compound I(a):

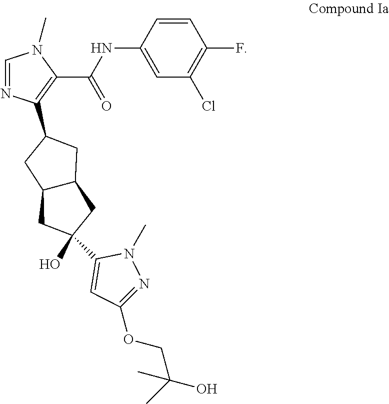 [SMILES: Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

All publications, patents and patent applications cited in this specification are incorporated herein by reference for the teaching to which such citation is used.

Test compounds for the experiments described herein were employed in free or salt form.

The specific responses observed may vary according to and depending on the particular active compound selected or whether there are present carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A crystalline form of Compound I

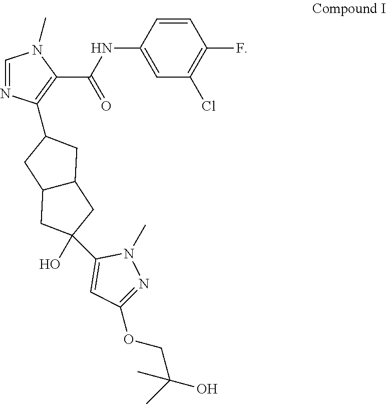 [SMILES: Cn1cnc(C2CC3CC(O)(c4cc(OCC(C)(C)O)nn4C)CC3C2)c1C(=O)Nc1ccc(F)c(Cl)c1]

2. The crystalline form of claim 1, wherein Compound I is Compound I(a):

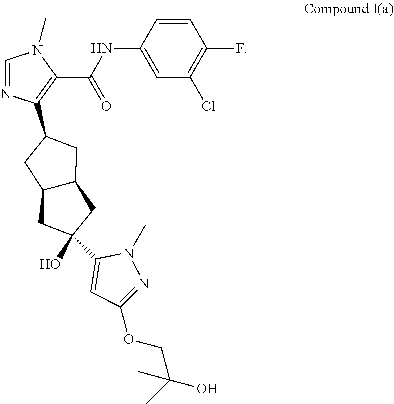 [SMILES: Cn1cnc([C@@H]2C[C@@H]3C[C@@](O)(c4cc(OCC(C)(C)O)nn4C)C[C@@H]3C2)c1C(=O)Nc1ccc(F)c(Cl)c1]

3. The crystalline form of claim 1 or 2, wherein the crystalline form is Type 8.

4. The crystalline form of claim 3, wherein the crystalline form Type 8 is characterized by an XRPD pattern measured using Cu Kα (λ=1.5406 Å) radiation comprising peaks at 2-theta values of 9.4, 15.4 and 16.7 °2θ±0.2° 2θ.

5. The crystalline form of claim 4, wherein the XRPD pattern measured using Cu Kα (λ=1.5406 Å) radiation further comprises at least two, five, ten, fifteen or twenty additional peaks °2θ±0.2° 2θ selected from the group consisting of the following peaks:
Peak Position in °2θ
3.1
3.5
4.8
5.6
6.1
9.7
10.7
10.8
11.2
12.7
13.2
13.6
14.2
14.4
15.7
16.5
16.9
17.6
18.3
18.5
19.0
19.1
19.8
20.3
20.7
21.4
22.1
23.3
23.6
24.2
25.1
25.6
25.9
26.6
27.1
27.5
27.9
28.4
28.6
29.2
29.7
30.9
31.4
32.1
32.5
32.8
33.4

6. The crystalline form of claim 1, wherein Type 8 is characterized by an XRPD pattern measured using Cu Kα (1.5406 Å) radiation substantially the same as shown in FIG. 29

7. The crystalline form of claim 3, wherein the crystalline form is anhydrous.

8. The crystalline form of claim 7 wherein the crystalline form comprises less than 2% by weight of water.

9. The crystalline form of claim 3, wherein the crystalline form Type 8 is characterized by a DSC thermogram comprising an endothermic event with an onset temperature of 212° C.±2° C.

10. The crystalline form of claim 9, wherein the DSC thermogram comprises no thermal events between 5° and 100° C. and an endothermic event with an onset temperature of 212° C.±2° C.

11. (canceled)

12. The crystalline form of claim 3, wherein the crystalline form is substantially pure.

13. The crystalline form of claim 1 or 2, wherein the form is Type 1.

14. (canceled)

15. The crystalline form of claim 13, characterized by an XRPD having at least one of the following 2θ peaks:

| peak | 2Theta (degree) |
|---|---|
| 1 | 7.34 |
| 2 | 12.17 |
| 3 | 16.43 |
| 4 | 17.4 |
| 5 | 17.65 |
| 6 | 18.11 |
| 7 | 19.37 |
| 8 | 19.87 |

16. The crystalline form of claim 15, characterized by at least two, at least three, at least four, at least five, at least six, at least seven of the 2θ peaks, or by the eight 2θ peaks.

17. (canceled)

18. The crystalline form of claim 13, wherein the crystalline form Type 1 is characterized by an XRPD pattern measured using Cu Kα (1.5406 Å) radiation comprising peaks at 2-theta values of 12.2, 19.4 and 19.9° 2θ±0.2° 2θ.

19. The crystalline form of claim 18, wherein the XRPD pattern further comprises at least one or two specific peaks selected from the peaks at 2-theta values of 16.4 and 17.6° 2θ±0.2° 2θ.

20. The crystalline form of claim 18, wherein the XRPD pattern further comprises at least at least two, five, ten, fifteen or twenty additional peaks in ° 2θ±0.2° 2θ selected from the group:
Peak Position in °2θ
4.2
5.0
5.8
6.1
7.3
10.7
11.2
12.9
13.5
14.0
14.9
15.4
16.4
17.0
17.4
17.7
18.1
20.7
20.9
21.4
21.7
21.9
22.9
23.4
24.1
24.3
25.0
26.2
26.5
26.8
27.5
28.1
28.5
29.5
30.2
31.0
32.2
33.1
33.5
34.2
34.4
34.9
35.6
36.2
36.9
37.3
37.8

21. The crystalline form of claim 13, wherein the crystalline form Type 1 is characterized by an XRPD pattern measured using Cu Kα (1.5406 Å) radiation substantially the same as shown in FIG. 24

22. The crystalline form of claim 13, wherein the crystalline form Type 1 is anhydrous.

23. The crystalline form of claim 13, wherein the crystalline form comprises less than 2% by weight of water.

24. The crystalline form of claim 13, wherein the crystalline form Type 1 is characterized by a DSC thermogram comprising an endothermic event with an onset temperature of 210° C.±2° C.

25. The crystalline form of claim 24, wherein the DSC thermogram comprises no thermal events between 5° and 100° C. and an endothermic event with an onset temperature of 210° C.±2° C.

26. (canceled)

27. The crystalline form of claim 13, wherein the Type 1 is at least one of 75%, 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% free of other forms.

28. The crystalline form of claim 13, wherein the Type 1 is free from other forms.

29. The crystalline form of claim 1, wherein the compound is stereochemically pure.

30. A pharmaceutical composition comprising a crystalline form according to claim 1 and a pharmaceutically acceptable carrier, diluent or excipient.

31. A method of treating Hepatitis B (HBV) infection in a subject in need thereof, the method comprising: administering to the subject a therapeutically effective amount of a crystalline form according to claim 1 or a therapeutically effective amount of a pharmaceutical composition comprising the crystalline form and a pharmaceutically acceptable carrier, diluent or excipient.

32. (canceled)

33. (canceled)

34. (canceled)

35. (canceled)
